(19) United States
(12) Patent Application Publication
(10) Pub. No.: US 2024/0352448 A1
(43) Pub. Date: Oct. 24, 2024

(54) Methods and Compositions for Producing Cell-Source Identifiable Collections of Nucleic Acids

(71) Applicant: Takara Bio USA, Inc., San Jose, CA (US)

(72) Inventors: Kazuo Tori, San Jose, CA (US); Magnolia Bostick, San Jose, CA (US); Peng Xu, San Jose, CA (US); Sally X. Zhang, San Jose, CA (US); Szuyuan Eric Pu, San Jose, CA (US); Yue Yun, San Jose, CA (US); Andrew A. Farmer, San Jose, CA (US)

(21) Appl. No.: 18/291,773

(22) PCT Filed: Dec. 22, 2022
(86) PCT No.: PCT/US2022/053883

§ 371 Date: Jan. 24, 2024

Related U.S. Application Data
(60) Provisional application No. 63/293,589, filed on Dec. 23, 2021.

(51) Int. Cl. C12N 15/10

(52) U.S. Cl. CPC C12N 15/1065

(57) ABSTRACT

Provided are methods of preparing source identifiable collections of nucleic acids from a plurality of sources, such as cells or cell nuclei, using a combinatorial indexing methodology. Generally, the methods include providing a first set of cellular source sub-portions, each sub-portion comprising multiple cellular sources of the initial plurality of cellular sources. First identifier tagged nucleic acids are then generated in the multiple cellular sources of each sub-portion of the first set using a template switching mediated reaction employing a template switch oligonucleotide comprising a first identifier, wherein the first identifier of the template switch oligonucleotides employed in the different sub-portions of the first set is the same within a given sub-portion but differs between the different sub-portions. Next, the cellular sources of the sub-portions are pooled to produce a first pool of cellular sources that includes first identifier tagged nucleic acids, and the first pool is then apportioned into a second set of sub-portions each including multiple cellular sources having first identifier tagged nucleic acids. Next, cell-source identifiable nucleic acids from the multiple cellular sources in each sub-portion of the second set that include both a first identifier and a second identifier are produced, wherein the second identifiers of each sub-portion of the second set are the same within a given sub-portion but different between different sub-portions, to prepare a plurality of cell-source identifiable collections of nucleic acids from the initial plurality of cellular sources. The nucleic acids of each cell-source identifiable collection of nucleic acids include a unique combination of first and second identifiers that identifies the cellular source of the nucleic acids. Also provided are kits, compositions and devices, e.g., for use in performing embodiments of the methods as described herein.

### CROSS-REFERENCE TO RELATED APPLICATIONS

Pursuant to 35 U.S.C. § 119(e), this application claims priority to the filing date of the U.S. Provisional Patent Application Ser. No. 63/293,589, filed Dec. 23, 2021, the disclosure of which application is herein incorporated by reference.

### INTRODUCTION

The development of next generation sequencing (NGS) technologies has allowed for the rapid extraction of valuable genomic and transcriptomic information from produced nucleic acid libraries. High throughput NGS technologies, such as the sequencing platforms provided by: Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD™ sequencing system); Roche (e.g., the 454 GS FLX+ and/or GS Junior sequencing systems); and the like, allow for the sequencing of nucleic acid molecules more quickly and cheaply than previously used Sanger sequencing, and as such, these techniques have revolutionized biotechnology and biomedical research. In addition, as these technologies have matured and become more user-friendly, their presence in clinical applications has continued to increase.

These powerful sequencing technologies place a particular emphasis on library preparation. Well-prepared and efficiently produced reverse transcribed complementary DNA (cDNA) libraries can be analyzed using NGS technologies for a diverse range of purposes.

In current NGS workflows, libraries prepared from samples obtained from bulk cell populations or single cells can be sequenced. Sequencing bulk cell populations does not allow for the analysis of genomic and/or transcriptomic changes at single cell resolution, which can mask underlying heterogeneity of different cell types in a bulk population. Where nucleic acid libraries are prepared from individual cells, NGS technologies allow for the analysis of genomic and/or transcriptomic changes at single cell resolution. While single cell sequencing provides for many benefits over bulk cell sequencing, in single cell sequencing one needs to be able to trace a given nucleic acid back to its original source.

While sample barcoding technologies have been developed to address this requirement, there remains upper limits with respect to the number of different cells that can be realistically processed in a given experiment. In some instances, the number of cells that are desirably processed in a single experiment, e.g., where all of the cells are ultimately pooled in a single sequencing ready library composition, exceeds the number that can readily be processed using current protocols. For example, with respect to processing human adaptive immune repertoire samples, single cell analysis allows for pairing chain information (e.g., alpha/beta/gamma/delta pairing for TCR and heavy/light pairing for BCR). However, the number of cells that can be interrogated in a given experiment is limited, e.g., up to 10,000 different cells or may require specialized instrumentation.

### SUMMARY

In such instances, the inventors have realized that what is needed is an approach that allows for the analysis of more than 10,000 cells in parallel. Ideally, what is needed are methods that allow for the analysis of more than 10,000 cells in parallel, and furthermore, where such methods do not require specialized equipment beyond the instrumentation known in the current state of the art. For example, what is needed in the art is an approach that allows for 100,000 or more cells, for example, up to one million single cells, or more than one million cells, to be analyzed in parallel in a single experiment, for example, giving paired chain information for TCR or BCR. In addition, the inventors also recognize that there is a need in the art for methods for performing single cell experiments on multiple samples of single cells wherein the number of single cells in each sample might be low, but the number of independent samples is high, such that the collective number of cells required to be analyzed is large. For example, what is needed in the art is an approach that allows for the analysis of, e.g., 100 samples or more, each sample comprising 1,000 or more cells, such that the total number of cells analyzed is 100,000 or more. As such, there is a continued need for improved single cell sequencing technologies that can provide for processing of large numbers of cells in a given experiment.

Embodiments of the present invention satisfy the above, and other, needs in the art by providing a combinatorial indexing approach to uniquely identify nucleic acids produced from the same cellular source. The combinatorial indexing approach employed by embodiments of the invention described herein represents a substantial improvement over the art by providing for this unique identification without each cell in an assayed population needing to be present individually within a container—separate from other cells being assayed. The provided combinatorial approach allows for the practical analysis of large numbers of single cells, for example, 10,000 or more single cells, 100,000 or more single cells, or one million or more single cells. The provided combinatorial approach applies equally to components thereof, e.g., nuclei, using the same workflow, e.g., where sequencing-ready libraries of nucleic acids from, e.g., 100,000 or more single cells or single nuclei, are pooled and sequenced together, with the resultant sequence information for each read being traceable back to its original cellular source. In addition, the methods for combinatorial analysis provided herein allow for the practical analysis of many independent samples of cells such as, for example, analysis of 100 or more samples, each sample comprising 1,000 or more cells, such that the total number of cells analyzed is 100,000 or more.

Provided are methods of preparing a plurality of cell-source identifiable collections of nucleic acids derived from an initial plurality of cellular sources. Aspects of the methods include providing a first set of cellular source sub-portions, each sub-portion comprising multiple cellular sources of the initial plurality of cellular sources. First identifier tagged nucleic acids are then generated in the multiple cellular sources of each sub-portion of the first set using a template switching mediated reaction employing a template switch oligonucleotide comprising a first identifier (which may also be referred to herein as a first index or first cell barcode), wherein the first identifier of the template switch oligonucleotides employed in the different sub-portions of the first set is the same within a given sub-portion but differs between the different sub-portions. Next, the cellular sources of the sub-portions are pooled to produce a first pool of cellular sources that includes first identifier tagged nucleic acids, and the first pool is then apportioned into a second set of sub-portions each including multiple cellular sources having first identifier tagged nucleic acids. Next, cell-source identifiable nucleic acids are produced from the multiple cellular sources in each sub-portion of the second set that include both a first identifier and a second identifier, wherein the second identifiers of each sub-portion of the second set are the same within a given sub-portion but different between different sub-portions. The methods provide a plurality of cell-source identifiable collections of nucleic acids from the initial plurality of cellular sources. The nucleic acids of each cell-source identifiable collection of nucleic acids include a unique combination of first and second identifiers that identifies the cellular source of the nucleic acids.

In other embodiments, additional rounds of pooling and redistribution to new sub-portions can be employed, as desired, to add additional rounds of indexes (i.e., identifiers) to the collections of nucleic acids. Additional rounds of indexing permits the analysis of greater numbers of individual cells or cellular components such as nuclei in the methodology. Generally, the total number of cells to be examined should be such that the total number of unique combinations and orientations of nucleotide sequence identifiers (e.g., barcodes, indexes, tags, or any type of molecular identifier) is greater than the number of unique cells the researcher wishes to investigate.

Also provided are kits, compositions and devices, e.g., for use in performing embodiments of the methods as described herein.

### BRIEF DESCRIPTION OF THE FIGURES

FIGS. 1A-1D illustrate a workflow according to one embodiment of the invention.

FIG. 2 provides a schematic showing one embodiment of the invention.

FIG. 3 provides a schematic showing one embodiment of the invention.

FIG. 4 provides a schematic showing one embodiment of the invention.

FIG. 5 provides a flowchart showing generally one embodiment of a method of the invention.

FIG. 6 provides a schematic showing the structure of an NGS library product made using an embodiment of the invention, as described in Example 2. More particularly, T cell receptor genes are specifically targeted for analysis. As shown in FIG. 6, Read 1 provides sequence of the targeted TCR gene. Read 2 also provides the sequence of T cell receptor genes together with the first two index sequences, namely: index 2 (IN2; 2^{nd} identifier) from the second indexing step, and index 1 (IN1; first identifier) from the 1st indexing step. Index 3 (3^{rd} identifier) is shown as being provided by the combination of the i7 and i5 indexes added by PCR in the 3rd indexing step.

FIG. 7 provides a schematic showing one embodiment of the invention, specifically, providing a method for preparing a plurality of cell-source identifiable collections of nucleic acids derived from an initial plurality of cellular sources, where the method uses three independent rounds of indexing of the nucleic acids.

FIG. 8A illustrates a two-round barcoding protocol for TCR sequencing (as described in Example 4 below), in accordance with an embodiment of the invention. FIGS. 8B, C, and D: Two-round TCR barcoding. (FIG. 8 Panel B) The experiment design of 8×8 split-pool with 1,000 cells. (FIG. 8 Panel C) Bioanalyzer result from TCRb library. (FIG. 8 Panel D) L-Plot analysis.

FIG. 9 illustrates two-rounds barcoding for TCR sequencing with second round Barcoding split across PCR 1 and PCR2, in accordance with an embodiment of the invention.

FIG. 10A illustrates three rounds barcoding for TCR sequencing (as described in Example 5, below) with third round barcoding split across PCR 1 and PCR2, in accordance with an embodiment of the invention. FIGS. 10B and 10C: Three-round TCR barcoding. (FIG. 10B) Bioanalyzer trace of a TCR library generated as described in Example 5. (FIG. 10C) Table showing the read counts, mapping rate and clonotypes detected in the library.

FIG. 11 illustrates an alternative three-round barcoding strategy for TCR sequencing, in accordance with an embodiment of the invention.

FIG. 12A illustrates two-round barcoding for combined targeted sequencing and 5′ Differential expression (5′DE) (as described in Example 6, below) in accordance with an embodiment of the invention. FIG. 12B illustrates the products and library structure produced using the protocol illustrated in FIG. 12A.

FIG. 12C provides a knee plot used to determine the number of cells detected that pass quality metrics. In this case, cells having >10,000 reads/cell based on data demultiplex using BC1, BC2a, BC2b (i7) and i5 with Cogent AP software. 1310 cells had >10,000 reads and were used for the downstream analysis.

FIG. 12D provides the average mapping statistics of K562 and 3T3 cells, based on mapping to either human hg38 (K562) or mouse mm10 (3T3). The percentage of intergenic, intronic, exonic, multi-mapped, unmapped, & trimmed reads were calculated.

FIG. 12E provides the number of genes detected in either K562 or 3T3 cells as a function of reads per cell.

FIG. 12F provides an L-plot analysis. The sequencing reads of all K562, 3T3 and mixed cells were mapped to both the human (hg38) and mouse (mm10) genomes and plotted based on the number of mapped reads per cell that mapped to each genome.

FIG. 13A illustrates the products and library structure of a TCR library prepared following two rounds of split-pool barcoding, as described in Example 7. FIGS. 13B and C: TCR analysis. (FIG. 13B) Full length cDNA prepared with 10 ng PBMC RNA. (FIG. 13C) TCRa, TCRb and TCRa+b libraries prepared from full length cDNA.

FIG. 14 illustrates a three-round barcoding protocol for combined targeted sequencing and 5′ Differential expression (5′DE) (as described in Example 8, below) in accordance with an embodiment of the invention.

FIG. 15 illustrates libraries and products produced in accordance with various embodiments of the invention. FIG. 15 Panel A depicts 5′DE library generation as described in Example 8. FIG. 15 Panel B depicts TCR library preparation as described in Example 8.

FIG. 16 provides the results of TSO analysis, as described in Example 9.

FIG. 17 provides a knee plot of single cells after demultiplexing, as described in Example 10.

FIG. 18 provides an L-Plot of the human-mouse cell mixture prepared according to an embodiment of the invention as described in Example 10.

FIG. 19 provides an L-Plot of the human-mouse cell mixture processed for combinatorial indexing using a high concentration of PFA and digitonin, as described in Example 11.

### DEFINITIONS

As used herein, the term “hybridization conditions” means conditions in which a primer, or other polynucleotide, specifically hybridizes to a region of a target nucleic acid with which the primer or other polynucleotide shares some complementarity. Whether a primer specifically hybridizes to a target nucleic acid is determined by such factors as the degree of complementarity between the polymer and the target nucleic acid and the temperature at which the hybridization occurs, which may be informed by the melting temperature (T_{M}) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a region of the product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

As used herein, an “oligonucleotide” is a single-stranded multimer of nucleotides from 2 to 500 nucleotides, e.g., 2 to 200 nucleotides. Oligonucleotides may be synthetic or may be made enzymatically, and, in some embodiments, are 10 to 50 nucleotides in length. Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). In some cases, oligonucleotides may contain a mixture of ribonucleotides and deoxyribonucleotides. In some cases, Oligonucleotides may contain modified, i.e., non-natural nucleotides or modifications, including for example, LNA, FANA, 2′-O-Me RNA, 2′-fluoro RNA, or the like, linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the oligonucleotides. Oligonucleotides may be 10 to 20, 21 to 30, 31 to 40, 41 to 50, 51 to 60, 61 to 70, 71 to 80, 80 to 100, 100 to 150 or 150 to 200, up to 500 or more nucleotides in length, for example.

A “domain” when used in reference to nucleic acids refers to a stretch or length of a nucleic acid made up of a plurality of nucleotides, where the stretch or length provides a defined function to the nucleic acid. Examples of domains include barcode domains (such as source barcode domains), primer binding domains, hybridization domains, unique molecular identifier (UMI) domains, Next Generation Sequencing (NGS) adaptor domains, NGS indexing domains, etc. In some instances, the terms “domain” and “region” may be used interchangeably, including e.g., where immune receptor chain domains/regions are described, such as e.g., immune receptor constant domains/regions. While the length of a given domain may vary, in some instances the length ranges from 2 to 100 nt, such as 5 to 50 nt, e.g., 5 to 30 nt. Amplification primer binding domains are domains that are configured to bind via hybridization to an amplification primer.

As used herein, the expression “derived from” describes a composition that results from a process whereby a first component (e.g., a first nucleic acid molecule), or information from that first component, is used to isolate, derive or construct a different second component (e.g., a second nucleic acid molecule that is different in structure, sequence or character from the first nucleic acid molecule from which it was derived). For example, a cDNA molecule is derived from a corresponding mRNA that is found in a cell. Similarly, a DNA library is derived from total RNA that is collected from a cell or population of cells. Also for example, a cDNA library can be derived from mRNA that is collected from a cell or population of cells.

As used herein, the expression “barcode” describes most broadly a short, for example from 6 to 12 nucleotide sequence, which when appended to a larger polynucleotide, serves to tag that larger polynucleotide, thereby providing a means for counting or distinguishing individual nucleic acids in a larger pool of nucleic acids. As used herein, and as recognized by one of skill in the art, a broad array of barcodes and barcoding strategies are widely utilized and described in the prior art, all of which find use in the presently described invention. As used herein, the terms “barcode” or “index” may be used interchangeably with the terms tags, identifier tags, cell barcode, cell barcode sequences, sample barcodes and sample barcode sequences, well barcodes, source barcode sequences, identifiers, molecular identifiers and other similar and equivalent expressions and technologies. The expression “unique molecular identifier” or “UMI” also refers to randomers of varying length, and are also encompassed by the broad meaning of “barcode” as used herein.

### DETAILED DESCRIPTION

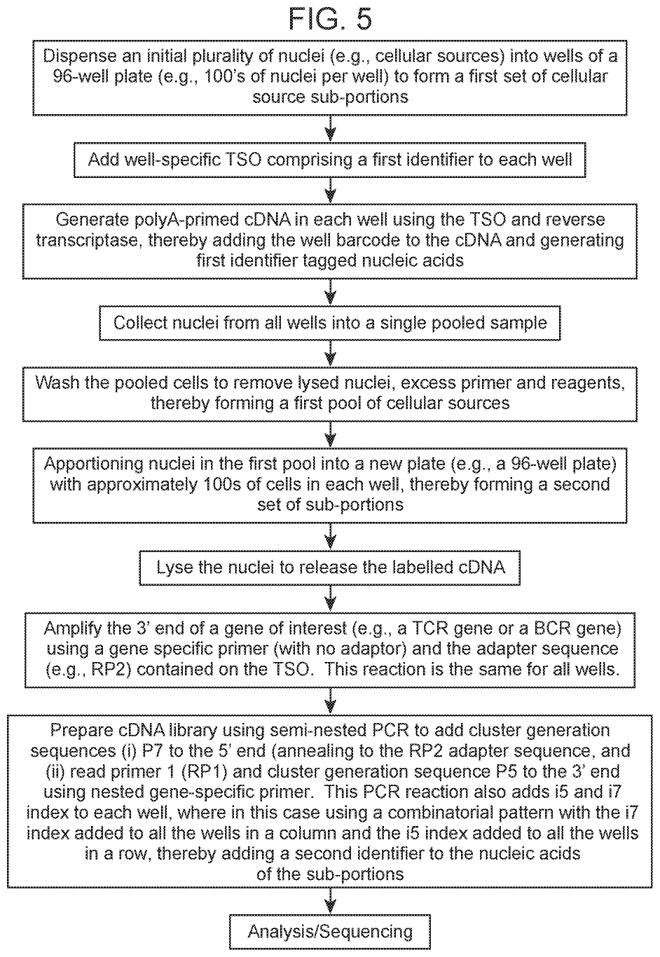

Provided are methods of preparing a plurality of cell-source identifiable collections of nucleic acids from an initial plurality of cellular sources. Aspects of the methods include providing a first set of cellular source sub-portions, each sub-portion comprising multiple cellular sources of the initial plurality of cellular sources. First identifier tagged nucleic acids are then generated in the multiple cellular sources of each sub-portion of the first set using a template switching mediated reaction employing a template switch oligonucleotide comprising a first identifier, wherein the first identifier of the template switch oligonucleotides employed in the different sub-portions of the first set is the same within a given sub-portion but differs between the different sub-portions. Next, the cellular sources of the sub-portions are pooled to produce a first pool of cellular sources that includes first identifier tagged nucleic acids, and the first pool is then apportioned into a second set of sub-portions each including multiple cellular sources having first identifier tagged nucleic acids. Next, cell-source identifiable nucleic acids from the multiple cellular sources in each sub-portion of the second set that include both a first identifier and a second identifier are produced, wherein the second identifiers of each sub-portion of the second set are the same within a given sub-portion but different between different sub-portions, to prepare a plurality of cell-source identifiable collections of nucleic acids from the initial plurality of cellular sources. The nucleic acids of each cell-source identifiable collection of nucleic acids include a unique combination of first and second identifiers that identifies the cellular source of the nucleic acids. One method of the invention is illustrated in the flowchart provided in FIG. 5.

In other embodiments, additional rounds of pooling and redistribution to new sub-portions can be employed, as desired, to add additional rounds of indexes to the collections of nucleic acids. The number of rounds, as well as the total number of individual cellular source sub-portions in each round, are selected in a manner to optimize the method for any particular application of the methodology, that is to say, to suit the objective of the user and reflecting the total number of cells (i.e., the total number of cellular sources) that are optimally to be examined.

Generally, the total number of cells to be examined should be such that the total number of possible unique combinations and orientations of nucleotide sequence identifiers (e.g., barcodes, tags, or any type of molecular identifier) that are added in the first and second addition steps (and optional further additions) is significantly greater than the number of unique cells the researcher wishes to investigate so that there is a high probability that each cell will obtain a unique combination of nucleotide sequence identifiers, and therefor assigned to an individual cellular source. By way of example, if the number of identifiers is 10 times as large as the number of cellular sources, then a doublet rate of approximately 5% will be achieved. By increasing or decreasing the ratio of identifiers to cellular sources, the doublet rate can be increased or decreased as desired.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above and illustrated generally in the figures, the present specification provides methods for preparing a plurality of cell-source identifiable collections of nucleic acids from an initial plurality of cellular sources using combinatorial indexing technology. These methods described herein provide advantages over existing cellular combinatorial indexing protocols, as recognized by one of skill in the art. For example, in the methods described herein, the full length of an mRNA transcript can be combinatorically indexed and assayed without applying long-read sequencing technologies. Furthermore, 5′ end sequences, such as those in immune cell receptors can be specifically targeted for analysis.

### Cellular Sources

As used herein, the expression “cellular source” refers to a cell or any component thereof that contains nucleic acid. When a cell component is used, that component is termed a “nucleic acid providing component.” In some instances, the cellular source can be either a cell or a cell nucleus (where the term nucleus is used in its conventional sense to refer to a membrane-bound organelle that contains a cell's chromosomes).

The initial cellular source from which the cellular source identifiable nucleic acids are produced in accordance with embodiments of the invention may vary and are not particularly limited. Cellular samples from which cellular sources may be obtained may be derived from a variety of sources including but not limited to e.g., a cellular tissue, a biopsy, a blood sample, a cell culture, etc. Additionally, cellular samples may be derived from specific organs, tissues, tumors, neoplasms, or the like. Furthermore, cells from any population can be the source of a cellular source used in the subject methods, such as a population of prokaryotic or eukaryotic single celled organisms including bacteria or yeast. In some instances, the cellular sources utilized in the subject methods may be a mammalian cellular sample, such as a rodent (e.g., mouse or rat) cellular sample, a non-human primate cellular sample, a human cellular sample, or the like. In some instances, a mammalian cellular sample may be mammalian blood sample, including but not limited to e.g., a rodent (e.g., mouse or rat) blood sample, a non-human primate blood sample, a human blood sample, or the like.

When cells from an organism or a cell culture are used, cells of a particular cell type may be preferred, for example, immune system cells, neuronal cells, cardiac cells, tumor cells, or any other cell type. When immune system cells are used, it may be preferable to still further narrow the cell type used in the combinatorial indexing analysis. For example, it may be preferred to limit the analysis to only either B-cells or T-cells. If cells from whole blood are used in the analysis, it may be preferred to use peripheral blood mononuclear cells (PBMCs).

Where the cellular source is a cell nucleus, the nucleus may be obtained from an initial cell using any convenient nucleus isolation protocol. Where the cellular source is a cell, where the cell is not initially isolated, e.g., when the cell is part of a tissue, the cell may be obtained from an initial cell sample, e.g., using any convenient cell isolation protocol.

The number of cells or cell nuclei in the initial plurality of cellular sources is not particularly limited or bounded either with a requirement for a minimum number of cells or nuclei, nor an upper limit for a maximum number of cells or nuclei which might be analyzed, given there is sufficient diversity created by the multiple rounds of indexing that are employed. For example, two, three or more rounds of indexing can be employed in the methods of the invention. While the number of cells or cell nuclei in the initial plurality of cellular sources may vary, in some instances the number of the initial plurality ranges from 2 to 10,000,000, such as 1,000 to 1,000,000 and including from 10,000 to 100,000, where in some instances the number of initial cellular sources in the plurality is 100,000 or greater, such as 1,000,000 or greater. In some embodiments, the initial plurality of cellular sources may comprise any number of distinct, independent, cellular samples, such as from 1 to 100 independent samples or more, such as 100 to 1,000 or more independent samples.

As used herein, by the expression “cell-source identifiable” is meant that the source, e.g., a single cell or a single nucleus, of nucleic acids of a given collection can be determined, such that the nucleic acids of a given population of nucleic acids that are generated from the same cellular source can be traced back to that same or common originating source. In other words, the nucleic acids of a given cell-source identifiable collection are made up of a population of nucleic acids that can be determined to have originated from the same source, e.g., cell or nucleus. Methods of the invention provide for preparation of cell-source identifiable collections of nucleic acids from an initial plurality of cellular sources, where each prepared collection can be traced back to a different cellular source of the plurality. In other words, methods of the invention allow one to prepare a number of nucleic acid collections from a number of initial cellular sources, e.g., cell or nuclei, where each prepared collection can be traced back to, i.e., the nucleic acids can be determined to originate from, its own unique cellular source of the initial plurality. Identifier components of the cell-source identifiable nucleic acids, e.g., first and second identifiers, such as described in greater detail below, allow one to retroactively identify the source, e.g., cell or nucleus, of a given collection of nucleic acids, thereby collectively serving as source barcodes for nucleic acids of the collection. In addition, as will be recognized by one of skill in the art, the cellular sources employed by the present methods are not limited to, including for example, individual cells from a cell culture. The cellular sources employed by the present methods can also be cellular sources from different populations or different samples, for example, but not limited to, different patients, different cell cultures, different treatment groups, different plants from breeding populations or different bacterial species, etc.

### Production of First Set of Sub-Portions

Aspects of methods of embodiments of the invention include providing a first set of cellular source sub-portions, where each sub-portion includes multiple cellular sources of the initial plurality of cellular sources. In this step, the initial plurality of cellular sources is divided into a number of sub-portions, which sub-portions collectively make up the first set and each sub-portion includes a plurality of different cellular sources. In other words, a plurality of sub-portions each made up of a plurality of cellular sources is produced from the initial plurality of cellular sources. Where desired, sub-portions may be present, e.g., reside, in containers or vessels that are isolated from each other by solid barriers, such as wells of a multi-well plate. Alternatively, the sub-portions can be contained in droplets, as known in the art, where, for example, a droplet is distinct from any other droplet in the collection of droplets. While the number of sub-portions making up the first set may vary, in some instances the number ranges from 2 to 25,000 sub-portions, such as 96 to 10,000 sub-portions, for example, 96 to 384 sub-portions. As indicated above, each sub-portion of the first set is made up of, i.e., includes, a plurality of cellular sources. In some embodiments where multiple cellular samples are analyzed, each independent sample is considered to be a sub portion of the initial collection of cellular sources. While the number of cellular sources making up a given sub-portion of the first set may vary, in some instances the number ranges from 1 to 10,000, such as 10 to 1,000 and including from 100 to 1,000.

Sub-portions employed in methods of the invention, e.g., as described above and below, may take a variety of different formats. Sub-portions may take the form of any suitable reaction vessel, including but not limited to e.g., tubes, wells of a multi-well plate, etc. In some instances, sub-portions are wells of a multi-well device (such as e.g., a multi-well plate or a multi-well chip or droplets or the like). In some instances, components necessary for particular reaction steps may be disposed in a reaction vessel prior to the addition of other reagents, e.g., the reaction vessel may be pre-prepared with one or more components of the reaction. For example, a reaction vessel may be pre-prepared with one or more oligonucleotides, including where such oligonucleotides are disposed in the reaction vessel in a hydrated (e.g., in a solution or droplet) or dehydrated (e.g., dried, lyophilized) form. Dehydrated reaction components, e.g., lyophilized oligonucleotides and/or enzymes, may be rehydrated in the reaction vessel prior to use or may be rehydrated during the addition of other reaction components or cellular sources. Reaction vessels that may serve as sub-portions into which the reaction mixtures and components thereof may be added and within which the reactions of the subject methods may take place will vary. Useful reaction vessels include but are not limited to e.g., tubes (e.g., single tubes, multi-tube strips, etc.), wells (e.g., of a multi-well plate (e.g., a 96-well plate, 384 well plate, or a plate with any number of wells such as 2000, 4000, 6000, or 10,000 or more). Multi-well plates may be independent or may be part of a chip and/or device, e.g., as described in greater detail below. For example, a 96-well plate, 384 well plate, or a plate with any number of wells such as 2000, 4000, 6000, or 10000 or more. The multi-well plate can be part of a chip and/or device. The present disclosure is not limited by the number of wells in the multi-well plate. In various embodiments, the total number of wells on the plate is from 100 to 200,000, or from 5,000 to 10,000. In other embodiments the plate comprises smaller chips, each of which includes 5,000 to 20,000 wells. For example, a square chip may include 125 by 125 nanowells, with a diameter of 0.1 mm. The wells (e.g., nanowells) in the multi-well plates may be fabricated in any convenient size, shape or volume. The wells may be 100 μm to 1 mm in length, 100 μm to 1 mm in width, and 100 μm to 5 mm, or more in depth. In some instances, the wells may have a depth of 5 mm or less, including but not limited to e.g., 4 mm or less, 3 mm or less, 2 mm or less, 1 mm or less. In various embodiments, each nanowell has an aspect ratio (ratio of depth to width) of from 1 to 6 or more. In one embodiment, each nanowell has an aspect ratio of 1:6. The transverse sectional area may be circular, elliptical, oval, conical, rectangular, triangular, polyhedral, or in any other shape. The transverse area at any given depth of the well may also vary in size and shape. In certain embodiments, the wells have a volume of from 0.1 nL to 1 μL. The nanowell may have a volume of 1 μL or less, such as 500 nL or less. The volume may be 200 nL or less, such as 100 nL or less. In an embodiment, the volume of the nanowell is 100 nL. Where desired, the nanowell can be fabricated to increase the surface area to volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments. In some embodiments, a multiwell plate, e.g., in the form of an array of addressable nanowells is employed. An example of such a multi-well plate is that which is part of the ICELL8® Single-Cell MSND System (Takara Bio USA). Details of the ICELL8® MSND system are further found in U.S. Pat. Nos. 7,833,709 and 8,252,581, as well as published United States Patent Application Publication Nos. 2015/0362420 and 2016/0245813, the disclosures of which are herein incorporated by reference.

### Generation of First Identifier Tagged Nucleic Acids

Following production of the first set of sub-portions, e.g., as described above, methods of embodiments of the invention include generating first identifier tagged nucleic acids in the multiple cellular sources of each sub-portion of the first set using a template switching mediated reaction. First identifier tagged nucleic acids that are generated or produced in this step are nucleic acids that include a first identifier domain or region. A given first identifier domain may vary in length without limitation, for example, ranging in some instances from 4 to 20 nucleotides (nt), such as 8 to 12 nt, and in some embodiments, will have a sequence that is distinguishable from sequences of other first identifier domains employed in a given method. In some embodiments, the first identifier domain will also be different from second or additional identifier domains employed at subsequent steps of the method. Depending on the protocol employed to generate the first identifier tagged nucleic acids, the first identifier may be present on the first identifier tagged nucleic acids at a location proximal to the end or terminus of the tagged nucleic acids.

In still other embodiments, it is possible that the first barcode (i.e., first identifier domain sequence) and the second or subsequent barcode (i.e., second identifier domain sequence) are the same and can still be used to generate cell-source identifiable collections of nucleic acids. That is to say, the fact that the barcodes are added in different rounds allows them to be distinguished by their order of addition without the need for the barcodes in each round to be different from those in another round of barcoding.

As described above, the first identifier tagged nucleic acids are generated in the multiple cellular sources, by which is meant that the first identifier tagged nucleic acids are produced inside of an intact, though permeabilized, cellular source. As such, where the cellular source is a cell, the first identifier tagged nucleic acids are produced within the cell. Similarly, where the cellular source is a nucleus, the first identifier tagged nucleic acids are produced within the nucleus. To provide for access of reagents employed in this step to the initial template nucleic acids of the cellular source, the cellular source may be permeabilized. Any convenient protocol for permeabilizing the cellular source, e.g., cell or nucleus, may be employed. The term “permeabilize” as used herein means to render permeable the membrane, e.g., cell membrane or nuclear envelope, to reagents employed in the template switch mediated reaction, e.g., template switch oligonucleotides, reverse transcriptases, first strand cDNA primers, etc. The term “permeable” as used herein refers to the ability of enzymes, oligonucleotides (e.g., template switch oligonucleotides or primers), etc., or other material to pass through a lipid bilayer membrane such as a cell membrane or a nuclear envelope, which is the membrane that encloses the nucleus. The term “permeable” can be a relative term to indicate permeability to specific reagents (e.g., of a particular size) with respect to other reagents. In embodiments herein described during the permeabilizing, the cellular source, e.g., cell or nucleus, remains structurally intact. In embodiments described herein, permeabilization can be performed by contacting the cellular source with a chemical agent capable of porating a cell and/or an organelle membrane. In some instances, the chemical agent is a detergent and permeabilization can be performed by contacting the cellular source with a buffer comprising one or more detergents. The term “detergent” as used herein refers to an amphiphilic (partly hydrophilic/polar and partly hydrophobic/non-polar) surfactant or a mixture of amphiphilic surfactants. Detergents can be broadly categorized according to the charge of their polar portion as “anionic” (negative charge; examples including, but not limited to alkylbenzenesulfonates and bile acids, such as deoxycholic acid), “cationic” (positive charge; examples including, but not limited to, quaternary ammonium and pyridinium-based detergents), “nonionic” (no charge; examples including, but not limited to, polyoxyethylene/PEG-based detergents such as Tween and Triton, and glycosidebased detergents such as HEGA and MEGA), and “zwitterionic” (no charge due to equal numbers of positive and negative charges on the detergent molecules; examples including, but not limited to, CHAPS and amidosulfobetaine-type detergents). In some embodiments, suitable detergents for permeabilizing a cell source include, but are not limited to Sodium Dodedcyl Sulfate (SDS), digitonin, leucoperm, saponin, and tween 20. In some embodiments, suitable detergents for permeabilizing a nucleus include, but are not limited to nonionic detergents, Triton X-100, Nonidet-P40, Ionic detergents, Sodium Dodedcyl Sulfate (SDS), deoxycholate, sarkosyl and additional detergents identifiable by a skilled person. Suitable concentration of detergents for permeabilizing cells and organelles such as nuclei comprise various concentrations depending on the detergent (see e.g., sodium dodecyl sulfate at a final concentration up to 1%). Additional information on common detergents including their critical micelle concentration values (CMCs) and other properties can be found in “Detergents: Handbook & Selection Guide to Detergents & Detergent Removal” available from G-Biosciences (2018); Neugebauer, Detergents: An overview, in Methods in Enzymology, M. P. Deutscher, Editor (1990) Academic Press. p. 239-253; and Schramm et al., Surfactants and their applications. Annual Reports Section “C” (Physical Chemistry), 2003. 99(0): p. 3-48; where such information readily allows the skilled user to fine-turn their detergent's concentrations to make sure it doesn't exceed the CMC and cause full lysis of the cell/organelle. As will be understood by a person skilled in the art, in embodiments herein described permeabilization allows for enzymes and other reagents to passively cross the cell membrane and perform enzymatic reactions in-cell or in-organelle, while cellular materials, such as nucleic acids (e.g., mRNAs and genomic DNA), remain trapped within the cell or nucleus and do not diffuse out.

In embodiments of methods described herein, following permeabilization, the cell or organelle, e.g., nucleus, is contacted with reagents capable of producing in-cell or in-organelle, e.g., in-nucleus, first identifier tagged nucleic acids inside of the cellular source. As summarized above, the first identifier tagged nucleic acids are generated in the cellular source using a template switch mediated reaction. By “template switch mediated” reaction is meant a nucleic acid synthesis reaction in which a polymerase switches from a template nucleic acid to a template switch oligonucleotide. As such, in methods of the invention, the first identifier tagged nucleic acids are produced in the cellular source using a template switch oligonucleotide and suitable polymerase. A template switch oligonucleotide is an oligonucleotide utilized in a template switching reaction, e.g., reverse transcription of an RNA template or reverse transcription of a DNA template. As such, production of an identifier tagged nucleic acid may utilize template switching and the ability of certain nucleic acid polymerases to “template switch” i.e., use a first nucleic acid strand as a template for polymerization, and then switch to a second template nucleic acid strand (which may be referred to as a “template switch nucleic acid” or an “acceptor template”) while continuing the polymerization reaction. The result is the synthesis of a hybrid nucleic acid strand with a 5′ region complementary to the first template nucleic acid strand and a 3′ region complementary to the template switch nucleic acid. Methods and reagents related to template switching are also described in U.S. Pat. Nos. 9,410,173 and 10,941,397; the disclosures of which are incorporated herein by reference in their entirety.

The methods of the present disclosure make use of a template switch oligonucleotide in production of a first identifier tagged nucleic acid by template switching. As such, embodiments of the method include contacting a permeabilized cellular source with reagents sufficient to produce first identifier tagged nucleic acids via a template switching reaction in the cellular source, where such reagents may include a template switch oligonucleotide, a template switching polymerase, a first strand primer, etc., such as described in greater detail below.

By “template switch oligonucleotide” is meant an oligonucleotide template to which a polymerase switches from an initial template (e.g., template nucleic acid (e.g., an RNA template or a DNA template)) during a nucleic acid polymerization reaction. In this regard, the template may be referred to as a “donor template” and the template switch oligonucleotide may be referred to as an “acceptor template.” The template switch oligonucleotide may include one or more nucleotides (or analogs thereof) that are modified or otherwise non-naturally occurring. For example, the template switch oligonucleotide may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the template switch oligonucleotide.

In certain aspects, the template switch oligonucleotide includes a 3′ hybridization domain located at its 3′ end. The 3′ hybridization domain may vary in length, and in some instances ranges from 2 to 10 nucleotides in length, such as 3 to 7 nts in length. The 3′ hybridization domain of a template switch oligonucleotide may include a sequence complementary to a non-templated sequence, e.g., a deoxycytidine stretch added to the 3′ end of newly synthesized reverse transcribed first strand cDNA. Non-templated sequences, described in more detail below, generally refer to those sequences that do not correspond to and are not templated by a template, e.g., a RNA template or a DNA template. Where present in the 3′ hybridization domain of a template switch oligonucleotide, non-templated sequences may encompass the entire 3′ hybridization domain or a portion thereof. In some instances, a non-templated sequence may include or consist of a hetero-polynucleotide, where such a hetero-polynucleotide may vary in length from 2 to 10 nts in length, such as 3 to 7 nts in length, including 3 nts. In some instances, a non-templated sequence may include or consist of a homo-polynucleotide, where such a homo-polynucleotide may vary in length from 2 to 10 nts in length, such as 3 to 7 nts in length, including 3 nts. According to some embodiments, the polymerase (e.g., a reverse transcriptase such as MMLV RT) combined into the reaction mixture has terminal transferase activity such that a homo-nucleotide stretch (e.g., a homo-trinucleotide, such as C-C-C) may be added to the 3′ end of a nascent strand, and the 3′ hybridization domain of the template switch oligonucleotide includes a homonucleotide stretch (e.g., a homo-trinucleotide, such as G-G-G) complementary to that of the 3′ end of the nascent strand. In other aspects, when the polymerase having terminal transferase activity adds a nucleotide stretch to the 3′ end of the nascent strand (e.g., a trinucleotide stretch), the 3′ hybridization domain of the template switch oligonucleotide includes a hetero-trinucleotide stretch comprising cytosine and guanine (e.g., an r(C/G)₃ oligonucleotide), which hetero-trinucleotide stretch of the template switch oligonucleotide is complementary to the 3′ end of the nascent strand. Examples of 3′ hybridization domains and template switch oligonucleotides are further described in U.S. Pat. No. 5,962,272, the disclosure of which is herein incorporated by reference.

In addition to the 3′ hybridization domain (located at the 3′ end of the template switch oligonucleotide), the template switch oligonucleotide further includes a first identifier domain, e.g., as described above. In some embodiments, the first identifier domain is positioned on the template switch oligonucleotide at location 3′ of the 5′ end, and therefore 5′ of the 3′ hybridization domain. For all of the given cellular sources of a sub-portion of the first set, the same template switch oligonucleotide having the same first identifier domain is employed in the template switching mediated reaction. As such, the first identifier tagged nucleic acids produced in each cellular source of each sub-portion have the same or common first identifier domain. However, the template switch oligonucleotides employed with different sub-portions of a first set differ from each other at least with respect to the sequence of their first identifier domains, such that the first identifier domain of tagged nucleic acids of a first sub-portion can be distinguished from the first identifier domain of tagged nucleic acids of any other sub-portion of the first set. As such, the first identifier of the template switch oligonucleotides employed in the different sub-portions of the first set is the same within a given sub-portion but differs between the different sub-portions. Within a given workflow, the number of different template switch oligonucleotides that differ from each other with respect to the sequence of their first identifiers may vary and may be commensurate with the number of first sub-portions of a given first set, ranging in some instances from 2 to 25,000 different template switch oligonucleotides, such as 2 to 25,000 different template switch oligonucleotides, including 96 to 10,000 different template switch oligonucleotides. In general, there should be as many distinct template switch oligonucleotides as there are first sub-portions.

According to some embodiments, the template switch oligonucleotide includes a modification that prevents the polymerase from switching from the template switch oligonucleotide to a different template nucleic acid after synthesizing the compliment of the 5′ end of the template switch oligonucleotide (e.g., a 5′ adapter sequence of the template switch oligonucleotide). Useful modifications include, but are not limited to, an abasic lesion (e.g., a tetrahydrofuran derivative), a nucleotide adduct, an iso-nucleotide base (e.g., isocytosine, isoguanine, and/or the like), and any combination thereof.

In some instances, template switch oligonucleotides may include a unique molecular identifier. The terms “unique molecular identifiers” and “UMIs” refer to randomers of varying length, e.g., ranging in length in some instances from 6 to 12 nts, that can be used for counting of individual molecules of a given molecular species. In some instances, counting is facilitated by attaching UMIs from a diverse pool of UMIs to individual molecules of a target of interest such that each individual molecule receives a unique UMI. In such instances, by counting individual transcript molecules, PCR bias generated during NGS library prep can be corrected for, and a more quantitative understanding of the sample population can be achieved. UMIs may, in some instances, be used in conjunction with other barcode sequences such as a source barcode sequence (e.g., cell barcode sequences, sample barcode sequences, well barcode sequences and the like). When present on template switch oligonucleotides, within a given sub-portion a population of different template switch oligonucleotides may be employed, where this population of template switch oligonucleotides may have the same or common first identifier domain but differ from each other with respect to the sequence of their UMI domains. In such instance, the number of different template switch oligonucleotides that differ from each other with respect to their UMI domains but share a common first identifier domain that is provided to a given sub-portion may vary.

In some instances, a template switch oligonucleotide may include an adapter domain (e.g., a defined nucleotide sequence 5′ of the 3′ hybridization domain and first identifier domain of the template switch oligonucleotide) The adapter domain may serve various purposes in downstream applications. In some instances, the adapter domain may serve as a primer binding site for further amplification or, e.g., nested amplification or suppression amplification, e.g., for introducing additional domains, such as may be employed in NGS applications (such as described below in greater detail). In some instances, the template switch oligonucleotide includes a sequencing platform adapter construct. By “sequencing platform adapter construct” is meant a nucleic acid construct that includes at least a portion of a nucleic acid domain (e.g., a sequencing platform adapter nucleic acid sequence) utilized by a sequencing platform of interest, such as a sequencing platform provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Thermo Fisher Scientific (e.g., a SOLiD sequencing system); or any other sequencing platform of interest. In certain aspects, the sequencing platform adapter construct includes a nucleic acid domain selected from: a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); a molecular identification domain (e.g., a molecular index tag, such as a randomized tag of 4, 6, or other number of nucleotides) for uniquely marking molecules of interest to determine expression levels based on the number of instances a unique tag is sequenced; or any combination of such domains. In certain aspects, a barcode domain (e.g., sample index tag) and a molecular identification domain (e.g., a molecular index tag) may be included in the same nucleic acid. The sequencing platform adapter constructs may include nucleic acid domains (e.g., “sequencing adapters”) of any length and sequence suitable for the sequencing platform of interest. In certain aspects, the nucleic acid domains are from 4 to 200 nucleotides in length. For example, the nucleic acid domains may be from 4 to 100 nucleotides in length, such as from 6 to 75, from 8 to 50, or from 10 to 40 nucleotides in length. According to certain embodiments, the sequencing platform adapter construct includes a nucleic acid domain that is from 2 to 8 nucleotides in length, such as from 9 to 15, from 16-22, from 23-29, or from 30-36 nucleotides in length. Examples of such adapter domains, including sequencing platform adapter constructs, that may be present include, but are not limited to, those described in U.S. Pat. Nos. 9,719,136; 10,415,087; 10,781,443; 10,941,397; 10,954,510; and 11,124,828; the disclosures of which are herein incorporated by reference.

In certain aspects, a sequencing platform adapter construct includes a nucleic acid domain that is a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind). The sequencing platform adapter constructs may include nucleic acid domains (e.g., “sequencing adapters”) of any length and sequence suitable for the sequencing platform of interest. In certain aspects, the nucleic acid domains are from 4 to 200 nts in length. For example, the nucleic acid domains may be from 4 to 100 nts in length, such as from 6 to 75, from 8 to 50, or from 10 to 40 nts in length. According to certain embodiments, the sequencing platform adapter construct includes a nucleic acid domain that is from 2 to 8 nts in length, such as from 9 to 15, from 16-22, from 23-29, or from 30-36 nts in length.

The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′)(SEQ ID NO:01), P7 (5′-CAAGCAGAAGACGGCATACGAGAT-3′)(SEQ ID NO:02), Read 1 primer (5′-ACACTCTTTCCCTACACGACGCTCTTCCGATCT-3′)(SEQ ID NO:03) and Read 2 primer (5′-GTGACTGGAGTTCAGACGTGTGCTCTTCCGATCT-3′)(SEQ ID NO:04) domains employed on the Illumina®-based sequencing platforms. Other example nucleic acid domains include the A adapter (5′-CCATCTCATCCCTGCGTGTCTCCGACTCAG-3′)(SEQ ID NO:05) and P1 adapter (5′-CCTCTCTATGGGCAGTCGGTGAT-3′)(SEQ ID NO:06) domains employed on the Ion Torrent™-based sequencing platforms.

The nucleotide sequences of adapter constructs useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct may be designed to include all or a portion of one or more nucleic acid domains in a configuration that enables sequencing the nucleic acid insert (corresponding to the template nucleic acid) on the platform of interest. Sequencing platform adaptor constructs that may be included in a non-templated sequence as well as other nucleic acid reagents described herein, are further described in U.S. patent application Ser. No. 14/478,978 published as US 2015-0111789 A1 and issued as U.S. Pat. No. 10,941,397, the disclosure of which is herein incorporated by reference.

In addition to the template switch oligonucleotide, reagents provided to be, e.g., contacted with, the permeabilized cellular sources of the sub-portions of the first set may include a first strand primer (i.e., a single product nucleic acid primer, e.g., for priming synthesis from a template nucleic acid, e.g., from an RNA template or a DNA template. The first strand primer (single product nucleic acid primer) includes a template binding domain. For example, the nucleic acid may include a first (e.g., 3′) domain that is configured to hybridize to a template nucleic acid, e.g., mRNA, a ssDNA, etc., and may or may not include one or more additional domains which may be viewed as a second (e.g., 5′) domain that does not hybridize to the template nucleic acid, e.g., a non-template sequence domain as described in more detail below. The sequence of the template binding domain may be independently defined or arbitrary. In certain aspects, the template binding domain has a defined sequence, e.g., poly dT or gene specific sequence. In other aspects, the template binding domain has an arbitrary sequence (e.g., a random sequence, such as a random hexamer sequence). In yet other instances, the template binding domain may be quasi-random, e.g., as described in U.S. Pat. No. 8,206,913, the disclosure of which is herein incorporated by reference. While the length of the template binding domain may vary, in some instances the length of this domain ranges from 5 to 50 nts, such as 6 to 25 nts, e.g., 6 to 20 nts. The first strand primer may include one or more nucleotides (or analogs thereof) that are modified or otherwise non-naturally occurring. For example, the single product nucleic acid primer may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the single product nucleic acid primer. In some instances, a first strand primer (i.e., single product nucleic acid primer) may include an adapter domain (e.g., a defined nucleotide sequence 5′ of the 3′ template binding domain of the single product nucleic acid primer), the adapter domain may serve various purposes in downstream applications. In some instances, the adapter domain may serve as a primer binding site for further amplification, as described herein.

In addition to a template switch oligonucleotide and first strand primer, reagents provided to be, e.g., contacted with, the permeabilized cellular sources of the sub-portions of the first set may include a polymerase that is capable of template switching, where the polymerase uses a first nucleic acid strand as a template for polymerization, and then switches to the 3′ end of a second template nucleic acid strand, i.e., to continue the same polymerization reaction. In some instances, the polymerase capable of template switching is a reverse transcriptase. Reverse transcriptases capable of template-switching that find use in practicing the subject methods include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants derivatives, or functional fragments thereof, e.g., RNase H minus or RNase H reduced enzymes. For example, the reverse transcriptase may be a Moloney Murine Leukemia Virus reverse transcriptase (MMLV RT) or a Bombyx mori reverse transcriptase (e.g., Bombyx mori R2 non-LTR element reverse transcriptase). Polymerases capable of template switching that find use in practicing the subject methods are commercially available and include SMARTScribe™ reverse transcriptase and PrimeScript™ reverse transcriptase available from Takara Bio USA (San Jose, CA). In addition to a template switching capability, the polymerase may include other useful functionalities. For example, the polymerase may have terminal transferase activity, where the polymerase is capable of catalyzing the addition of deoxyribonucleotides to the 3′ hydroxyl terminus of an RNA or DNA molecule. In certain aspects, when the polymerase reaches the 5′ end of the template, the polymerase is capable of incorporating one or more additional nucleotides at the 3′ end of the nascent strand not encoded by the template. For example, when the polymerase has terminal transferase activity, the polymerase may be capable of incorporating 1, 2, 3, 4, 5, 6, 7, 8, 9, 10 or more additional nucleotides at the 3′ end of the nascent strand. All of the nucleotides may be the same (e.g., creating a homonucleotide stretch at the 3′ end of the nascent strand) or one or more of the nucleotides may be different from the other(s) (e.g., creating a heteronucleotide stretch at the 3′ end of the nascent strand). In certain aspects, the terminal transferase activity of the polymerase results in the addition of a homonucleotide stretch of 2, 3, 4, 5, 6, 7, 8, 9, 10 or more of the same nucleotides (e.g., all dCTP, all dGTP, all dATP, or all dTTP). For example, according to one embodiment, the polymerase is an MMLV reverse transcriptase (MMLV RT). MMLV RT incorporates additional nucleotides (predominantly dCTP, e.g., three dCTPs) at the 3′ end of the nascent strand. As described in greater detail elsewhere herein, these additional nucleotides may be useful for enabling hybridization between a 3′ hybridization domain of a template switch oligonucleotide and the 3′ end of the nascent strand, e.g., to facilitate template switching by the polymerase from the template to the template switch oligonucleotide.

Template nucleic acids of cellular sources from which first identifier tagged nucleic acids are produced in the cellular sources may vary. According to certain embodiments, the template nucleic acids are template ribonucleic acids (template RNA). Template RNAs may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (lncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, or any combination of RNA types thereof or subtypes thereof. According to certain embodiments, the template nucleic acids are template deoxyribonucleic acids (template DNA). Template DNAs may be any type of DNA (or sub-type thereof) including, but not limited to, genomic DNA (e.g., prokaryotic genomic DNA (e.g., bacterial genomic DNA, archaea genomic DNA, etc.), eukaryotic genomic DNA (e.g., plant genomic DNA, fungi genomic DNA, animal genomic DNA (e.g., mammalian genomic DNA (e.g., human genomic DNA, rodent genomic DNA (e.g., mouse, rat, etc.), etc.), insect genomic DNA (e.g., drosophila), amphibian genomic DNA (e.g., Xenopus), etc.)), viral genomic DNA, mitochondrial DNA, or any combination of DNA types thereof or subtypes thereof.

Template switching reactions as described above result in the production of a first set of first identifier tagged cellular source sub-portions, where each cellular source sub-portion includes a plurality of cellular sources in which the cellular sources of the plurality house or contain, i.e., have within, first identifier tagged nucleic acids, e.g., reverse transcribed product nucleic acids that include first identifier domains. As reviewed above, the first identifier of the first identifier tagged nucleic acids within the cellular sources of a given sub-portion is the same or common, since it was provided by a template switch oligo having that first identifier. However, among any two sub-portions of the first set, the first identifier of the tagged nucleic acids differs, so that tagged nucleic acids of a first sub-portion can be distinguished from tagged nucleic acids of any other sub-portion of the first set.

Reagents employed in generation of the of first identifier tagged nucleic acids within cellular sources of the sub-portions of the first set may be provided to the cellular sources using any convenient protocol. For example, as indicated above the reagent may be present in the sub-portion vessels (e.g., wells), in dried or liquid form, as desired, prior to introduction of the cellular sources in the vessels. Alternatively, the reagents may be provided to the sub-portion vessels containing the cellular sources, e.g., by manually introducing them into the vessels, by dispensing them into the vessels, e.g., using an automated liquid dispensing system, etc. In some embodiments, a multi-sample nano-dispenser (MSND) system that includes a multiwell plate, e.g., in the form of an array of addressable nanowells, and a sample dispenser is employed. An example of such a MSND system is the ICELL8® Single-Cell MSND System (Takara Bio USA, San Jose, CA). Details of the ICELL8® MSND system are further found in U.S. Pat. Nos. 7,833,709 and 8,252,581, as well as published United States Patent Application Publication Nos. 2015/0362420 and 2016/0245813, the disclosures of which are herein incorporated by reference.

### Pooling/Reapportioning

Following production of a first set of first identifier tagged cellular source sub-portions, the first identifier tagged cellular source sub-portions are combined or pooled to produce a first pool of cellular sources comprising first identifier tagged nucleic acids. The cellular sources of the different sub-portions may be combined or pooled using any convenient protocol. The number of cellular sources in the resultant first pool of cellular sources may vary, and in some instances ranges from 2 to 10,000,000 cells, such as 10,000 to 1,000,000 cells or 10,000 to 100,000 cells.

Following pooling, the resultant first pool of cellular sources is apportioned into a second set of sub-portions each including multiple cellular sources comprising first identifier tagged nucleic acids. In other words, the first pool of cellular sources is divided or separated into multiple sub-portions, which collectively make up the second set of sub-portions, where the different sub-portions include a plurality of, or multiple, cellular sources, where the multiple cellular sources making up the different sub-portions include first identifier tagged nucleic acids, e.g., as described. above. While the number of sub-portions making up the second set may vary, in some instances the number ranges from 2 to 25,000 sub-portions, such as 96 to 10,000 sub-portions, including 96 to 5,184 sub-portions. In some instances, the number of sub-portions in the second set is the same as the number of sub-portions in the first set. As indicated above, each sub-portion of the second set is made up of, i.e., includes, a plurality of cellular sources. While the number of cellular sources making up a given sub-portion of the second set may vary, in some instances the number ranges from 1 to 10,000, such as 100 to 1,000 and including from 100 to 500.

Within a given sub-portion of the second set, multiple cellular sources making up that sub-portion differ from each with respect to the first identifier domain of the first identifier tagged nucleic acid present in the cellular sources. Because of the pooling/reapportioning step, cellular sources from different sub-portions of the first set are combined into the same sub-portion of the second set. Within this same sub-portion, the first identifier tagged nucleic acids in the cellular sources differ from each other with respect to the sequence of the first identifier domains. As such, a given sub-portion of the second set will have a plurality of different first identifier domains, with each different domain present in its own cellular source.

### Production of Cell-Source Identifiable Nucleic Acids

In one embodiment of the invention, following apportionment of the first set of pooled cellular sources into the second set of sub-portions, cell-source identifiable nucleic acids are then produced from the multiple cellular sources in sub-portions of the second set. As reviewed above, cell-source identifiable nucleic acids are nucleic acids whose origin or source can be determined based on identifier sequences present in the nucleic acids, where the identifier sequences include at least first and second identifier sequences. As such, cell-source identifiable nucleic acids include both a first identifier and a second identifier, sequence information obtained from the combination of which allows one to determine the source or origin, i.e., starting cellular source, from which the cell-source identifiable nucleic acid was prepared. As reviewed in greater detail below, the second identifier may be made up of a single domain of contiguous nucleotides, or made up of more than one, e.g., first and second, disparate sub-identifier domains, e.g., depending on the protocol used to prepare the cell-source identifiable nucleic acids. In each sub-portion, the second identifier that is present on the cell-source identifiable nucleic acids is the same. Furthermore, the second identifiers of different sub-portions of the second set are different. As such, the second identifiers of each sub-portion of the second set are the same within a given sub-portion but different between different sub-portions. Accordingly, cell-source identifiable nucleic acids of different sub-portions of the second set can be distinguished from each other by their second identifiers. In some embodiments, the combination of the second identifier associated with the nucleic acids in the second set of sub-portions and the first identifier associated with the nucleic acids in the first set of sub-portions imparts a unique combination of first and second identifiers to each collection of nucleic acids generated from a given cellular source that identifies the cellular source of those nucleic acids. In other embodiments, the combination of the first identifier, second identifier, and any third or additional identifier added in additional rounds of indexing imparts a unique identification to the nucleic acids derived from a particular cellular source from the initial plurality of cellular sources.

By choosing appropriate numbers of initial cellular sources, as well as different first identifiers and second identifiers (and corresponding first sub-portions and second sub-portions), or optionally third or further identifiers from subsequent rounds of indexing, one can readily provide for a number of combinations in which the probability of nucleic acids derived from two different cellular sources having the same first and second identifiers is negligible, i.e., close to zero probability, or less than 5% probability, or less than 2% probability, less than 1% probability, less than 0.1% probability, or less than 0.01% probability.

As indicated above, a second identifier that is incorporated into first identifier tagged nucleic acids in sub-portions of the second set may be made up of a single domain of contiguous nucleotides, or made up of more than one, e.g., first and second, disparate sub-identifier domains, e.g., depending on the protocol used to prepare the cell-source identifiable nucleic acids. As such, in some instances the second identifier may be made up of a single domain of contiguous nucleotides, where the length of such a domain may vary, ranging in some instances from 4 to 20, such as 8 to 12. Second identifiers of this embodiment may be introduced in a number of different ways, e.g., by primers in amplification reactions (which may include one or more amplification rounds, e.g., one or more rounds of PCR), as part of ligated adapters, via tagmentation, etc. In yet other instances, the second identifier is made up of more than one, e.g., first and second, disparate sub-identifier domains. These sub-identifier domains may vary in length, ranging from 4 to 20, such as 8 to 12. Second identifiers of this embodiment may be introduced in a number of different ways, e.g., by primers in amplification reactions (which may include one or more amplification rounds, e.g., one or more rounds of PCR), as part of ligated adapters, via tagmentation, etc. In these embodiments where two or more, e.g., first and second, sub-identifiers are employed to make up a second identifier, the same combination of sub-identifiers will be used for a given sub-population, and different combinations of sub-identifiers will be used for different sub-populations. However, a given first sub-identifier need not be used for only one sub-population. Instead, the same first sub-identifier may be used in different sub-populations as long as it is paired with a different second sub-identifier in each sub-population, such that the combination of first and second sub-identifiers of a given sub-population distinguishes other sub-populations of the second set. In this manner the total collection of sub-identifiers that is employed to produce cell-source identifiable nucleic acids may be less than the total number of sub-populations in the second set, where in some instances the number total number of sub-identifiers is 1 to 30%, such as 1 to 25%, or 3 to 10% of the total number of sub-populations in the second set.

Where desired, prior to production of cell-source identifiable nucleic acids, nucleic acids can be released from the cellular sources in the sub-populations of the second set, e.g., by lysing the cellular sources. Lysis can be achieved by, for example, heating or freeze-thaw of the cellular sources, or by the use of detergents or other chemical methods, or by a combination of these. However, any suitable lysis method can be used. In some instances, a mild lysis procedure can advantageously be used to prevent the release of nuclear chromatin, thereby avoiding genomic contamination of a cDNA library, and to minimize degradation of mRNA. For example, heating the cells at 72° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313).

As indicated above, the cell-source identifiable nucleic acids may be produced in the sub-portions of the second set using any convenient protocol that associates the second identifier (including where the second identifier comprises first and second sub-identifiers) with first identifier tagged nucleic acids of the sub-portions to produce cell-source identifiable nucleic acids that include both first and second identifiers. Examples of such protocols include, but are not limited to, amplification protocols, ligation protocols, tagmentation protocols, second strand synthesis reactions, etc. The reaction mixture components in such protocols are combined under conditions sufficient to produce the desired cell-source identifiable nucleic acid products of the reaction. For example, in some instances, the reaction components of an amplification reaction are combined under conditions sufficient to produce a cell-source identifiable nucleic acid product via one or more rounds of amplification, e.g., one or more PCR rounds. In some instances, the reaction components of a ligation reaction are combined under conditions sufficient to produce a ligated cell-source identifiable product nucleic acid. In yet other instances, reaction components of a tagmentation reaction are combined under conditions sufficient to produce a cell-source identifiable tagmented nucleic acid, which may or may not be used in or subjected to subsequent reaction steps.

Prepared reaction mixtures provide the components necessary to generate conditions sufficient to produce the desired cell-source identifiable product nucleic acids. By “conditions sufficient to produce the desired cell-source identifiable nucleic acids” is meant reaction conditions that permit the relevant nucleic acids and/or other reaction components in the reaction to interact with one another in the desired manner. For example, in some instances, the conditions may be sufficient for nucleic acids of the reaction mixture to hybridize. In some instances, the conditions may be sufficient for an enzyme of the reaction mixture to catalyze a chemical process such as e.g., polymerization, hydrolysis, ligation, tagmentation, etc. Achieving suitable reaction conditions may include selecting reaction mixture components, concentrations thereof, and a reaction temperature to create an environment in which the relevant processes proceed, including e.g., the relevant nucleic acids hybridize with one another in a sequence specific manner, the relevant polymerase polymerizes resulting in elongation of a nucleic acid, etc. In addition to specific nucleic acids (e.g., template nucleic acids, oligonucleotides, primers, etc.) of a reaction the reaction mixture may include buffer components that establish an appropriate pH, salt concentration (e.g., KCl concentration), etc. Conditions sufficient to produce a double stranded nucleic acid complex may include those conditions appropriate for hybridization, also referred to as “hybridization conditions”.

Achieving suitable reaction conditions may include selecting reaction mixture components, concentrations thereof, and a reaction temperature to create an environment in which one or more polymerases are active and/or the relevant nucleic acids in the reaction interact (e.g., hybridize) with one another in the desired manner. In suitable reaction conditions, in addition to reaction components, the reaction mixture may include buffer components that establish an appropriate pH, salt concentration (e.g., KCl concentration), metal cofactor concentration (e.g., Mg²⁺ or Mn²⁺ concentration), and the like, for the extension reaction(s), for example second strand synthesis reactions, and/or template switching to occur. Other components may be included, such as one or more nuclease inhibitors (e.g., an RNase inhibitor and/or a DNase inhibitor), one or more additives for facilitating amplification/replication of GC rich sequences (e.g., GC-Melt™ reagent (Takara Bio USA, Inc. (San Jose, CA)), betaine, DMSO, ethylene glycol, 1,2-propanediol, or combinations thereof), one or more molecular crowding agents (e.g., polyethylene glycol, or the like), one or more enzyme-stabilizing components (e.g., DTT present at a final concentration ranging from 1 to 10 mM (e.g., 5 mM)), and/or any other reaction mixture components useful for facilitating polymerase-mediated extension reactions and/or template-switching.

One or more reaction mixtures may have a pH suitable for amplification (e.g., PCR amplification), ligation, second strand synthesis, or tagmentation. In certain embodiments, the pH of the reaction mixture ranges from 5 to 9, such as from 7 to 9, including from 8 to 9, e.g., 8 to 8.5. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for primer extension reactions may vary according to factors such as the particular polymerase employed, the melting temperatures (Tm) of any primers employed, etc. In some instances, a reverse transcriptase (e.g., an MMLV reverse transcriptase) may be employed and the reaction mixture conditions sufficient for reverse transcriptase-mediated extension of a hybridized primer include bringing the reaction mixture to a temperature ranging from 4° C. to 72° C., such as from 16° C. to 70° C., e.g., 37° C. to 50° C., such as 40° C. to 45° C., including 42° C.

As summarized above, second identifier sequences may be associated with first identifier tagged nucleic acids by a variety of means, e.g., via amplification mediated reactions, ligation mediated reactions, tagmentation mediated reactions, second strand synthesis reactions, iso-thermal amplification reactions, template switching reactions and the like. For example, a second identifier sequence, present on a primer or oligonucleotide, may be incorporated into first identifier tagged nucleic acids during an amplification reaction. In some instances, a second identifier sequence may be directly attached to a first identifier tagged nucleic acid. Methods of directly attaching a non-templated sequence to a nucleic acid will vary and may include but are not limited to e.g., ligation, chemical synthesis/linking, enzymatic nucleotide addition (e.g., by a polymerase with terminal transferase activity), and the like. In yet other instances, second identifier sequences may be associated with first identifier tagged nucleic acids using tagmentation.

Where amplification is employed, association of second identifiers with first identifier tagged nucleic acids in the sub-portions of the second set may make use of one more amplification rounds, e.g., PCR rounds, where primers, e.g., forward and reverse amplification primers, may be used in combination with a suitable amplification polymerase to produce cell-source identifiable nucleic acids that include first and second identifier domains, where the second identifier domains may be made up of a single contiguous domain or two or more sub-domains. In such embodiments, the primers may include primer binding sites configured to hybridize to complementary sites of the first identifier tagged nucleic acids. Primers employed in amplification embodiments will have a template binding domain, which hybridize to a corresponding domain in the first identifier tagged nucleic acids. This template binding domain may be defined, e.g., gene-specific, arbitrary, e.g., random, quasi random, etc., such as described above. A given primer employed in the one or more amplification rounds may include a second identifier component, e.g., entire domain or sub-identifier thereof. For example, where the second identifier is made up of first and second sub-identifiers, each of the first and second primers may include one of the sub-identifiers, e.g., positions 5′ of the primer binding site of the primer. In addition, primers employed in an amplification mediated reaction may include one or more additional domains, as desired. Such additional domains many include, but are not limited to, adaptor domains (e.g., sequencing platform adapter constructs), such as described above.

Amplification mediated reactions for producing cell-source identifiable nucleic acids may also employ a suitable polymerase, e.g., for use in amplifying the primed first identifier tagged nucleic acid, etc. Any convenient amplification polymerase may be employed including but not limited to DNA polymerases including thermostable polymerases. Useful amplification polymerases include e.g., Taq DNA polymerases, Pfu DNA polymerases, Terra™ DNA polymerase, those described in U.S. Pat. No. 6,127,155 (the disclosure of which is incorporated herein by reference in its entirety), derivatives thereof and the like. In some instances, the amplification polymerase may be a hot start polymerase including but not limited to e.g., a hot start Taq DNA polymerase, a hot start Pfu DNA polymerase, and the like. An amplification polymerase may be combined into a reaction mixture such that the final concentration of the amplification polymerase is sufficient to produce a desired amount of product nucleic acid. In certain aspects, the amplification polymerase (e.g., a thermostable DNA polymerase, a hot start DNA polymerase, etc.) is present in the reaction mixture at a final concentration of from 0.1 to 200 units/μL (U/μL), such as from 0.5 to 100 U/μL, such as from 1 to 50 U/μL, including from 5 to 25 U/μL, e.g., 20 U/μL. Nucleic acid reactions, e.g., amplification reactions, of the subject methods may include combining dNTPs into a reaction mixture. In certain aspects, each of the four naturally-occurring dNTPs (dATP, dGTP, dCTP and dTTP) are added to the reaction mixture. For example, dATP, dGTP, dCTP and dTTP may be added to the reaction mixture such that the final concentration of each dNTP is from 0.01 to 100 mM, such as from 0.1 to 10 mM, including 0.5 to 5 mM (e.g., 1 mM). In some instances, one or more types of nucleotide added to the reaction mixture may be a non-naturally occurring nucleotide, e.g., a modified nucleotide having a binding or other moiety (e.g., a fluorescent moiety, a biotin moiety) attached thereto, a nucleotide analog, or any other type of non-naturally occurring nucleotide that finds use in the subject methods or a downstream application of interest.

Reaction mixtures may be subjected to various temperatures to drive various aspects of the reaction including but not limited to e.g., denaturing/melting of nucleic acids, hybridization/annealing of nucleic acids, polymerase-mediated elongation/extension, etc. Temperatures at which the various processes are performed may be referred to according to the process occurring including e.g., melting temperature, annealing temperature, elongation temperature, etc. The optimal temperatures for such processes will vary, e.g., depending on the polymerase used, depending on characteristics of the nucleic acids, etc. Optimal temperatures for particular polymerases, including reverse transcriptases and amplification polymerases, may be readily obtained from reference texts. Optimal temperatures related to nucleic acids, e.g., annealing and melting temperatures may be readily calculated based on known characteristics of the subject nucleic acid including e.g., overall length, hybridization length, percent G/C content, secondary structure prediction, etc.

As mentioned above, amplification mediated reactions for associating second identifiers with first identifier tagged nucleic acids to produce cell-source identifiable nucleic acids may include one or more rounds of amplification, e.g., PCR rounds of application. For example, each first round primer may include a different sub-domain of a second identifier, where amplification of first identifier tagged nucleic acids with such primers produces amplicons that include the first identifier as well as first and second sub-domains that collectively make up the second identifier. Alternatively, a first round of amplification may be employed with primers that amplify a desired fraction of the first identifier tagged nucleic acids, e.g., where amplification is performed with primers that include gene specific template binding domains. Following this first round, a second round of primers that introduces the first and second sub-domains may be performed to produce the cell-source identifiable nucleic acids. The number of amplification arounds employed in a given workflow may vary as desired.

In certain aspects, second identifier sequence is associated with first identifier tagged nucleic acids using a ligation protocol. In these instances, the second identifier sequence may be present on a nucleic acid that is ligated to an end of the first identifier tagged nucleic acid. Any convenient ligase may be employed, e.g., T4 ligase. In some instances, the second identifier may be incorporated into a stem-loop adapter construct that is ligated to the first identifier tagged nucleic acids. Further details regarding such adapters are provided in U.S. Pat. Nos. 7,803,550; 8,071,312; 8,399,199; 8,728,737; 9,598,727; 10,196,686; 10,208,337; and 11,072,823; the disclosures of which are herein incorporated by reference.

In yet other embodiments, the present methods may make use of a tagmentation reaction, and may, e.g., include the use of tagmentation reaction components, to associate second identifier sequences with first identifier tagged nucleic acids. The reaction components and the process of tagmentation employed may vary, as desired. Transposomes, employed in tagmentation, may include a transposase and a transposon nucleic acid that includes a transposon end domain and a second identifier sequence, e.g., second sub-identifier domain. These domains are defined functionally and so may be one in the same sequence or may be different sequences, as required by the researcher. The domains may also overlap, such that part of the second identifier sequence domain may be present in the transposon end domain. Tagmentation processes, transposition-based sequence manipulation, and components that may be employed in a tagmentation or transposition-based reactions are described in, e.g., U.S. Pat. Nos. 10,017,759; 9,790,476; 9,683,230; 9,388,465; 9,238,671; 9,193,999; 8,383,345; 6,294,385; 6,159,736; 5,869,296 and 5,677,170; the disclosures of which are incorporated herein by reference in their entirety. Various tagmentation processes, and/or one or more components thereof, may be adapted for use in the herein described methods. In some instances, a resultant tagmented sample may be subjected to PCR amplification conditions, e.g., using one or more post-tagmentation PCR primers that hybridize to one or more post-tagmentation primer binding sites added during the tagmentation reaction. Post-tagmentation primers may include non-templated sequence(s), such as e.g., sequencing platform adapter construct domains. The non-templated sequence(s) may include any of the nucleic acid domains described elsewhere herein (e.g., a domain that specifically binds to a surface-attached sequencing platform oligonucleotide, a sequencing primer binding domain, a barcode domain, a barcode sequencing primer binding domain, a molecular identification domain, or any combination thereof). Such embodiments find use, e.g., where nucleic acids of the tagmented sample do not include all of the adapter domains useful or necessary for sequencing in a sequencing platform of interest, and the remaining adapter domains are provided by the primers used for the amplification of the nucleic acids of the tagmented sample.

With any protocol employed to produce cell-source identifiable nucleic acids, reagents employed in such protocols may be employed to introduce additional features into the cell-source identifiable nucleic acid products, where such additional features may be features that find use in downstream processing of the cell-source identifiable nucleic acids. For example, where generation of cell-source identifiable nucleic acids is part of a sequencing library generation protocol, additional features incorporated into the cell-source identifiable nuclei acids may include adaptor domains, e.g., as described above, such as sequencing platform adaptor construct domains, such as described above, primer binding domains, e.g., which may be employed in subsequent amplification rounds, e.g., to add sequencing adaptor platform constructs, etc.

### Representative Embodiment

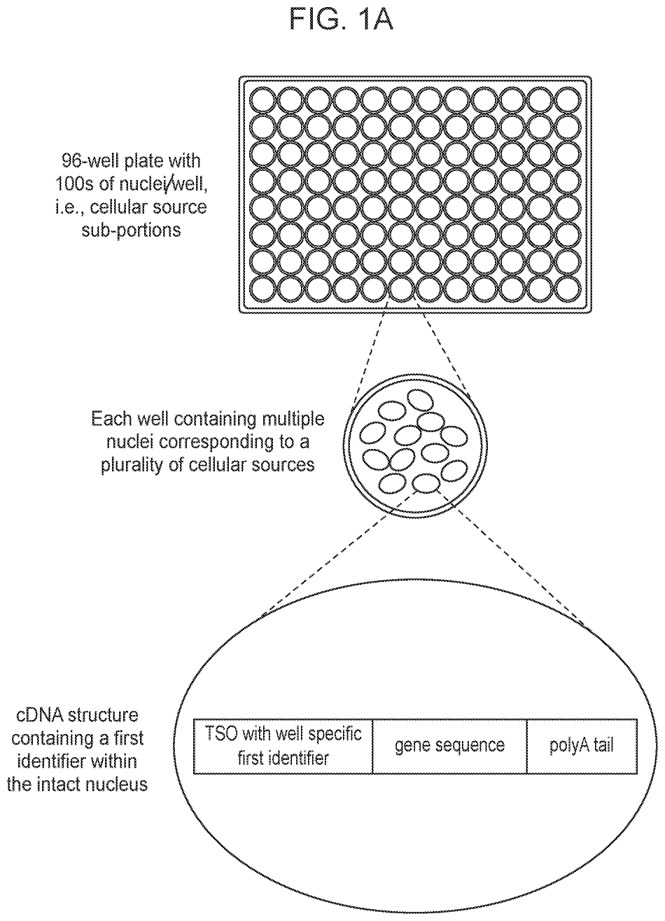

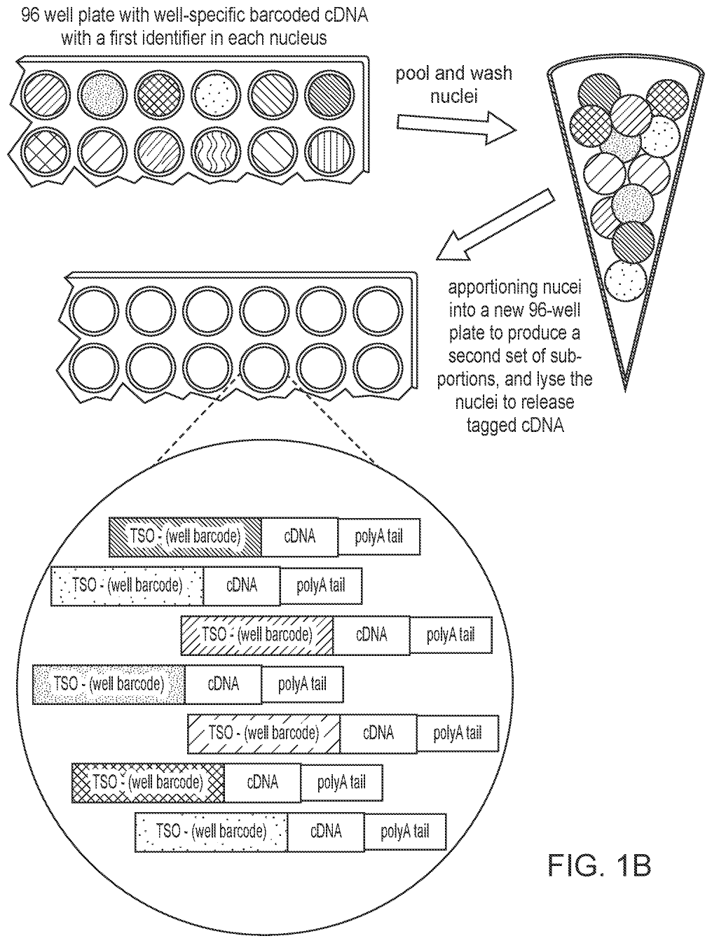

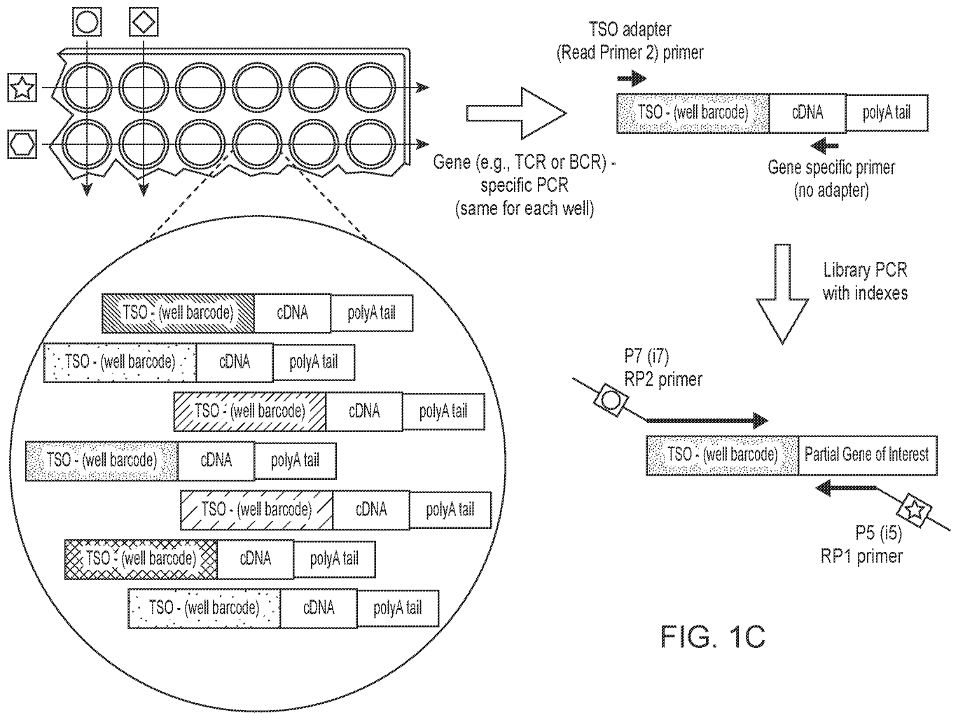

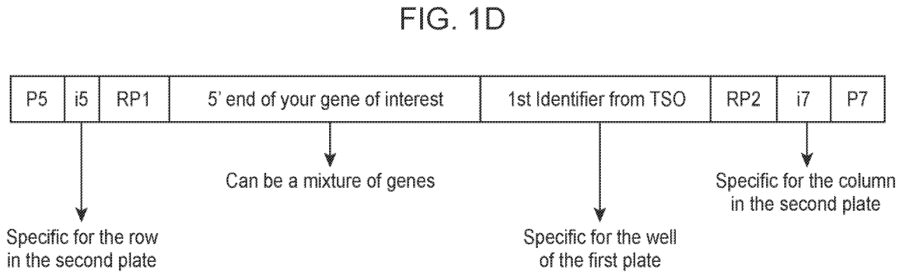

The following section provides a representative embodiment of the invention, in which cell-source identifiable nucleic acids ready for use in a sequencing by synthesis NGS protocol are prepared. This embodiment is shown schematically in FIGS. 1A to 1D which illustrate a workflow for producing cell-source identifiable nucleic acids from an initial plurality of cell nuclei, although whole cells can also be used in place of cell nuclei.

It is important to note that during this protocol, the integrity of the cell source material remains intact. For example, if cells are used in the protocol, then the cells remain intact. If cell nuclei are used in the protocol, the nuclei remain intact.

As illustrated in FIG. 1A, multiple cell nuclei are dispensed into the wells of a 96-well plate. In some embodiments, each well on the 96-well plate receives the cell nuclei, although that is not a requirement of the protocol. Furthermore, the protocol is not limited to the use of 96-well plates, as one of skill recognizes that any suitable vessels, tubes or containers find equal use with the invention, and further for example, multiwell plates having 24 wells or 384 wells or any other multiwell format plate all find use with the invention.

As shown in FIG. 1A, a plurality of nuclei are dispensed into each well of the 96-well plate. For example, a plurality generally on the order of 100 to 1,000 nuclei, for example, 100 nuclei, 200 nuclei or 300 nuclei, are dispensed to each well.

The nuclei are permeabilized either prior to or after distribution to the wells so that combinatorial indexing reagents, for example, the template switching reagents, can enter the nuclei and access the nucleic acids to be analyzed.

Following distribution of the cell nuclei to the wells of the 96-well plate, a well-specific template switch oligonucleotide that includes a unique first identifier is dispensed into each well. Each well receives its own well-specific template switch oligonucleotide (TSO) that differs from the template switch oligonucleotide dispensed into any other well by the sequence of its unique first identifier. Thus, each well receives a different first identifier provided by the well-specific template switch oligonucleotide that is delivered to the well. In addition to the template switching oligonucleotide, template switching reagents, including reverse transcriptase and oligo dT primer, are also delivered to the wells and a reverse transcription reaction is allowed to occur such that first strand cDNA is produced by poly-A priming, where the first strand includes the first identifier sequence of the template switch oligonucleotide at its 3′ end. As a result, each of the nuclei in each well of the multi-well plate contain cDNA molecules corresponding to (i.e., derived from) the mRNA that was in those cell nuclei. As illustrated in the bottom panel of FIG. 1A, the resulting cDNA molecules will each be tagged with a first identifier. That is to say, each cDNA is a first identifier tagged nucleic acid, where the first identifiers of the cDNAs in all of the nuclei in a single well are the same. However, the first identifiers of cDNAs in nuclei in different wells are different.

Continuation of this protocol is shown in FIG. 1B. As shown in FIG. 1B, top left panel, reverse transcription produces a 96 well-plate with cDNAs having well-specific first identifiers, i.e., the cDNA is a first identifier tagged nucleic acid. The first identifier in each nucleus within a single well is the same, but the first identifier differs among the different wells of the 96-well plate, as represented by the different hatch patterns. The nuclei of each well are collected and then pooled in a single tube and washed to remove lysed nuclei and excess primer and RT reagents (FIG. 1B, top right panel). The resultant pooled nuclei are then reapportioned into wells of another 96-well plate, with 100 s of nuclei in each well. The nuclei in each well are then lysed to release the first identifier tagged cDNA present inside the nuclei. As illustrated in FIG. 1B, bottom panel, each well includes collections of cDNAs released from the different lysed nuclei, where the first identifiers of the collections from different wells differ from each other.

This protocol is further illustrated in FIG. 1C. In FIG. 1C, each well of the 96-well plate includes a pool or mixture of first identifier tagged nucleic acids from multiple different original nuclei, as shown, where multiple different first identifiers are present in each well. Well-unique combinations of first and second sub-identifiers that collectively make up a second identifier are then associated with the first identifier tagged nucleic acids in each of the wells. The unique combination of first and second sub-identifiers collectively provide a unique second identifier to each well. As shown in FIG. 1C, a first round of PCR that employs a first gene specific primer and a primer complementary to an adaptor domain, e.g., Read Primer 2 domain, introduced by the TSO, are used to amplify a subset of the first identifier tagged nucleic acids. A second round of amplification is then employed with primers that introduce different sub-domains that collectively make up a second identifier. Unique second identifiers are provided in each well by providing unique combinations of first and second sub-identifiers to each well, where the unique combinations are provided from a more limited set of sub-identifier domains, the number of which is less than the number of wells. In the illustrated approach, a different first sub-identifier is provided to each column of wells on the plate, while a different second sub-identifier is provided to each row of wells on the plate, resulting in each well of the plate having its own unique combination of first and second sub-identifiers. Within each well, the first and second sub-identifiers are associated with the first identifier tagged nucleic acids in the well using an amplification mediated reaction. In the illustrated amplification mediated reaction, a first round of PCR, as discussed above, is performed that amplifies a gene of interest, e.g., a TCR or BCR gene, on the 3′ end (with no adapter) and with the adapter such as RP2 from the TSO, where this round of PCR employs the same primers for all wells. Following this first round of PCR, semi-nested PCR adds cluster generation sequences P7 to the 5′ end (anneal to the RP2 adapter sequence) and Read Primer 1 and cluster generation sequence P5 to the 3′ end (nested gene specific primer). This PCR also adds different i5 and i7 indexes (first and second sub-identifiers) to each well—in this case using a combinatorial pattern with same i7 index added to all the wells in a given column (shown as circle and diamond) and same i5 index added to all the wells in a given row (shown as star and hexagon). Different i7 indices are given to the wells of different columns and different i5 indices are given the wells of different rows. In this manner, unique combinations of different i5 and i7 indices are added to each well, where the indices serve as first and second identifiers. The combination of the i5 and i7 indices collectively associates a unique second identifier with the first identifier tagged nucleic acids of each well. The panel on the bottom right in FIG. 1C shows the reaction that is predicted to occur in the top left well shown in the plate at top left.

FIG. 1D illustrates a cell-source identifiable nucleic acid produced by the protocol illustrated in FIGS. 1A to 1C. As shown in FIG. 1D, the cell-source identifiable nucleic acid is obtained, for example, from the top left well of the 96 well plate shown in FIG. 1C and includes from left to right, a P5 domain, the i5 domain specific for the top left well, the Read Primer 1 domain, the 5′ end of the gene amplified by the gene specific primer, e.g., TCR or BCR gene, amplified in the first round of PCR, the first identifier (i.e., barcode) provided by the template switch oligonucleotide, the Read Primer 2 domain, the i7 domain specific for the top left well, and the P7 domain. All nucleic acids sharing the combination of the same i5 index, TSO first identifier and i7 index can be determined to have been obtained from the same original nucleus. The cell-source identifiable nucleic acid illustrated in FIG. 1D is ready for next generation sequencing (NGS), and following obtainment of the sequence, one can assign a cellular source, i.e., an originating nucleus, to cell-source identifiable nucleic acids of a collection that share the same first identifier and i5,i7 indices based on at least the first and second identifier, e.g., as illustrated by the first identifier, i5 and i7 indices, of the nucleic acids of the collection.

### Iteration

Where desired, a given workflow may further include at least one additional pooling/splitting step to produce nucleic acids that incorporate at least one further identifier. For example, after production of first identifier tagged nucleic acids but prior to lysis of the cellular sources, cellular sources that include identifier tagged nucleic acids present therein may be pooled and apportioned into a set of sub-portions, e.g., as described above. An identifier may be associated with the identifier tagged nucleic acids present in cellular sources of each sub-portion, by any appropriate method, thereby associating another identifier with the identifier tagged nucleic acids. Any number of additional pooling/splitting steps may be employed to provide the desired number of different identifiers in the final cell-source identifiable nucleic acids. Any lysis in a given workflow of such embodiments is reserved for cellular sources in the final set of sub-portions. This final step completes the generation of the source-identifiable nucleic acids which can be identified by their unique combinations of identifiers added in each round of indexing.

### Further Processing

Following production of cell-source identifiable nucleic acids in different sub-portions of the second set, the different sub-portions may be pooled, e.g., to combine the different cell-source identifiable nucleic acids from two or more, including each, sub-portion of the second set into a single composition for further processing. The number of different sub-portions that are combined or pooled in such embodiments may vary, where the number ranges in some instances from 2 to 25,000 or more, such as 96 to 10,000, including 384 to 5,184.

Cell-source identifiable nucleic acids prepared, e.g., as described, may be further processed as desired, e.g., dependent on a particular workflow. For example, cell source identifiable nucleic acids may be prepared for sequencing applications, e.g., next generation sequencing applications. In such instances, the cell-source identifiable collections of nucleic acids that make up the composition may be sequencing ready, in that all domains, e.g., adaptors, such as described above, are already incorporated into the nucleic acids. For example, sequencing platform adapter constructs that may be necessary for use in a given sequencing application may be incorporated into the cell-source identifiable nucleic acids during preparation of the cell-source identifiable nucleic acids, e.g., by inclusion of such on the components employed in the preparation of cell-source identifiable nucleic acids, e.g., template switch oligonucleotides, amplification primers, transposon nucleic acids, etc.

In yet other instances, the cell-source identifiable nucleic acids may be further processed to generate a sequencing ready library, where any convenient approach may be employed in such instances. In such embodiments, one or more of such constructs may be incorporated into the cell-source identifiable nucleic acid acids after the preparation thereof. Where desired, such adapter constructs may be added to a nucleic acid of interest, e.g., a cell-source identifiable nucleic acid, by a variety of means. For example, adapter sequences may be added through the action of a polymerase with terminal transferase activity. Adapter sequences may be incorporated into a nucleic acid during an amplification reaction. In some instances, adapter sequences may be directly attached to a nucleic acid, e.g., to a cell-source identifiable nucleic acid. Methods of directly attaching an adapter sequence to a nucleic acid will vary and may include but are not limited to e.g., ligation, chemical synthesis/linking, enzymatic nucleotide addition (e.g., by a polymerase with terminal transferase activity), tagmentation, and the like.

In some instances, the methods may include attaching sequencing platform adapter constructs, and/or adapters comprising any sequence for any use, to ends of a nucleic acid. For example, in some instances, oligonucleotides and/or primers utilized in the subject methods may not include sequencing platform adapter constructs and thus desired sequencing platform adapter constructs may be attached following the production of a cell-source identifiable nucleic acid of interest. Adapter constructs attached to the ends of a nucleic acid of interest or a derivative thereof may include any sequence elements useful in a downstream sequencing application, including any of the elements described above with respect to the optional sequencing platform adapter constructs of the oligonucleotides and/or primers of the herein described methods. For example, the adapter constructs attached to the ends of nucleic acid of interest or a derivative thereof may include a nucleic acid domain or complement thereof selected from the group consisting of: a domain that specifically binds to a surface-attached sequencing platform oligonucleotide, a sequencing primer binding domain, a barcode domain, a barcode sequencing primer binding domain, a molecular identification domain, and combinations thereof.

Attachment of the sequencing platform adapter constructs may be achieved using any suitable approach. In certain aspects the adapter constructs are attached to the ends of the product nucleic acid or a derivative thereof using an approach that is the same or similar to “seamless” cloning strategies. Seamless strategies eliminate one or more rounds of restriction enzyme analysis and digestion, DNA end-repair, de-phosphorylation, ligation, enzyme inactivation and clean-up, and the corresponding loss of nucleic acid material. Seamless attachment strategies of interest include: the In-Fusion® cloning systems available from Takara Bio USA, Inc. (San Jose, CA), SLIC (sequence and ligase independent cloning) as described in Li & Elledge (2007) Nature Methods 4:251-256; Gibson assembly as described in Gibson et al. (2009) Nature Methods 6:343-345; CPEC (circular polymerase extension cloning) as described in Quan & Tian (2009) PLoS ONE 4(7): e6441; SLiCE (seamless ligation cloning extract) as described in Zhang et al. (2012) Nucleic Acids Research 40(8): e55, and the GeneArt® seamless cloning technology by Life Technologies (Carlsbad, CA).

Any suitable approach may be employed for providing additional nucleic acid sequencing domains to a nucleic acid of interest or derivative thereof having less than all of the useful or necessary sequencing domains for a sequencing platform of interest. For example, a nucleic acid of interest or derivative thereof could be amplified using PCR primers having adapter sequences at their 5′ ends (e.g., 5′ of the region of the primers complementary to the nucleic acid of interest or derivative thereof), such that the amplicons include the adapter sequences in the original nucleic acid as well as the adapter sequences in the primers, in any desired configuration. Other approaches, including those based on seamless cloning strategies, restriction digestion/ligation, tagmentation, or the like may be employed. Methods for adding nucleic acid domains to a Next Generation Sequencing library are known in the art, for example but not limited to, those described in U.S. Pat. No. 11,124,828, the entirety of which is hereby incorporated by reference.

Following prescribed library preparation and/or amplification steps, e.g., as described above, prepared libraries may be considered ready for sequencing. In certain embodiments, the methods provided may further include subjecting a prepared library to an NGS protocol. The protocol may be carried out on any suitable NGS sequencing platform. NGS sequencing platforms of interest include, but are not limited to, a sequencing platform provided by Illumina® (e.g., the HiSeq™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Oxford Nanopore Technologies (ONT); Life Technologies™ (e.g., a SOLiD sequencing system); Roche (e.g., the 454 GS FLX+ and/or GS Junior sequencing systems); or any other sequencing platform of interest. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

Other variations include, e.g., replacing Illumina®-specific sequencing domains in the various primers/oligonucleotides with sequencing domains required by sequencing systems from, e.g., Ion Torrent™ (e.g., the Ion PGM™ and Ion Proton™ sequencing systems); Pacific Biosciences (e.g., the PACBIO RS II sequencing system); Life Technologies™ (e.g., a SOLiD sequencing system); Roche (e.g., the 454 GS FLX+ and GS Junior sequencing systems); or any other sequencing platform of interest.

### Cellular Sources

As described above, the initial cellular source from which the cellular source identifiable nucleic acids are produced in accordance with embodiments of the invention may vary. Cellular samples from which cellular sources may be obtained may be derived from a variety of sources including but not limited to e.g., a cellular tissue, a biopsy, a blood sample, a cell culture, etc. Additionally, cellular samples may be derived from specific organs, tissues, embryos, blastocysts, tumors, neoplasms, or the like. Without limitation, the number of cellular samples that can be analyzed with any embodiment of the invention can vary based on the desire of the researcher. If multiple cellular samples are used, then each independent sample is treated as an initial sub-portion of the initial cellular source. Furthermore, cells from any population can be the source of a cellular source used in the subject methods, such as a population of prokaryotic or eukaryotic single celled organisms including bacteria or yeast. In some instances, the cellular sources utilized in the subject methods may be a mammalian cellular sample, such as a rodent (e.g., mouse or rat) cellular sample, a non-human primate cellular sample, a human cellular sample, or the like. In some instances, a mammalian cellular sample may be mammalian blood sample, including but not limited to e.g., a rodent (e.g., mouse or rat) blood sample, a non-human primate blood sample, a human blood sample, or the like.

In some instances, the cellular source utilized in the subject methods may be an immune cellular source, including but not limited to a source of lymphocytes, e.g., a T cell (e.g., a cytotoxic T cell (e.g., a CD8+ T cell), a helper T cells (e.g., a CD4+ T cell), a regulatory T cells (“Treg”), etc.) a natural killer (NK) cells, a B cells, and the like. Subject immune cells may also include e.g., peripheral blood mononuclear cells, a macrophage, a dendritic cell, a monocyte, etc.

In some instances, the cellular source utilized in the subject methods may be derived from a plant, such as a monocot or a dicot, including but not limited to e.g., research plants (e.g., Arabidopsis) and agricultural plants such as fruits (e.g., apples, apricots, avocados, bananas, blackberries, blueberries, cantaloupe, coconuts, cranberries, dates, figs, melon, grapefruit, grapes, guava, honeydews, kiwifruit, lemons, limes, mangoes, nectarines, olives, oranges, papaya, passion fruit, peaches, pears, pineapples, plantains, plums, pomegranates, prunes, raspberries, strawberries, tangerines, watermelons, etc.), crops (e.g., barley, beans, canola, corn, cotton, flaxseed, hay, oats, peanuts, rice, sorghum, soybeans, sugarbeets, sugarcane, sunflowers, tobacco, wheat, etc.), vegetables (e.g., artichokes, asparagus, beans, beets, bok choy, broccoli, brussels sprouts, cabbage, carrots, cauliflower, celery, collard greens, corn-sweet, cucumbers, eggplant, endive, greens, kale greens, lettuce, parsley, parsnips, peas, peppers, pumpkins, radishes, rhubarb, rutabagas, spinach, squash, sweet potatoes, tomatillos, tomatoes, turnips, water chestnuts, etc.), and the like.

Cellular sources of single cells for use in the herein described methods relating thereto, may be obtained by any convenient method. For example, in some instances, single cells may be obtained through limiting dilution of cellular sample. In some instances, the present methods may include a step of obtaining single cells. A single cell suspension can be obtained using standard methods known in the art including, for example, enzymatically using trypsin or papain to digest proteins connecting cells in tissue samples or releasing adherent cells in culture, or mechanically separating cells in a sample.

In some instances, single cells may be obtained by sorting a cellular sample using a cell sorter instrument. By “cell sorter” as used herein is meant any instrument that allows for the sorting of individual cells into an appropriate vessel for downstream processes, such as those processes of library preparation as described herein. Useful cell sorters include flow cytometers, such as those instruments utilized in fluorescence activated cell sorting (FACS). Flow cytometry is a well-known methodology using multi-parameter data for identifying and distinguishing between different particles (e.g., cell) types, i.e., particles that vary from one another terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately individually to a source of light at a single wavelength (or in some instances two or more distinct sources of light) and measurements of scatter and/or fluorescent parameters, as desired, are separately recorded for each cell. The data recorded for each cell is analyzed in real time or stored in a data storage and analysis means, such as a computer, for later analysis, as desired. Cells sorted using a flow cytometer may be sorted into a common vessel (i.e., a single tube), or may be separately sorted into individual vessels. For example, in some instances, cells may be sorted into individual wells of a multi-well plate, as described below.

### Applications

Methods of preparing cell-source nucleic acids in accordance with the invention, e.g., as described above, may be employed to prepare sequence ready libraries for a variety of different purposes. In certain embodiments, the subject methods may be used to generate an expression library corresponding to mRNAs for downstream sequencing on a sequencing platform of interest (e.g., a sequencing platform provided by Illumina®, Ion Torrent™, Pacific Biosciences, Life Technologies™, Roche, or the like).

A prepared library may be utilized in various downstream analyses and, in some instances the preparation of the library may be specifically reconfigured for a desired type of downstream analysis. For example, in some instances, a prepared library may be subjected to whole transcriptome analysis (WTA) that includes analysis of mRNA as well as non-mRNA RNA species such as non-coding RNA (e.g., Long noncoding RNAs (lncRNAs), non-poly adenylated RNAs, snRNA and snoRNA). Therefore, in some instances, library preparation may be specifically configured to allow for analysis of non-mRNA RNAs within the transcriptome, e.g., by utilizing primers that do not rely on hybridization to the poly(A) tail (e.g., random primers) or by the addition of a tailing reaction, e.g., by adding a poly(A) tail to RNA species that are not naturally polyadenylated prior to production of product double stranded cDNA.

In some instances, preparation of a library, e.g., a library for WTA, may include a step of reducing the amount of ribosomal RNAs within the sample and/or library. This may be done either with the original cell source template nucleic acids before any indexing step of the invention, e.g., using a RiboGone™ product (Takara Bio USA Inc., San Jose, CA) or post generation of indexed fragments (e.g., ZapR technology, for example as described in U.S. Pat. No. 10,150,985), for example, after any indexing step including at the end of all indexing steps prior to sequencing. Any convenient method of reducing and/or removing unwanted ribosomal RNAs may be employed for selective removal, including e.g., using affinity purification, degradation of the contaminating nucleic acid (e.g., using a RiboGone™ product (Takara Bio USA Inc., San Jose, CA) and those methods described in U.S. Pat. Nos. 9,428,794 and 10,150,985, the disclosures of which are incorporated herein by reference in their entirety), combinations thereof, and the like.

In certain embodiments, a prepared library may be utilized in a differential expression analysis, including e.g., where the relative expression (i.e., the up or down regulation) of one or more genes is determined. Differential expression may be qualitatively or quantitatively determined, and such analyses may be transcriptome wide or may be targeted. As such, the number of expressed transcripts evaluated in a subject differential expression analysis will vary. A differential expression analysis as used herein is not limited in regard to the number of expressed transcripts that are analyzed in a subject genome. In some embodiments, a differential expression analysis may evaluate a limited number transcripts such as a panel of marker genes specifically targeted for analysis. Alternatively, the entire transcribed content of the cell may be assessed for differential expression.

Transcript categories to which a targeted expression analysis may be limited will vary and may include but not be limited to e.g., immune gene transcripts, such as cytokines, chemokines or cell surface markers of immune cell subsets, kinases, G-protein coupled receptors, druggable genes, and others. Useful categories and subcategories of immune genes generally include those groups of genes responsible for functioning of the immune system and the successful defense against pathogens, including but not limited to e.g., those genes associated with immune system process (such as the genes identified by gene ontology (GO) accession number GO:0002376 (available online at geneontology(dot)org) including but not limited to e.g., those genes associated with B cell mediated immunity, B cell selection, T cell mediated immunity, T cell selection, activation of immune response, antigen processing and presentation, antigen sampling in mucosal-associated lymphoid tissue, basophil mediated immunity, eosinophil mediated immunity, hemocyte differentiation, hemocyte proliferation, immune effector process, immune response, immune system development, immunological memory process, leukocyte activation, leukocyte homeostasis, leukocyte mediated immunity, leukocyte migration, lymphocyte co-stimulation, lymphocyte mediated immunity, mast cell mediated immunity, myeloid cell homeostasis, myeloid leukocyte mediated immunity, natural killer cell mediated immunity, negative regulation of immune system process, neutrophil mediated immunity, positive regulation of immune system process, production of molecular mediator of immune response, regulation of immune system process, somatic diversification of immune receptors, tolerance induction, and the like. Specific genes of interest include, but are not limited to: cytokines, interleukins, interleukin receptors, CD4, CD8, CD3, PD-1, etc.

In some embodiments the present methods include preparing an immune cell receptor repertoire library from an RNA sample. Aspects of the subject methods include amplifying an immune cell-specific cDNA from a product double stranded cDNA generated from an RNA sample to produce an immune cell receptor repertoire library. By “immune cell receptor repertoire library” is generally meant a nucleic acid library that includes full length or partial sequences of one or more types of immune receptors of a cell or a population of cells. For example, an immune cell receptor repertoire library may be generated for a single cell or for a population of cells derived from a single cellular sample or a single subject or a population of cellular samples, including e.g., a population of samples from two or more subjects. In some instances, a subject library may be generated from individual single cells which, following the addition of an identifying nucleic acid sequence, may be pooled.

As noted above, the members of an immune cell receptor repertoire library may vary in length and may be full length or less than full length. In some instances, the members of the library will preferentially include the 5′ end of an immune cell receptor. Immune cell receptors of interest include but are not limited to e.g., the T-cell receptor (TCR) and the B-cell receptor (BCR).

In some instances, an immune cell receptor repertoire library may include a TCR repertoire library. The TCR complex is a disulfide-linked membrane-anchored heterodimeric protein normally expressed on the surface of T cells and consisting of the highly variable alpha (α) and beta (β) chains expressed as part of a complex with CD3 chain molecules. Many native TCRs exist in heterodimeric αβ or γδ forms. The complete endogenous TCR complex in heterodimeric αβ form includes eight chains, namely an alpha chain (referred to herein as TCRα or TCR alpha), beta chain (referred to herein as TCRβ or TCR beta), delta chain, gamma chain, two epsilon chains and two zeta chains. The alpha and beta TCR chains include variable (V) and constant (C) regions. TCR diversity is generated from genetic recombination (VJ recombination of alpha chains and VDJ recombination of beta chains) resulting in areas of intersection that are important for antigen (i.e., peptide/MHC) recognition.

In some instances, a TCR repertoire library may include TCR-α chain sequences, TCR-β chain sequences, or both TCR-α chain sequences and TCR-β chain sequences. TCR chain sequences of a subject TCR repertoire library may include full length TCR chain sequences (e.g., full length TCR alpha chain sequences, full length TCR beta chain sequences) or partial TCR chain sequences (e.g., partial length TCR alpha chain sequences, partial length TCR beta chain sequences).

Where the subject TCR repertoire library members include partial TCR chain sequences, the partial TCR chain sequences may include the entire or essentially the entire TCR chain variable region (e.g., the TCR alpha chain variable region, the TCR beta chain variable region). In some instances, the resulting library members include the TCR variable region and at least a portion of the TCR constant region. In some instances, the resulting library members include sequence corresponding to the TCR alpha and/or beta chain 5′ mRNA ends. In some instances, the resulting library members include sequence from the TCR alpha or beta chain 5′ end to at least a portion of the corresponding chain constant region.

In certain embodiments, preparation of the immune cell specific library may include TCR specific amplification. Such TCR specific amplification may make use of a TCR specific primer. By “TCR specific primer” is meant a primer that specifically hybridizes to a region of a TCR chain (e.g., a TCR alpha chain, a TCR beta chain) nucleic acid sequence or the complement thereof. In some instances, a TCR specific primer may hybridize to only one type of TCR chain, e.g., only a TCR alpha chain or only a TCR beta chain. In some instances, a TCR specific primer may be configured to hybridize to more than one type of TCR chain, e.g., configured to hybridize to both a TCR alpha chain and a TCR beta chain.

TCR specific primers may be designed to specifically hybridize to a TCR alpha chain constant region or the complement thereof. For example, in some instances, a TCR specific primer may hybridize to a mammalian TCR alpha chain constant region or a complement thereof, including e.g., a human TCR alpha chain constant region, a mouse TCR alpha chain constant region, rhesus monkey, hamster, camel, or the like.

An exemplary human TCR alpha chain constant region has the following amino acid sequence:

(SEQ ID NO: 07)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDINLNFQNLSVIGFRILLLKVAGFNLLMTLRLWSS,

which is encoded by the following nucleic acid sequence:

(SEQ ID NO: 08; T-cell receptor alpha chain
C region, human; GenBank: AY247834.1,
AAO72258.1; UniProtKB: P01848)
CCAAATATCCAGAACCCTGACCCTGCCGTGTACCAGCTGAGAGACTCTAA
ATCCAGTGACAAGTCTGTCTGCCTATTCACCGATTTTGATTCTCAAACAA
ATGTGTCACAAAGTAAGGATTCTGATGTGTATATCACAGACAAAACTGTG
CTAGACATGAGGTCTATGGACTTCAAGAGCAACAGTGCTGTGGCCTGGAG
CAACAAATCTGACTTTGCATGTGCAAACGCCTTCAACAACAGCATTATTC
CAGAAGACACCTTCTTCCCCAGCCCAGAAAGTTCCTGTGATGTCAAGCTG
GTCGAGAAAAGCTTTGAAACAGATACGAACCTAAACTTTCAAAACCTGTC
AGTGATTGGGTTCCGAATCCTCCTCCTGAAAGTGGCCGGGTTTAATCTGC
TCATGACGCTGCGGCTGTGGTCCAGCTGA.

An exemplary mouse TCR alpha chain constant region has the following amino acid sequence:

(SEQ ID NO: 09; UniProtKB: P01849)
PYIQNPEPAVYQLKDPRSQDSTLCLFTDFDSQINVPKTMESGTFITDKTV
LDMKAMDSKSNGAIAWSNQTSFTCQDIFKETNATYPSSDVPCDATLTEKS
FETDMNLNFQNLSVMGLRILLLKVAGFNLLMTLRLWSS
or
(SEQ ID NO: 10; GenBank: AAA53226.1)
PNIQNPEPAVYQLKDPRSQDSTLCLFTDFDSQINVPKIMESGTFITDKTV
LDMKAMDSKSNGAIAWSNQTSFTCQDIFKETNATYPSSDVPCDATLTEKS
FETDMNLNFQNLSVMGLRILLLKVAGFNLLMTLRLWSS

which are encoded by the following nucleic acid sequences, respectively:

(SEQ ID NO: 11)
CCATACATCCAGAACCCAGAACCTGCTGTGTACCAGTTAAAAGATCCTCG
GTCTCAGGACAGCACCCTCTGCCTGTTCACCGACTTTGACTCCCAAATCA
ATGTGCCGAAAACCATGGAATCTGGAACGTTCATCACTGACAAAACTGTG
CTGGACATGAAAGCTATGGATTCCAAGAGCAATGGGGCCATTGCCTGGAG
CAACCAGACAAGCTTCACCTGCCAAGATATCTTCAAAGAGACCAACGCCA
CCTACCCCAGTTCAGACGTTCCCTGTGATGCCACGTTGACCGAGAAAAGC
TTTGAAACAGATATGAACCTAAACTTTCAAAACCTGTCAGTTATGGGACT
CCGAATCCTCCTGCTGAAAGTAGCGGGATTTAACCTGCTCATGACGCTGA
GGCTGTGGTCCAGT,
and
(SEQ ID NO: 12; GenBank: U07662.1)
CCAAACATCCAGAACCCAGAACCTGCTGTGTACCAGTTAAAAGATCCTCG
GTCTCAGGACAGCACCCTCTGCCTGTTCACCGACTTTGACTCCCAAATCA
ATGTGCCGAAAACCATGGAATCTGGAACGTTCATCACTGACAAAACTGTG
CTGGACATGAAAGCTATGGATTCCAAGAGCAATGGGGCCATTGCCTGGAG
CAACCAGACAAGCTTCACCTGCCAAGATATCTTCAAAGAGACCAACGCCA
CCTACCCCAGTTCAGACGTTCCCTGTGATGCCACGTTGACCGAGAAAAGC
TTTGAAACAGATATGAACCTAAACTTTCAAAACCTGTCAGTTATGGGACT
CCGAATCCTCCTGCTGAAAGTAGCGGGATTTAACCTGCTCATGACGCTGA
GGCTGTGGTCCAGT.

TCR specific primers may be designed to specifically hybridize to a TCR beta chain (e.g., a TCR beta 1 chain constant region or a TCR beta 2 chain constant region) constant region or the complement thereof. For example, in some instances, a TCR specific primer may hybridize to a mammalian TCR beta chain constant region or a complement thereof, including e.g., a human TCR beta chain constant region, a mouse TCR beta chain constant region, Rhesus, hamster, camel, or the like.

An exemplary human TCR beta chain 1 constant region has the following amino acid sequence:

(SEQ ID NO: 13; UniProtKB: P01850;
GenBank: CAA25134.1)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF

which is encoded by the following nucleic acid sequence:

(SEQ ID NO: 14; GenBank: EF101778.1, X00437.1)
GAGGACCTGAACAAGGTGTTCCCACCCGAGGTCGCTGTGTTTGAGCCATC
AGAAGCAGAGATCTCCCACACCCAAAAGGCCACACTGGTGTGCCTGGCCA
CAGGCTTCTTCCCCGACCACGTGGAGCTGAGCTGGTGGGTGAATGGGAAG
GAGGTGCACAGTGGGGTCAGCACAGACCCGCAGCCCCTCAAGGAGCAGCC
CGCCCTCAATGACTCCAGATACTGCCTGAGCAGCCGCCTGAGGGTCTCGG
CCACCTTCTGGCAGAACCCCCGCAACCACTTCCGCTGTCAAGTCCAGTTC
TACGGGCTCTCGGAGAATGACGAGTGGACCCAGGATAGGGCCAAACCCGT
CACCCAGATCGTCAGCGCCGAGGCCTGGGGTAGAGCAGACTGTGGCTTTA
CCTCGGTGTCCTACCAGCAAGGGGTCCTGTCTGCCACCATCCTCTATGAG
ATCCTGCTAGGGAAGGCCACCCTGTATGCTGTGCTGGTCAGCGCCCTTGT
GTTGATGGCCATGGTCAAGAGAAAGGATTTC.

An exemplary human TCR beta chain 2 constant region has the following amino acid sequence:

(SEQ ID NO: 15; UniProtKB: A0A5B9,
GenBank: AAA60662.1)
DLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG
which is encoded by the following nucleic acid
sequence:
(SEQ ID NO: 16; GenBank: L34740.1)
GACCTGAAAAACGTGTTCCCACCCGAGGTCGCTGTGTTTGAGCCATCAGA
AGCAGAGATCTCCCACACCCAAAAGGCCACACTGGTATGCCTGGCCACAG
GCTTCTACCCCGACCACGTGGAGCTGAGCTGGTGGGTGAATGGGAAGGAG
GTGCACAGTGGGGTCAGCACAGACCCGCAGCCCCTCAAGGAGCAGCCCGC
CCTCAATGACTCCAGATACTGCCTGAGCAGCCGCCTGAGGGTCTCGGCCA
CCTTCTGGCAGAACCCCCGCAACCACTTCCGCTGTCAAGTCCAGTTCTAC
GGGCTCTCGGAGAATGACGAGTGGACCCAGGATAGGGCCAAACCCGTCAC
CCAGATCGTCAGCGCCGAGGCCTGGGGTAGAGCAGACTGTGGCTTCACCT
CCGAGTCTTACCAGCAAGGGGTCCTGTCTGCCACCATCCTCTATGAGATC
TTGCTAGGGAAGGCCACCTTGTATGCCGTGCTGGTCAGTGCCCTCGTGCT
GATGGCCATGGTCAAGAGAAAGGATTCCAGAGGCTAG.

An exemplary mouse TCR beta chain 1 constant region has the following amino acid sequence:

(SEQ ID NO: 17; UniProtKB: P01852)
EDLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS

which is encoded by the following nucleic acid sequence:

(SEQ ID NO: 18; GenBank: FJ188408.1)
GAGGATCTGAGAAATGTGACTCCACCCAAGGTCTCCTTGTTTGAGCCATC
AAAAGCAGAGATTGCAAACAAACAAAAGGCTACCCTCGTGTGCTTGGCCA
GGGGCTTCTTCCCTGACCACGTGGAGCTGAGCTGGTGGGTGAATGGCAAG
GAGGTCCACAGTGGGGTCAGCACGGACCCTCAGGCCTACAAGGAGAGCAA
TTATAGCTACTGCCTGAGCAGCCGCCTGAGGGTCTCTGCTACCTTCTGGC
ACAATCCTCGCAACCACTTCCGCTGCCAAGTGCAGTTCCATGGGCTTTCA
GAGGAGGACAAGTGGCCAGAGGGCTCACCCAAACCTGTCACACAGAACAT
CAGTGCAGAGGCCTGGGGCCGAGCAGACTGTGGGATTACCTCAGCATCCT
ATCAACAAGGGGTCTTGTCTGCCACCATCCTCTATGAGATCCTGCTAGGG
AAAGCCACCCTGTATGCTGTGCTTGTCAGTACACTGGTGGTGATGGCTAT
GGTCAAAAGAAAGAATTCATGA.

An exemplary mouse TCR beta chain 2 constant region has the following amino acid sequence:

(SEQ ID NO: 19; UniProtKB: P01851)
EDLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGLS
EEDKWPEGSPKPVIQNISAEAWGRADCGITSASYHQGVLSATILYEILLG
KATLYAVLVSGLVLMAMVKKKNS

which is encoded by the following nucleic acid sequence:

(SEQ ID NO: 20; GenBank: U46841.1)
GAGGATCTGAGAAATGTGACTCCACCCAAGGTCTCCTTGTTTGAGCCATC
AAAAGCAGAGATTGCAAACAAACAAAAGGCTACCCTCGTGTGCTTGGCCA
GGGGCTTCTTCCCTGACCACGTGGAGCTGAGCTGGTGGGTGAATGGCAAG
GAGGTCCACAGTGGGGTCAGCACGGACCCTCAGGCCTACAAGGAGAGCAA
TTATAGCTACTGCCTGAGCAGCCGCCTGAGGGTCTCTGCTACCTTCTGGC
ACAATCCTCGAAACCACTTCCGCTGCCAAGTGCAGTTCCATGGGCTTTCA
GAGGAGGACAAGTGGCCAGAGGGCTCACCCAAACCTGTCACACAGAACAT
CAGTGCAGAGGCCTGGGGCCGAGCAGACTGTGGAATCACTTCAGCATCCT
ATCATCAGGGGGTTCTGTCTGCAACCATCCTCTATGAGATCCTACTGGGG
AAGGCCACCCTATATGCTGTGCTGGTCAGTGGCCTGGTGCTGATGGCCAT
GGTCAAGAAAAAAAATTCCTGA.

In some instances, an immune cell receptor repertoire library may include a BCR repertoire library. The BCR complex is found on the surface of B cells and includes a membrane bound immunoglobulin (i.e., antibody) binding moiety, which includes a heavy and a light chain, each of which contains a constant (C) and a variable (V) region. The immunoglobulin chain of the BCR is bound by disulfide bridges to a signal transducing CD79A/B chains. The immunoglobulin chains of the BCR may be of various isotypes including IgD, IgM, IgA, IgG or IgE. Similar to the TCR, the immunoglobulin portion of the BCR undergoes V(D)J recombination to generate enormous diversity within a population.

In some instances, an immune cell receptor repertoire library may include a BCR repertoire library, where e.g., the BCR repertoire library may include BCR immunoglobulin chain sequences (including e.g., IgD, IgM, IgA, IgG or IgE chain sequences). Immunoglobulin chain sequences of a subject BCR repertoire library may include full length immunoglobulin chain sequences (e.g., full length heavy chain sequences, full length light chain sequences) or partial immunoglobulin sequences (e.g., partial heavy chain sequences, partial light chain sequences).

Where the subject BCR repertoire library members include partial immunoglobulin chain sequences, the partial immunoglobulin chain sequences may include the entire or essentially the entire immunoglobulin variable region (e.g., the immunoglobulin light chain variable region(s), the immunoglobulin heavy chain variable region(s)). In some instances, the resulting library members include the immunoglobulin variable region(s) and at least a portion of an immunoglobulin constant region. In some instances, the resulting library members include sequence corresponding to the immunoglobulin heavy and/or light chain 5′ mRNA ends. In some instances, the resulting library members include sequence from the immunoglobulin heavy or light chain 5′ end to at least a portion of the corresponding immunoglobulin chain constant region.

In certain embodiments, preparation of the immune cell specific library may include BCR specific amplification (including, e.g., immunoglobulin chain specific amplification). Such immunoglobulin specific amplification may make use of an immunoglobulin specific primer. By “immunoglobulin specific primer” is meant a primer that specifically hybridizes to a region of an immunoglobulin chain (e.g., a immunoglobulin heavy chain, an immunoglobulin light chain) nucleic acid sequence or the complement thereof. In some instances, an immunoglobulin specific primer may hybridize to only one type of immunoglobulin chain, e.g., only an immunoglobulin heavy chain, only an immunoglobulin light chain, only an IgD chain, only an IgM chain, only an IgA chain, only an IgG chain, only an IgE chain, etc.

Immunoglobulin specific primers may be designed to specifically hybridize to an immunoglobulin heavy chain constant region or the complement thereof. For example, in some instances, an immunoglobulin specific primer may hybridize to a mammalian immunoglobulin heavy chain constant region or a complement thereof, including e.g., a human immunoglobulin heavy chain constant region, a mouse immunoglobulin heavy chain constant region, or the like.

Immunoglobulin specific primers may be designed to specifically hybridize to an immunoglobulin light chain constant region or the complement thereof. For example, in some instances, an immunoglobulin specific primer may hybridize to a mammalian immunoglobulin light chain constant region or a complement thereof, including e.g., a human immunoglobulin light chain constant region, a mouse immunoglobulin light chain constant region, Rhesus, hamster, camel, or the like.

Amplification performed during library preparation, including e.g., immune receptor specific amplification, may be performed in a single round or multiple rounds of amplification may be employed. For example, in some instances, after a first round of amplification one or more amplification primers not utilized in the first round may be added to the reaction mixture to facilitate a second round of amplification using the product of the first round of amplification as a nucleic acid template. In some instances, the second or subsequent round(s) of amplification may involve nested amplification, i.e., where the primer binding sites utilized in the second or subsequent round(s) of amplification are within (i.e., one or more nucleotides from the 3′ or 5′ end) of the product generated in the first round of amplification. Where employed, the degree of nesting will vary as desired including e.g., where the second or subsequent primer binding site is one or more, including 2 or more, 3 or more, 4 or more, 5 or more, 6 or more, 7 or more, 8 or more, 9 or more, 10 or more, 15 or more, 20 or more, etc., nucleotides from the 3′ or 5′ end of the amplicon generated in the first round of amplification.

In some instances, second or subsequent round(s) amplification will not be nested, including where the second round of amplification makes use of one or more primer binding sites utilized in the prior round of amplification or a primer binding site added during the prior round of amplification (e.g., a primer binding site added as part of a non-templated sequence). In some instances, a second or subsequent round of amplification may make use of a nested primer amplification site at one end and a non-nested (e.g., a prior used primer binding site or an added primer binding site) at the other end, including where the nested site is at the 3′ end of the amplicon or the 5′ end of the amplicon.

### Kits, Compositions and Devices

Aspects of the present disclosure also include compositions and kits as well as devices for use therewith or therein.

Most generally, the term “kit” is used to describe any assemblage of articles that facilitate the execution of a process, method, assay, analysis, manipulation of a sample, or the like. Kits can contain written instructions describing how to use the kit (e.g., instructions describing the methods of the present invention), chemical reagents or enzymes required for the method, primers, probes, buffer solutions, any type of containers (for example, containers for sample collection or sample manipulation) or reaction vessels, or any other components. A kit need not contain every component necessary to execute a method of the invention. The compositions and kits of the invention may include, e.g., one or more of any of the reaction components described above with respect to the subject methods.

In some embodiments, kits of the invention may include a plurality of separate template switch oligonucleotide compositions each comprising template switch oligonucleotides that include a common first identifier, wherein the first identifiers of template switch oligonucleotides of different template switch oligonucleotide compositions are different; and a plurality of separate second identifier nucleic acids, e.g., which may be provided as sub-domains. In such instances, a given template switch oligonucleotide composition may be made of many copies of the same template switch oligonucleotide, or a population of different template switch oligonucleotides that share the same or common first identifier sequence but also differ from each with respect to UMI domains. Where desired, the different template switch oligonucleotides may be present in different containers, e.g., in different wells of a multi-plate, including different microwells of a microwell plate. As with the template switch oligonucleotide compositions, the different second identifier nucleic acids may be present in separate containers, e.g., in different wells of a multi-plate, including different microwells of a microwell plate, where the separate containers are distinct from the containers holding the template switch oligonucleotide compositions.

The kits may further include one or more additional reagents employed in embodiments of the invention, e.g., as described above, where such reagents may include, but are not limited to: one or more polymerases (e.g., a template switching polymerase, a reverse transcriptase, an amplification polymerase, etc.), ligases, transposases, primers, buffers, dNTPs (including e.g., dATP, dCTP, dGTP, dTTP, dUTP, etc. or any one or any combination thereof), and the like. The subject kits may include, or the compositions and devices may be provided with, one or more test reagents, including e.g., control nucleic acids (e.g., control nucleic acid templates), and the like. In some instances, the reagents may be provided in lyophilized form, such as lyophilized enzymes, e.g., lyophilized reverse transcriptase, lyophilized DNA polymerase, etc.

In some instances, components of the subject compositions and/or kits may be presented as a “cocktail” where, as used herein, a cocktail refers to a collection or combination of two or more different but similar components in a single vessel. Components of the kits may be present in separate containers, or multiple components may be present in a single container, as desired. The subject compositions may be present in any suitable environment. According to one embodiment, the composition is present in a reaction tube (e.g., a 0.2 mL tube, a 0.6 mL tube, a 1.5 mL tube, or the like) or a well or microfluidic chamber or droplet or other suitable container. In certain aspects, the composition is present in two or more (e.g., a plurality of) reaction tubes or wells (e.g., a plate, such as a 96-well plate, a multi-well plate, e.g., containing about 1000, 5000, or 10,000 or more wells). The tubes and/or plates may be made of any suitable material, e.g., polypropylene, or the like, PDMS, or aluminum. The containers may also be treated to reduce adsorption of nucleic acids to the walls of the container. In certain aspects, the tubes and/or plates in which the composition is present provide for efficient heat transfer to the composition (e.g., when placed in a heat block, water bath, thermocycler, and/or the like), so that the temperature of the composition may be altered within a short period of time, e.g., as necessary for a particular enzymatic reaction to occur. According to certain embodiments, the composition is present in a thin-walled polypropylene tube, or a plate having thin-walled polypropylene wells or materials such as aluminum having high heat conductance.

In some instances, a collection of individual vessels (e.g., separate tubes) or containing multiple vessels, e.g., a multi-well device may include reagents, which may be provided in liquid or dried form.

Any suitable reaction vessel(s) may be employed in the subject kits or devices and/or to contain a subject composition. Useful reaction vessels include but are not limited to e.g., tubes (e.g., single tubes, multi-tube strips, etc.), wells (e.g., of a multi-well plate (e.g., a 96-well plate, 384 well plate, or a plate with any number of wells such as 2000, 4000, 6000, or 10000 or more). Multi-well plates may be independent or may be part of a chip and/or device, e.g., as described in greater detail below. As such, in certain embodiments, the reaction vessel employed is a well or wells of a multi-well device. The present disclosure is not limited by the type of multi-well devices (e.g., plates or chips) employed. In general, such devices have a plurality of wells that contain, or are dimensioned to contain, liquid (e.g., liquid that is trapped in the wells such that gravity alone cannot make the liquid flow out of the wells). One exemplary chip is the 5184-well SMARTCHIP™ (Takara Bio USA, San Jose CA). Other exemplary chips are provided in U.S. Pat. Nos. 8,252,581; 7,833,709; and 7,547,556, all of which are herein incorporated by reference in their entireties including, for example, for the teaching of chips, wells, thermocycling conditions, and associated reagents used therein). Other exemplary chips include the OPENARRAY™ plates used in the QUANTSTUDIO™ real-time PCR system (sold by Applied Biosystems). Another exemplary multi-well device is a 96-well or 384-well plate.

In addition to the above-mentioned components, a subject kit may further include instructions for using the components of the kit, e.g., to practice the subject methods as described above. The instructions are generally recorded on a suitable recording medium. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, CD-ROM, diskette, Hard Disk Drive (HDD) etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The following example(s) is/are offered by way of illustration and not by way of limitation.

### EXAMPLES

### Example 1. Analysis of Expression of a Gene or Genes in Each of a Plurality of Cells or Nuclei

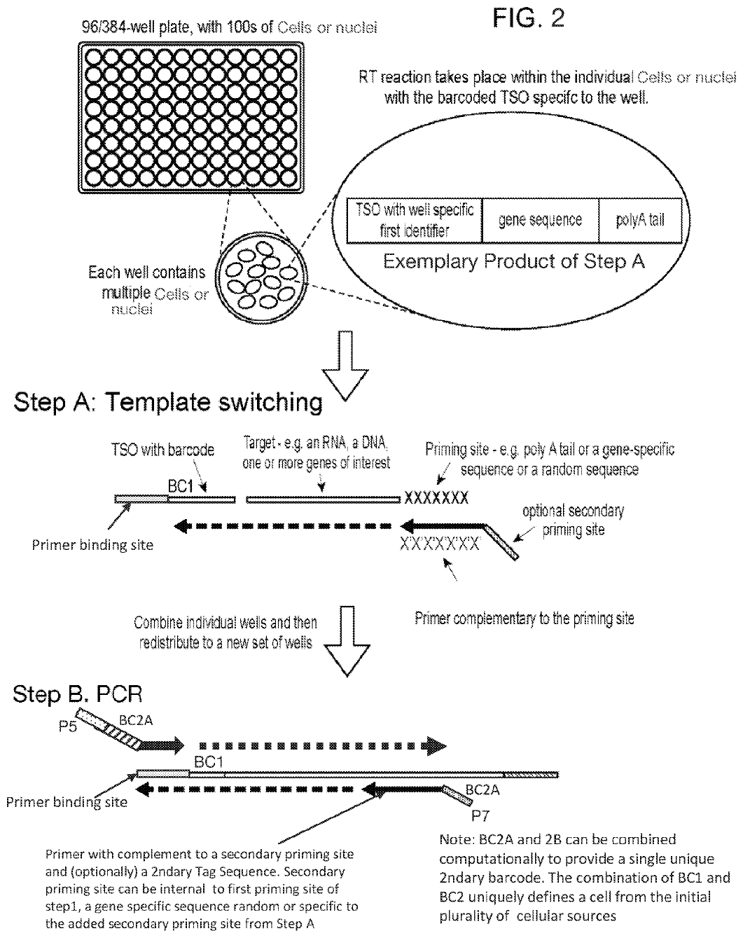

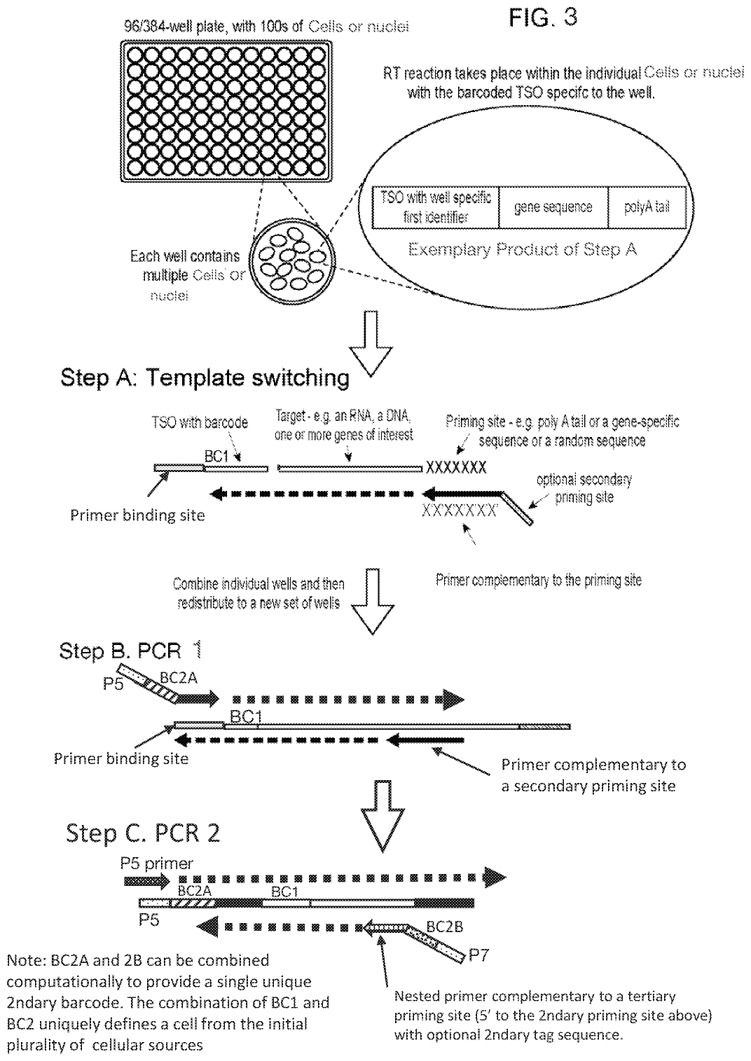

This example is broadly depicted in FIGS. 2 and 3. Cells or nuclei are fixed and permeabilized in an appropriate solution (e.g., 1% or 4% paraformaldehyde with NP40 or digitonin). The fixed cells or nuclei are aliquoted to the wells of a multi-well device (for example a 96 well or 384 well plate). Alternatively, the wells need not be physical wells, but could be comprised of droplets, and the cells or nuclei apportioned to different droplets. The number of cells or nuclei per well or container may vary as appropriate for the scale of the experiment that the researcher wishes to perform. For example, 100 or 1,000 cells per well for each of the 96 or 384 wells of the plate can be analyzed. Reverse transcription mix comprising reverse transcriptase, oligo dT, template switch oligo (TSO) comprising a first cell-specific barcode (i.e., a well-specific first identifier or first index) and an adapter handle (i.e., primer binding site) for PCR, dNTPs and buffer salts are added to each of the wells, and the RT reaction allowed to proceed (for example at any appropriate temperature such as 37-50° C., e.g., at 42° C. for sufficient time to complete the reaction, such as 60-90 mins or more). This is depicted in Step A of FIG. 2. The reaction is stopped, and the cells or nuclei collected from each of the wells, pooled together and then redistributed to the wells of a second multi well device. Alternatively, the cells or nuclei can be redistributed to another set of droplets. Optionally, lysis buffer is added to each of the wells to release the nucleic acid from each of the cells or nuclei. Optionally, the nucleic acids are purified in each well independently. Reagents effective for performing PCR are then added to the wells. These may include thermal stable polymerase, dNTPs, buffer and two or more PCR primers. The first primer being specific for the adapter handle (primer binding site) sequence present in the TSO used in the reverse transcriptase step and one or more second primers that have at their 3′ end a region complementary to either a gene specific sequence (e.g., a TCR constant region gene sequence), a poly A sequence, an adapter handle, or a random sequence, as shown in step B of FIGS. 2. In one embodiment, each of the two or more PCR primers additionally contains a barcode sequence (shown as BC2A and BC2B in FIG. 2), which when combined provide a second cell-specific barcode sequence (i.e., a second identifier tag). In an alternative embodiment, BC2A or BC2B can be used alone as a second cell-specific barcode sequence (i.e., a second identifier tag). Optionally, the PCR primers may also comprise additional sequences for next generation sequencing. For example, sequencing platform adapter constructs such as read primer sequences, p5, p7 sequences, and the like. Alternatively, these sequencing platform adapter construct sequences can be added in a second round of PCR. This alternative embodiment with a second round of PCR is depicted in FIG. 3, where the second PCR (PCR 2) is shown as additional Step C. In this step C, a nested 3′ primer is used internally to the secondary primer binding site from Step B. This nested primer includes sequence complementary to a tertiary priming site (5′ to the secondary priming site of Step B), an optional secondary tag sequence (second sub-identifier, BC2B), and the Illumina p7 sequence.

As depicted in FIGS. 2 and 3, the second identifier tag is added in two parts (BC2A and BC2B). In the embodiment shown in FIG. 2, BC2A and BC2B are both added as part of PCR 1 (Step B of FIG. 2). In the embodiment shown in FIG. 3, BC2A is added in step B of FIG. 3 and BC2B is added as in step C.

The resulting sequencing products are then sequenced using a next generation sequencing platform to obtain gene sequence information and barcode information—a combination of the first (TSO) barcode (i.e., first identifier) and second (combined PCR barcodes; 2^{nd} identifier) providing a unique cell-specific barcode that can trace the gene specific sequence to each individual cell or nucleus. Thus, the expression of the gene or genes detected in each of the cells or nuclei of the original collection is determined.

### Example 2. An Embodiment of the Invention

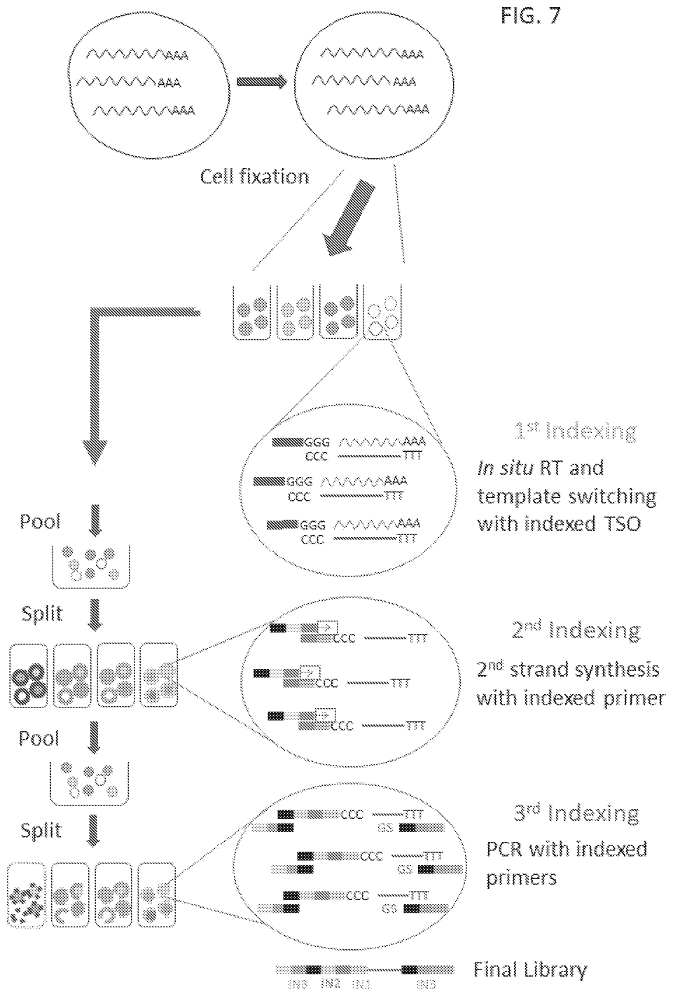

This example follows the same initial steps as EXAMPLE 1, up to the redistribution of the cells or nuclei into the second multi-well device. This example is broadly depicted in FIG. 7.

Cells or nuclei to be analyzed are fixed and permeabilized in an appropriate solution (e.g., 1% or 4% paraformaldehyde with NP40 or digitonin). The fixed cells or nuclei are aliquoted to the wells of a multi-well device (for example a 96 well or 384 well plate). Alternatively, in place of physical wells, the cells or nuclei can be apportioned to a plurality of droplets, as known in the art.

The number of cells or nuclei per well or container may vary as appropriate for the scale of the experiment that the researcher wishes to perform. For example, 100 or 1,000 cells per well for each of the 96 or 384 wells of the plate can be used. Reverse transcription mix comprising reverse transcriptase, oligo dT, template switch oligo (TSO) comprising a first cell-specific barcode (i.e., well-specific first identifier tag) and an adapter handle (i.e., primer binding site) for PCR, dNTPs and buffer salts are added to each of the wells and the RT reaction allowed to proceed (for example, at any appropriate temperature such as 37-50° C., e.g., at 42° C. for sufficient time to complete the reaction, such as 60-90 mins or longer). The reaction is stopped, and the cells or nuclei collected from each of the wells, pooled together and then redistributed to the wells of a second multi well device. Alternatively, the cells or nuclei may be redistributed to another set of droplets.

Second strand synthesis and/or iso-thermal amplification is performed on the redistributed cells or nuclei. At this stage, the redistributed material still remains as intact cells or intact nuclei in the individual wells of the second multi-well device. Second strand synthesis is initiated by adding one or more of the following to the wells: reaction buffer, dNTPs, second strand primer, e.g., a primer comprising a second well-specific barcode (i.e., a second identifier tag) and a second primer binding site, and a polymerase. Second strand synthesis is performed to add the second barcode 5′ to the TSO barcode (i.e., well-specific first identifier tag). Optionally, iso-thermal amplification is performed by including one or more reverse primers that have at their 3′ end either a target-specific sequence, an oligo dT sequence, a sequence complementary to an adapter handle sequence added in the first round, or a random sequence, thereby generating cell-specific nucleic acids that have two barcodes—one from each step (the template switching step and the second strand synthesis step), which when combined can identify which well of the first multi-well device and which well of the second multi-well device any particular cell or nucleus was originally located.

After completion of this second barcoding step, the cells are again collected from each of the wells of the second plate, pooled together and redistributed to a 3^{rd} multi-well device. Optionally, the cells or nuclei are lysed and nucleic acids are purified in each well independently. Reagents for PCR are then added to the wells including thermal stable polymerase, dNTPs, buffer and two PCR primers. The first primer being specific for the adapter handle sequence (second primer binding) present in the primer used in the second strand synthesis step and one or more second primers that have at their 3′ end a region complementary to either a gene specific sequence, nested internally to the reverse primer used in the second step described above (e.g., a TCR constant region gene sequence), or an adapter handle that was included in the reverse primer used in the second indexing step. One or both of these PCR primers may contain a barcode sequence (i.e., a third identifier tag), which either alone or combined provide a 3rd cell-specific barcode sequence.

Optionally, the PCR primers may also comprise additional sequences for next generation sequencing. For example, sequencing platform adapter constructs such as read primer sequences, p5, p7 sequences, and the like. Alternatively, these sequencing platform adapter construct sequences can be added in another round of PCR following the PCR reaction used to add the 3^{rd} identifier sequences (3rd cell-specific barcode sequence).

The resulting sequencing products are then sequenced using a next generation sequencing platform to obtain gene sequence information and barcode information. The combination of the barcodes from each round of indexing addition identifies the cell or nucleus from which any individual cell or nucleus sequence came by virtue of the cell or nucleus' unique passage through first, second and third unique wells of each respective multi-well device. That is, for example, the first (TSO) barcode second barcode (from second strand synthesis) and 3^{rd} barcode from PCR providing a unique cell-specific barcode that can trace the gene specific sequence to each individual cell or nucleus. The expression of the gene or genes detected in each of the cells or nuclei of the original collection is thus determined.

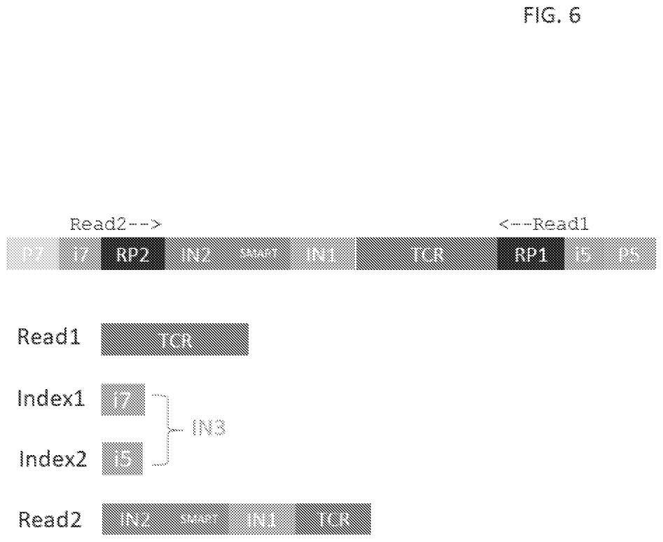

FIG. 6 provides a schematic showing the structure of an NGS library product made using an embodiment of the invention, as described in Example 2. More particularly, T cell receptor genes are specifically targeted for analysis. As shown in FIG. 6, Read 1 provides sequence of the targeted TCR gene. Read 2 also provides the sequence of T cell receptor genes together with the first two index sequences, namely: index 2 (IN2) from the second indexing step, and index 1 (IN1) from the 1st indexing step. Index 3 is shown as being provided by the combination of the i7 and i5 indexes added by PCR in the 3rd indexing step.

### Example 3. An Embodiment of the Invention

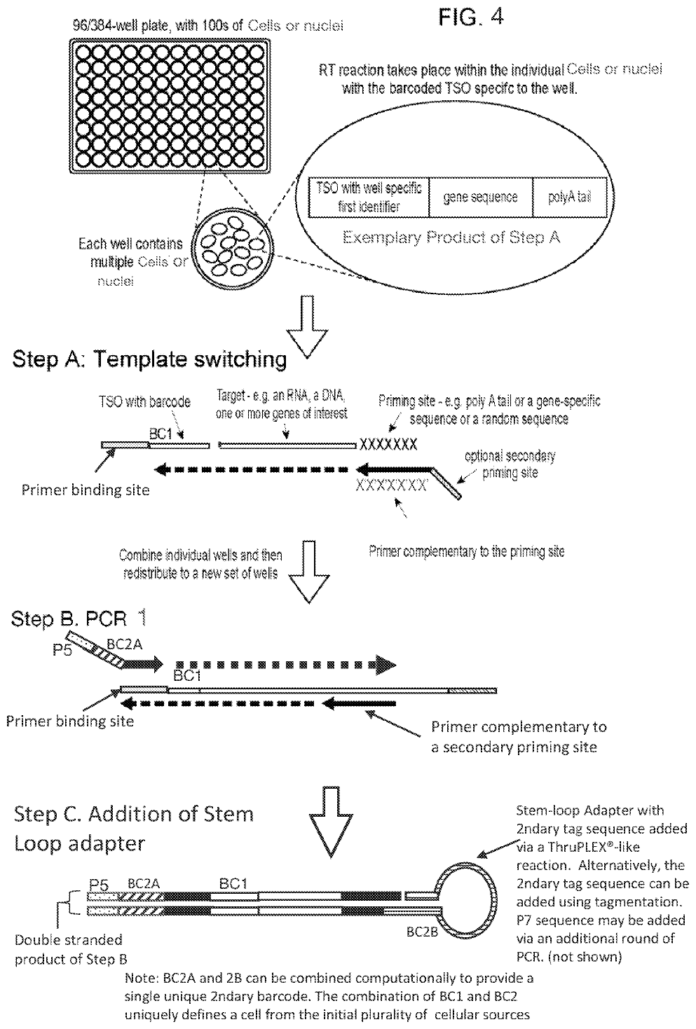

EXAMPLE 3 is broadly depicted in FIG. 4 and is performed in a manner similar to EXAMPLE 1, but differs in how the 3′ barcode is added in a second step, as described below. This example follows the same initial steps as EXAMPLE 1, resulting in the addition of the first cell-specific barcode (i.e., well specific first identifier tag). The steps of the methodology are described below.

Cells or nuclei are fixed and permeabilized in an appropriate solution (e.g., 1% or 4% paraformaldehyde with NP40 or digitonin). The fixed cells or nuclei are aliquoted to the wells of a multi-well device (for example a 96 well or 384 well plate). Alternatively, the wells need not be physical, but could be comprised of droplets and the cells or nuclei apportioned to different droplets. The number of cells or nuclei per well or container may vary as appropriate for the scale of the experiment that the researcher wishes to perform. For example, 100 or 1,000 cells per well for each of the 96 or 384 wells of the plate. Reverse transcription mix comprising reverse transcriptase, oligo dT, template switch oligo (TSO) comprising a first cell-specific barcode (i.e., well specific first identifier tag) and an adapter handle (i.e., primer binding site) for PCR, dNTPs and buffer salts are added to each of the wells and the RT reaction allowed to proceed (for example, at any appropriate temperature such as 37-50° C., e.g., at 42° C., for sufficient time to complete the reaction, such as 60-90 minutes or longer). This is depicted in Step A of FIG. 4. The reaction is stopped, and the cells or nuclei are collected from each of the wells, pooled together and then redistributed to the wells of a second multi well device. Alternatively, the cells or nuclei may be redistributed to another set of droplets.

If desired, lysis buffer is added to each of the wells to release the nucleic acid from each of the cells or nuclei. Optionally, the nucleic acids are purified in each well independently. Reagents effective for performing PCR are then added to the wells, as depicted in Step B of FIG. 4—PCR1. These may include thermal stable polymerase, dNTPs, buffer and two or more PCR primers. The first primer being specific for the adapter handle (primer binding site) sequence present in the TSO used in the reverse transcriptase step and one or more second primers that have at their 3′ end a region complementary to either a gene specific sequence (e.g., a TCR constant region gene sequence), a poly A sequence, and adapter handle, or a random sequence. This is shown as Step B in FIG. 4. In this step, all or a portion of a second cell specific barcode sequence (second identifier) is included in the primer that binds to the primer binding site in the TSO. In FIG. 4, this is shown as BC2A. After amplification, a hairpin adapter comprising all or a sub-portion of a second cell specific barcode is added to the 3′ end of the fragments generated in Step B using a modified version of a ThruPLEX® library preparation kit (Takara Bio USA Inc., San Jose, CA), i.e., using only a single adapter, so that an adapter with barcode is added to the 3′ end of the fragments. This is detailed in Step C of FIG. 4.

Optionally, Tagmentation may be used instead of ThruPLEX® to add the barcode adapter to the end of the fragments. Following addition of this adapter, PCR is performed to amplify only sequences that have the adapter on the 3′ end and add any additional sequences required for next generation sequence such as, for example, the Illumina P7 sequence. If desired, for example to reduce the total number of barcoded oligos required, the primer specific to the handle in the template switch oligo can include a sub-portion of the second cell-specific barcode (i.e., second identifier tag). This sub identifier (BC2A in FIG. 4), when combined with the sub identifier provided by the tag in the 3′ adapter (BC2B in FIG. 4), provides a second cell-specific barcode sequence (i.e., second identifier tag). It should be understood by those of skill in the art that either BC2A or BC2B could be used as the entire second identifier tag without the need to use the other tag or that a combination of BC2A and BC2B can be used to provide a combined second identifier.

The resulting sequencing products are then sequenced using a next generation sequencing platform to obtain gene sequence information and barcode information—a combination of the first (TSO) barcode, second barcode (from second strand synthesis) and third barcode from PCR providing a unique cell-specific barcode that can trace the gene specific sequence to each individual cell or nucleus. The expression of the gene or genes detected in each of the cells or nuclei of the original collection is thus determined.

As illustrated in FIGS. 2, 3 and 4, the barcodes BC2A and 2B can be combined computationally to be a single unique secondary barcode that uniquely defines the well of the second pool step. Combining BC1 and BC2 uniquely defines a cell from the original pool.

### Example 4. Two-Round Barcoding of TCR Beta Chains from a Mixture of Jurkat and CCRF-CEM Cells

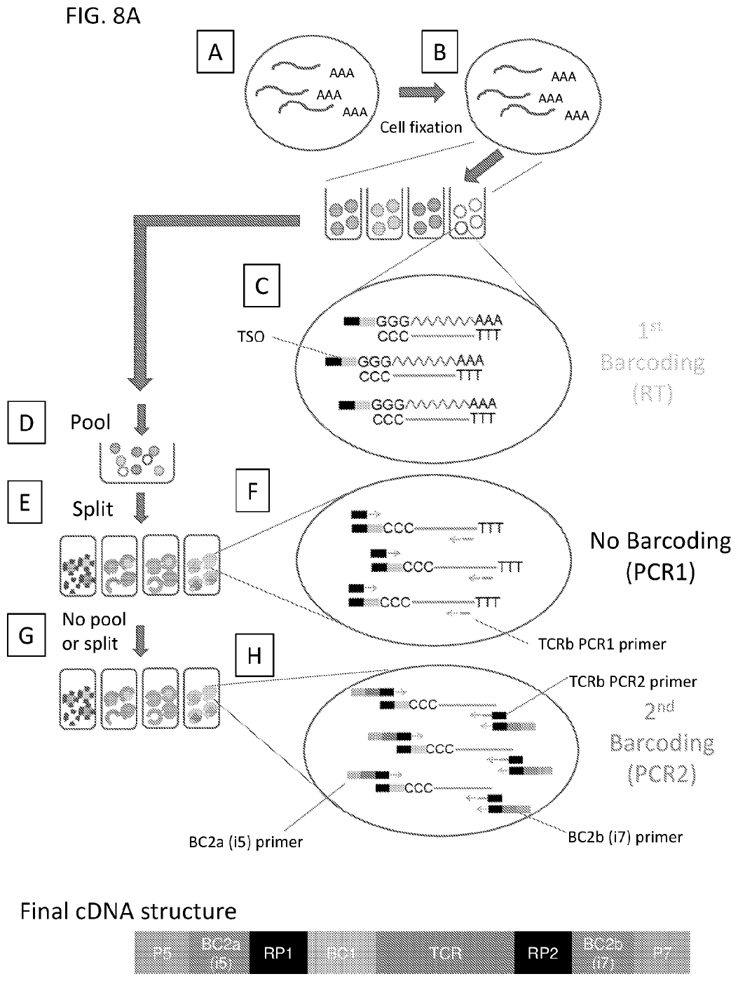

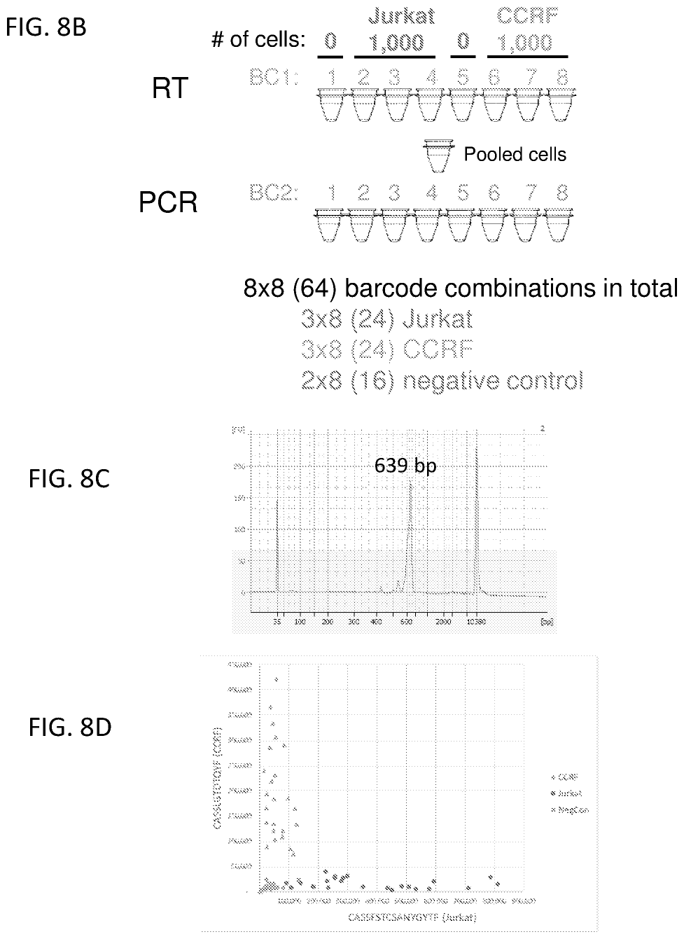

FIGS. 8A & B illustrate a two-round barcoding protocol that was employed to prepare a TCR sequencing library. As illustrated in FIG. 8A, Jurkat and CCRF-CEM cells were fixed by incubation at −20° C. for 30 min with 4 volumes of cold methanol (FIG. 8A, Step B). The fixed cells were removed from the −20° C. freezer and, after removal of the methanol, rehydrated on ice with 500 ul of rehydration buffer comprising PBS buffer, BSA, RNase Inhibitor and DTT. 1,000 fixed Jurkat and CCRF-CEM cells were distributed to each of 3 tubes as well as 2 tubes with PBS as negative control (FIG. 8B). Reverse transcription mix comprising reverse transcriptase, RNase inhibitor, poly-dT oligo, template switch oligo (TSO) comprising a first tube-specific barcode (BC1) and an Illumina RP1 sequence, dNTPs and RT buffer, were added to each of the tubes, and the RT reaction was performed at 42° C. for 90 minutes (FIG. 8A, Step C). The cells were collected from each of the tubes and pooled together (FIG. 8A, Step D). After centrifugation, the supernatant was discarded, and the cells were resuspended with PBS buffer. The resuspended cells were redistributed to a new set of 8 tubes (FIG. 8A, Step E and FIG. 8B).

PCR1 mix comprising DNA polymerase, a PCR primer comprising an Illumina RP1 sequence; a primer that specifically hybridizes to the constant region of the T-cell antigen receptor (TCR) beta chain (TCRb PCR1 primer); dNTPs and PCR buffer, was added to 8 tubes containing resuspended cells. PCR1 was then performed in 40 ul (FIG. 8A, Step F). The TCRb PCR1 primer is a chimeric DNA/RNA oligonucleotide that works as PCR primer in the absence of RNase, but can be made inactive after PCR by digestion by various RNases (e.g., RNaseH, RNaseA, etc.), e.g., as described in U.S. patent application Ser. No. 16/603,788 published as US 2020-0332341 A1 (Attorney Docket No. CLON-169), the disclosure of which is herein incorporated by reference.

PCR2 mix comprising DNA polymerase; RNaseH; BC2a (i5) primer comprising an Illumina RP1 sequence, BC2a (i5) and the P5 adapter sequence; BC2b (i7) primer comprising an Illumina RP2 sequence, BC2b (i7) and the P7 adapter sequence; TCRb PCR2 primer, which hybridizes to the TCRb constant region at a point internal to the TCRb PCR1 primer, and additionally comprises an Illumina RP2 sequence; dNTPs; and PCR buffer, was directly added to the 8 tubes containing the PCR1 reaction product. PCR2 was then performed in 70 ul (FIG. 8A, Step H). The resultant TCRb library contains all molecules marked with both first and second round barcodes (BC1, BC2a (i5) and BC2b (i7)).

The 8 barcoded TCRb libraries were purified with magnetic beads, quantified by Qubit, Bioanalyzer High sensitivity kit (one of 8 libraries is shown in FIG. 8C) and qPCR. They were then pooled together and loaded on a NextSeq sequencer (Illumina Inc., San Diego CA) for paired-end sequencing (2×151 PE). The resultant sequencing reads were demultiplexed with BC1, BC2a (i5) and BC2b (i7) and analyzed by Cogent AP software (Takara Bio USA, Inc., San Jose CA). All 64 expected barcode combinations (8 first round barcodes×8 second round barcodes) were detected, and the results plotted based on the number of reads of Jurkat clonotype (amino acid sequence: CASSFSTCSANYGYTF) and CCRF clonotype (amino acid sequence: CASSLGTDTQYF) detected (FIG. 8D). As expected, most of reads of were either assigned to the Jurkat or CCRF clonotype. This confirmed that the combinatorial barcoding strategy, including use of a first-round barcode from the TSO was working as expected.

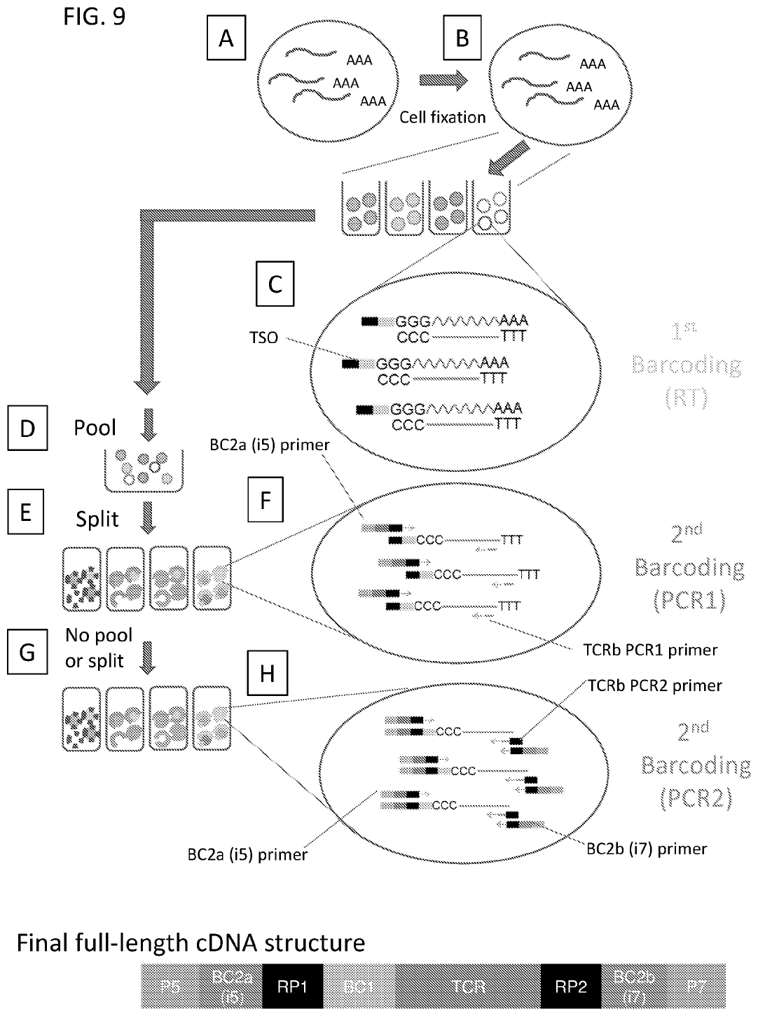

Note an alternative barcoding strategy is also envisioned wherein the forward primer for PCR1 includes partial second round barcode BC2a, a P5 sequence and the Illumina RP1 sequence. This is shown in FIG. 9.

### Example 5. Three-Round Barcoding of TCR Alpha and Beta Chains from PBMC RNA

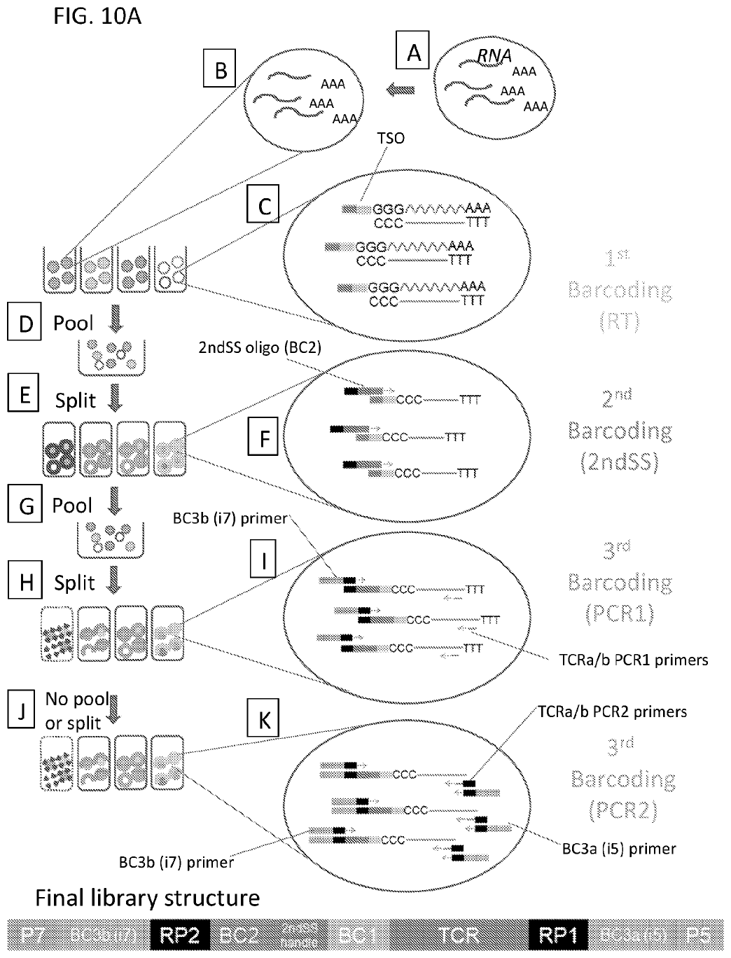

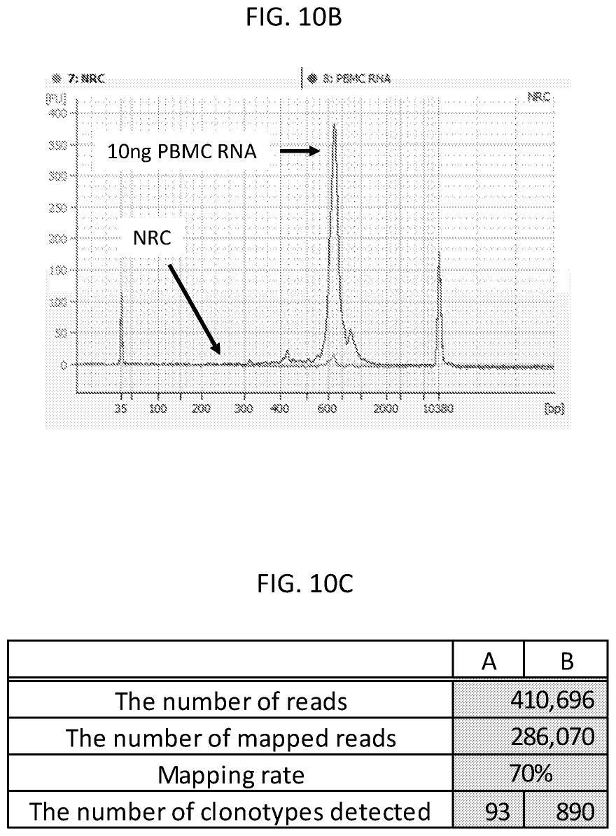

A sequencing library was prepared using a three-round barcoding protocol as illustrated in FIG. 10A. PBMC RNA (Takara Bio USA Inc.) was diluted to 5 ng/ul and 2 ul (10 ng) was distributed to an eppendorf tube. As a negative control, 2 ul RNase free water was distributed to another tube. Reverse transcription mix comprising reverse transcriptase, RNase inhibitor, poly-dT oligo, template switch oligo (TSO) comprising a first barcode (BC1) and a primer binding site for second strand synthesis (2ndSS handle), dNTPs and RT buffer, was added to each tube, and the RT reaction was performed in 20 ul at 42° C. for 90 minutes, followed by incubation at 70° C. for 10 minutes (FIG. 10A, Step C). The TSO used in this reaction is a chimeric DNA/RNA oligonucleotide that works as template switching oligo in the absence of RNase, but can be inactivated after use by digestion with various RNases (e.g., RNaseH, RNaseA, etc.), e.g., as described in U.S. patent application Ser. No. 16/603,788 published as US 2020-0332341 A1 (Attorney Docket No. CLON-169), the disclosure of which is herein incorporated by reference. The RT product with BC1 was purified by magnetic beads and eluted into 13 ul elution buffer.

2nd strand synthesis mix (2ndSS) comprising reverse transcriptase; RNaseH; 2ndSS oligo comprising a sequence that hybridizes to the TSO primer binding sequence, BC2 and the Illumina RP2 sequence; dNTPs and 2ndSS reaction buffer, was added to a clean tube containing 13 ul of purified RT product. The 2ndSS reaction was then performed in 20 ul at 42 C for 10 min and followed by incubation at 70° C. for 10 minutes (FIG. 10A, Step F). The 2ndSS product with BC1 and BC2 was purified by magnetic beads and eluted into 12 ul elution buffer.

PCR1 mix comprising DNA polymerase; BC3b (i7) primer comprising an Illumina RP2 sequence, BC3b (i7) and the P7 adapter sequence; TCRa PCR1 primer, which specifically hybridizes to the constant region of the T-cell antigen receptor (TCR) alpha chain; TCRb PCR1 primer, which specifically hybridizes to the constant region of the T-cell antigen receptor (TCR) beta chain; dNTPs and PCR buffer, was added to a clean tube containing 10 ul of purified 2ndSS product, and then PCR1 was performed in 40 ul (FIG. 10A, Step I). The TCRa PCR1 primer and TCRb PCR1 primer above are both chimeric DNA/RNA oligonucleotides that can work as PCR primers in the absence of RNase, but which can be inactivated by digestion with various RNases (e.g., RNaseH, RNaseA, etc.) after use, e.g., as described in U.S. patent application Ser. No. 16/603,788 published as US 2020-0332341 A1 (Attorney Docket No. CLON-169), the disclosure of which is herein incorporated by reference.

Following PCR1, PCR2 mix comprising DNA polymerase; RNaseH; BC3a (i5) primer comprising an Illumina RP1 sequence, BC3a (i5) and the P5 adapter sequence; TCRa PCR2 primer, which hybridizes to the TCRa constant region at a point internal to the TCRa PCR1 primer, and additionally comprises an Illumina RP1 sequence; TCRb PCR2 primer, which hybridizes to the TCRb constant region at a point internal to the TCRb PCR1 primer, and additionally comprises an Illumina RP1 sequence; dNTPs and PCR buffer, was directly added to the PCR1 tube. PCR2 was then performed in 70 ul (FIG. 10A, Step K). After PCR2, the TCR library comprising molecules marked with first, second and third round barcodes (BC1, BC2, BC3a (i5) and BC3b (i7)) was purified by magnetic beads and quantified by Qubit and Bioanalyzer High sensitivity kit. The library, obtained from only from 10ng PBMC RNA, was conformed to have the expected size (˜650 bp) (FIG. 10 Panel B).

The library was loaded on to an Illumina MiSeq (Illumina Inc, San Diego, CA) for paired-end sequencing (2×151 PE), and the resultant sequencing data analyzed using Cogent AP software (Takara Bio USA, Inc. San Jose CA). 410,696 reads were obtained after demultiplexing and 286,070 reads, representing a mapping rate of 70%, were mapped to TCRa and TCRb. The number of clonotypes detected was 93 (TCRa) and 890 (TCRb) (FIG. 10 Panel C). This result demonstrates that a three-round combinatorial barcoding strategy using a TSO to supply the first-round barcode (first identifier) and second strand synthesis to provide the second-round barcode (second identifier) works as expected.

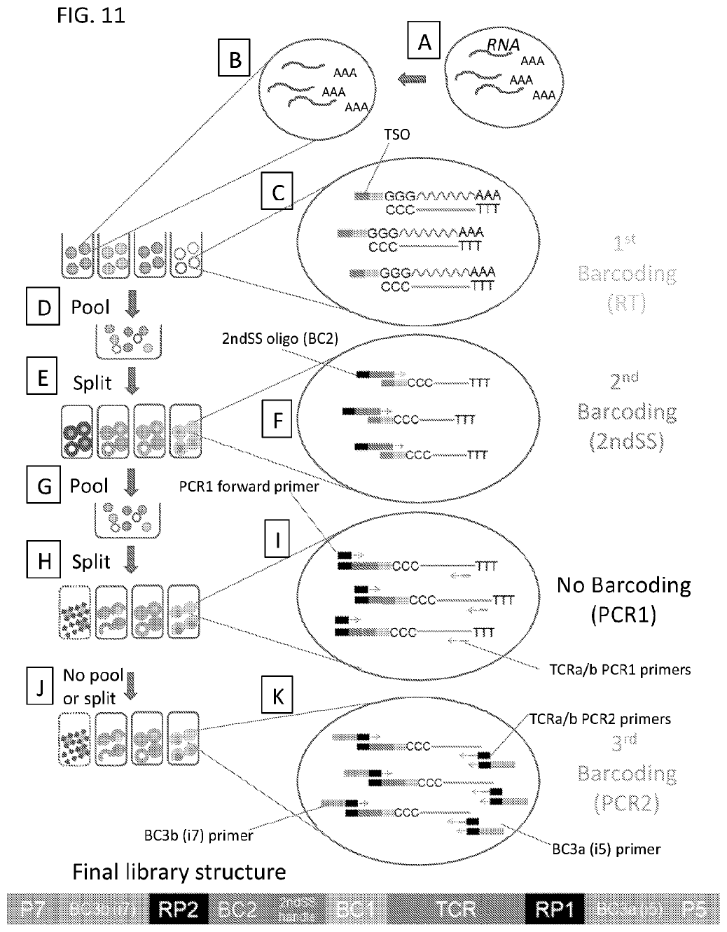

Note an alternative barcoding strategy is also envisioned wherein the forward primer for PCR1 does not include a barcode sequence. This is shown in FIG. 11.

### Example 6. Two-Round Barcoding for Combined Targeted Sequencing and 5′ Differential Expression (5′DE)

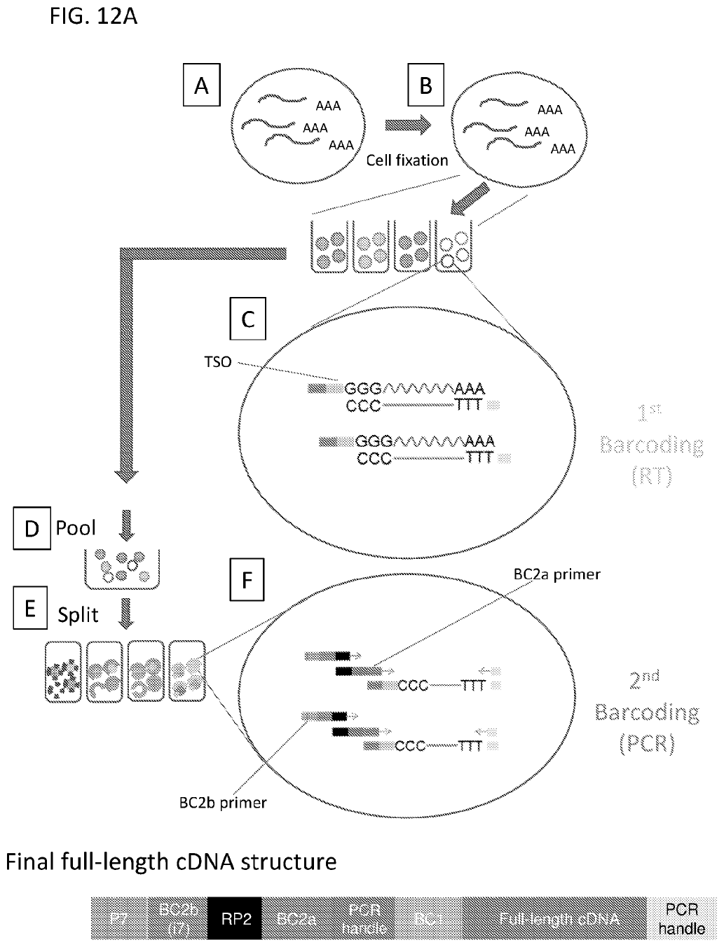

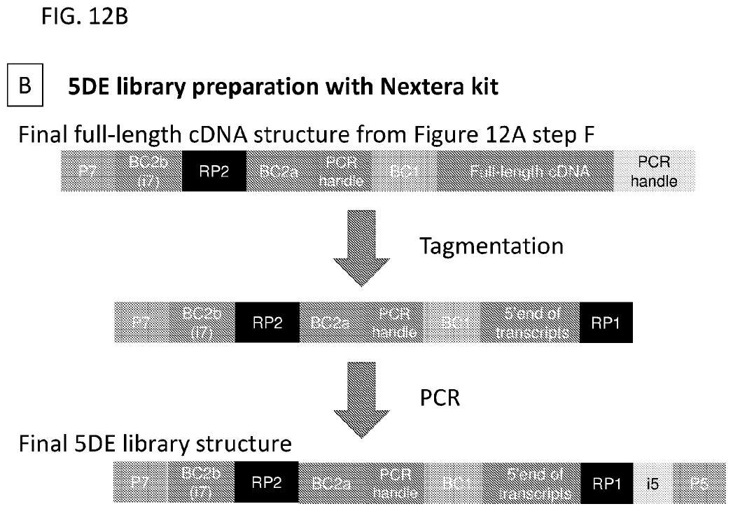

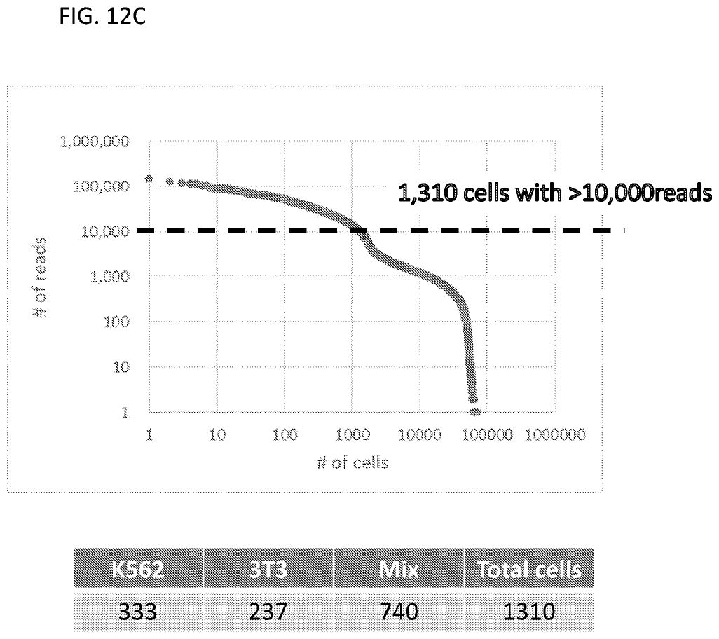

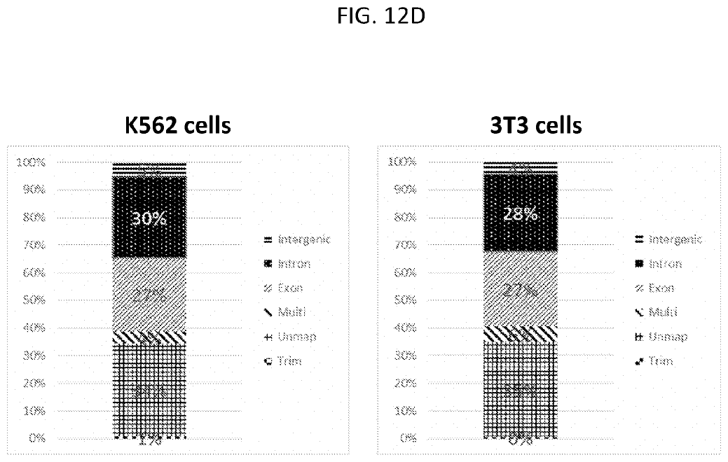

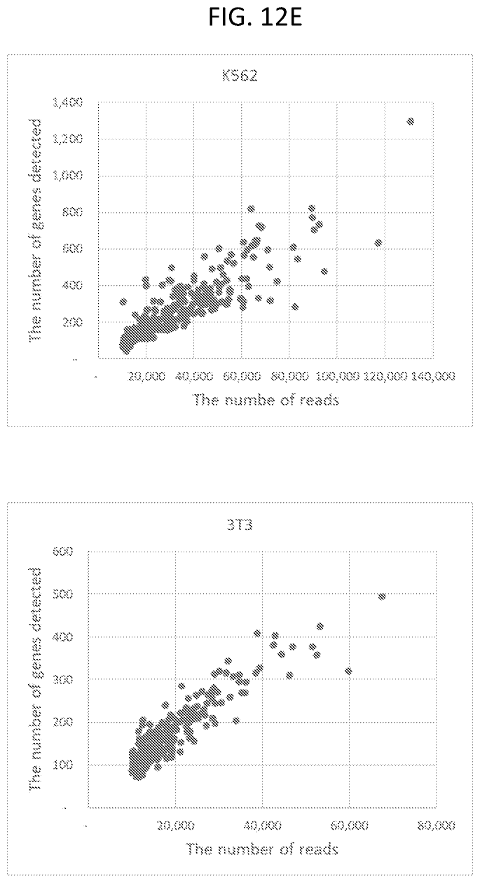

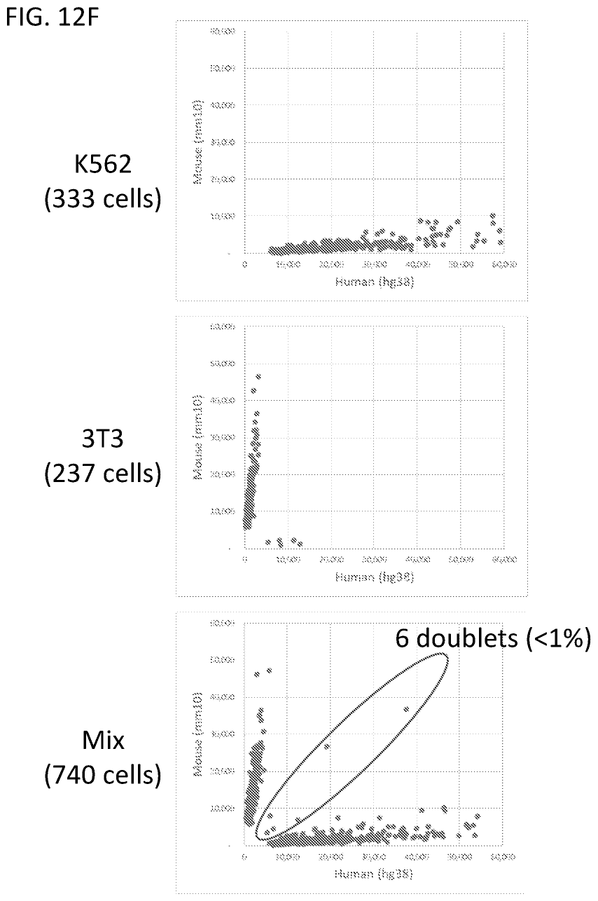

A sequencing library was generated using the protocol illustrated in FIG. 12A. K562 and 3T3 cells were fixed using 1% paraformaldehyde (PFA) and permeabilized with 0.01% digitonin (FIG. 12A, Step B). After washing, the cells were aliquoted to 39 wells of a 96 well plate (8 wells for K562 cells, 8 wells for 3T3 cells and 23 wells for a mixture of K562 and 3T3 cells) such that each well contained approximately 1,000 cells. Reverse transcription mix comprising reverse transcriptase; RNase inhibitor; RT oligo comprising a poly-dT sequence and a PCR handle sequence; template switch oligo (TSO) comprising a first well-specific barcode (BC1) and a primer binding site for PCR; dNTPs and RT buffer, was added to each of the wells, and the RT reaction was performed at 42° C. for 90 minutes (FIG. 12A, Step C). The cells were then collected from each of the wells and pooled together. After centrifugation, the supernatant was discarded, and the cells were resuspended in PBS buffer.

The resuspended cells were redistributed to 1,296 wells of an ICELL8 nanowell chip using an ICELL8 instrument (Takara Bio USA, Inc., San Jose CA) (FIG. 12A, Step E). Two forward PCR primers each comprising one of a pair of partial second round well-specific barcodes (either BC2a and BC2b) were sequentially dispensed into the chip together with a reverse PCR primer that hybridizes to the PCR handle sequence from the RT oligo and PCR reagents containing DNA polymerase, dNTPs and PCR buffer. The two forward primers were added such that one defined a specific row of wells on the chip and the other a specific column of wells. Thus, in combination they define a unique well location. The first forward PCR primer comprises a sequence that can hybridize to the PCR handle provided by the TSO, BC2a, and the Illumina RP2 sequence. The second forward PCR primer comprises the Illumina RP2 sequence, BC2b, and the P7 sequence. Thus, as shown in FIG. 12A, Step F, together these primers enable a “step-out” PCR reaction that ultimately generates a PCR product comprising sequence derived from both primers in combination.

After completion of this second barcoding step, the barcoded full-length cDNAs were extracted from the ICELL8 chip by centrifugation and purified with magnetic beads. After quantification by Qubit and Bioanalyzer High sensitivity kit, the cDNA was used to prepare a sequencing library for differential gene expression analysis (5′DE) using the Illumina Nextera XT kit, followed by PCR using a P7 primer and an Illumina P5 index primer (FIG. 12B). After purification and quantification, the final 5′DE library was loaded on to an Illumina NextSeq for paired-end sequencing (2×75 PE). The resultant sequencing reads were demultiplexed and analyzed using Cogent AP software (Takara Bio USA, Inc., San Jose CA).

After the sequencing reads were demultiplexed using BC1, BC2a, BC2b (i7) and i5 index using the Cogent AP software, 1310 cells having >10,000 reads/cell were identified (FIG. 12C). The data from these cells were used for downstream analysis (as shown in FIGS. 12D-12F). In particular, the L-plot analysis shown in FIG. 12F clearly showed the high mapping rate of the data to either the human genome (hg38; representative of K562 cells) or the mouse genome (mm10; representative of 3T3 cells) with a very low doublet rate. This demonstrated that this combinatorial barcoding strategy can generate single cell data.

### Example 7. Two-Round Barcoding for Combined Targeted Sequencing of TCR Chains and 5′ Differential Expression (5′DE) Using PBMC RNA

PBMC RNA (Takara Bio USA Inc., San Jose CA) was diluted to 5.0 ng/ul, and 2 ul (10 ng) was distributed into an eppendorf tube. Reverse transcription mix comprising reverse transcriptase; RNase inhibitor; RT oligo comprising a poly-dT sequence and a PCR handle sequence; template switch oligo (TSO) comprising a first well-specific barcode (BC1) and a primer binding site for PCR; dNTPs and RT buffer, was added to the tube, and the RT reaction was performed in 20 ul at 42° C. for 90 minutes, followed by incubation at 70° C. for 10 minutes (FIG. 12A, Step C). The RT product with BC1 was purified by magnetic beads and eluted into 12 ul elution buffer.

Two forward PCR primers each comprising one of a pair of partial second round well-specific barcodes (either BC2a or BC2b) were added to the tube together with a reverse PCR primer that hybridizes to the PCR handle sequence from the RT oligo and PCR reagents comprising DNA polymerase, dNTPs and PCR buffer. The two forward primers were added such that one provided a partial second round barcode sequence BC2a and the other provided a partial second round barcode sequence BC2b. Thus, in combination they define a unique second round barcode. The first forward PCR primer comprises a sequence that can hybridize to the PCR handle provided by the TSO, BC2a, and the Illumina RP2 sequence. The second forward PCR primer comprises the Illumina RP2 sequence, BC2b, and the P7 sequence. Thus, as shown in FIG. 12A, Step F, together these primers enable a “step-out” PCR reaction that ultimately generates a PCR product comprising sequence derived from both primers in combination.

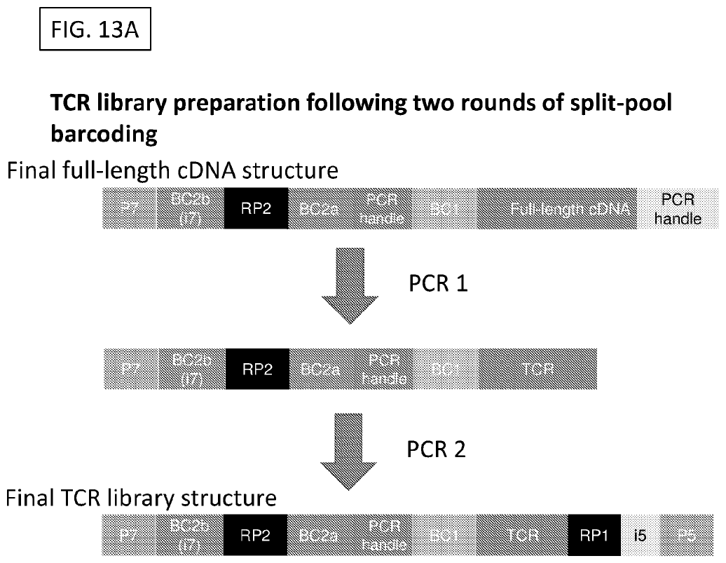

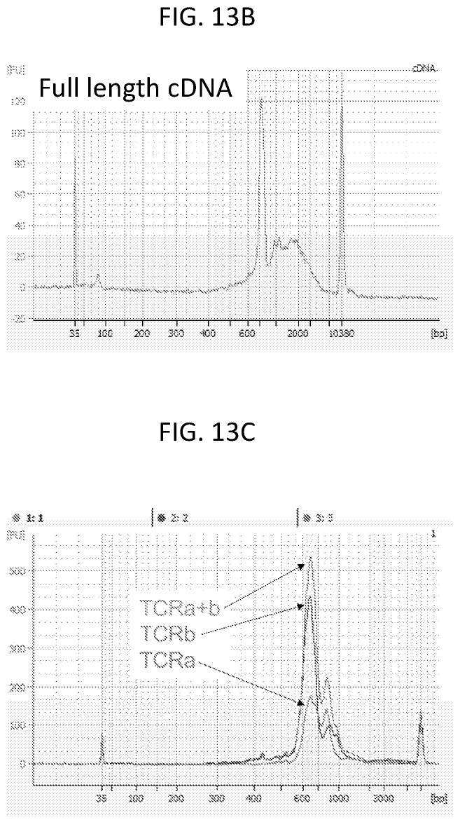

After completion of this second barcoding step with PCR, the barcoded full-length cDNA was purified with magnetic beads. After quantification by Qubit and Bioanalyzer High sensitivity kit (FIG. 13B), the cDNA was aliquoted into 3 tubes as follows:
Tube1 for TCRa,
Tube2 for TCRb, and
Tube3 for TCRa&b.

PCR1 mix comprising DNA polymerase; P7 primer; dNTPs and PCR buffer were added to each of the three tubes. Then, TCRa PCR1 primer, which specifically hybridizes to the constant region of the T-cell antigen receptor (TCR) alpha chain, was added to tube 1. TCRb PCR1 primer, which specifically hybridizes the constant region of the T-cell antigen receptor (TCR) beta chain, was added to tube 2, and both TCRa and TCRb PCR1 primers were added to tube 3. PCR1 was then performed in 40 ul (FIG. 13A). The TCRa PCR1 primer and TCRb PCR1 primer above are both chimeric DNA/RNA oligonucleotides that can work as PCR primers in the absence of RNase, but which can be inactivated by digestion with various RNases (e.g., RNaseH, RNaseA, etc.) after use, e.g., as described in U.S. patent application Ser. No. 16/603,788 published as US 2020-0332341 A1 (Attorney Docket No. CLON-169), the disclosure of which is herein incorporated by reference.

Following PCR1, PCR2 mix comprising DNA polymerase; RNaseH; P5 index primer with i5 index comprising the Illumina RP1 sequence, i5 index, and the P5 adapter sequence; dNTPs; and PCR buffer was added to each of the three tubes along with either TCRa PCR2 primer, which specifically hybridizes the constant region of the T-cell antigen receptor (TCR) alpha chain (tube 1); TCRb PCR2 primer, which specifically hybridizes the constant region of the T-cell antigen receptor (TCR) beta chain (tube 2); or both TCRa and TCRb PCR2 primers (tube 3). Each of the TCRa and b PCR2 primers also comprises an RP1 sequence. PCR2 was then performed in 70 ul (FIG. 13A).

After PCR2, the TCR libraries having all three barcode sequences BC1, BC2a, BC2b (i7) and TCR-specific index (i5) were purified by magnetic beads and quantified by Qubit, Bioanalyzer High sensitivity kit (FIG. 13C) and qPCR. The libraries were loaded on to an Illumina MiniSeq sequencer for paired-end sequencing (2×151 PE), and the resulting sequencing data analyzed with Cogent AP software (Takara Bio USA, Inc., San Jose CA). A summary of the sequencing results in provided in Table 1 below:

TABLE 1

| | Total | Mapping | TCRa | TCRb |
|---|---|---|---|---|
| Sample | Reads | rate | clonotypes | clonotypes |
| Tube 1 | 1,256,775 | 74.0% | 1,207 | 3 |
| Tube 2 | 1,610,347 | 71.5% | 1 | 2,071 |
| Tube 3 | 1,416,166 | 73.0% | 556 | 1,698 |

This result demonstrates that this combinatorial barcoding strategy works.

### Example 8. Three-Round Barcoding for Combined Targeted Sequencing and 5′ Differential Expression (5′DE)

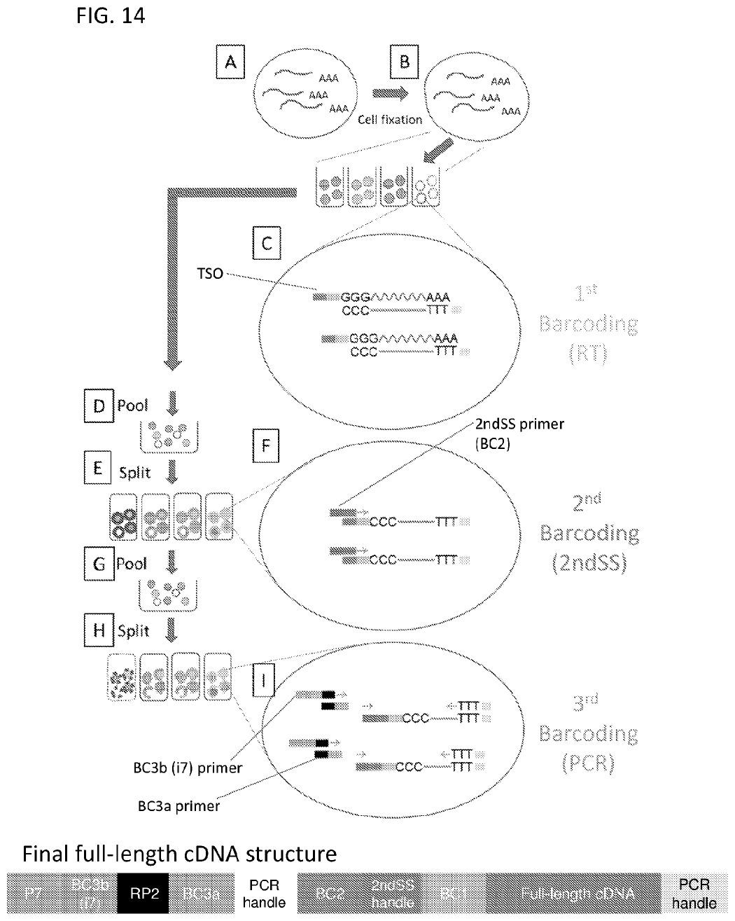

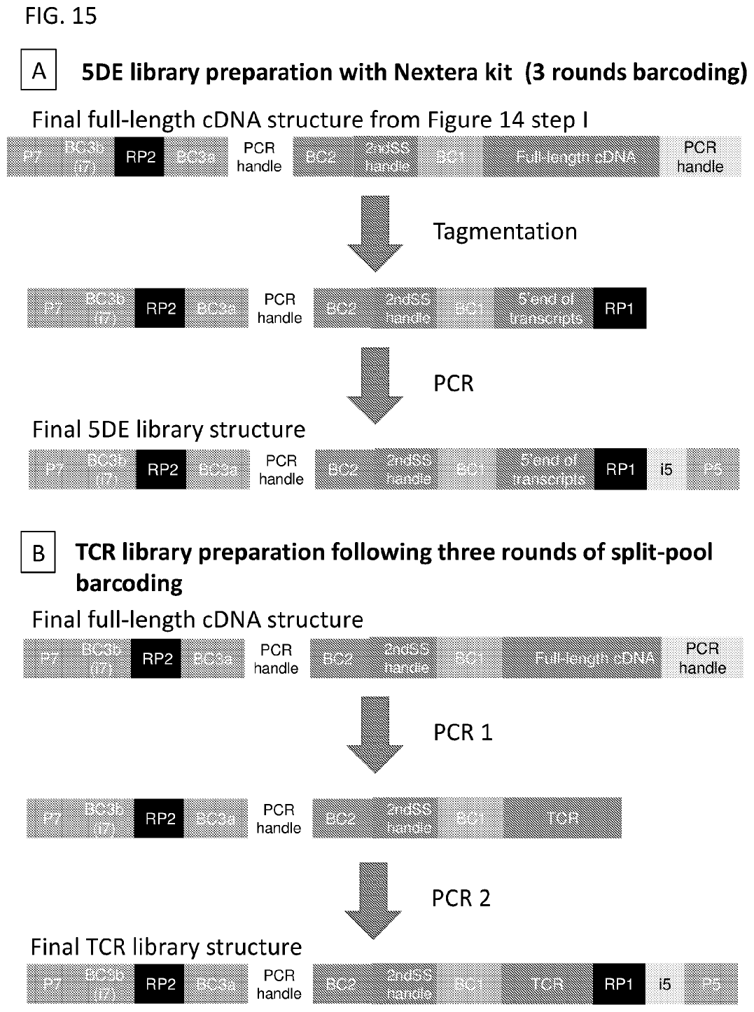

A protocol for generating targeted sequencing and 5′ Differential expression libraries using 3 rounds of barcoding is shown in FIG. 14. In this Example cells are fixed and distributed across wells of a plate (e.g., a 96 well plate). Reverse transcription mix comprising reverse transcriptase, RNase inhibitor, poly-dT oligo, template switch oligo (TSO) comprising a first barcode (BC1) and a primer binding site for second strand synthesis (2ndSS handle), dNTPs and RT buffer, is added to each well, and the RT reaction is performed (FIG. 14, Step C). The TSO used in this reaction is a chimeric DNA/RNA oligonucleotide that works as template switching oligo in the absence of RNase, but can be inactivated after use by digestion with various RNases (e.g., RNaseH, RNaseA, etc.), e.g., as described in U.S. patent application Ser. No. 16/603,788 published as US 2020-0332341 A1 (Attorney Docket No. CLON-169), the disclosure of which is herein incorporated by reference. The cells from each well with BC1 are then pooled (FIG. 14 step D). The pooled cells are then redistributed to a second set of wells in a fresh multi-well plate or nanowell chip (FIG. 14 Step E).

2nd strand synthesis mix (2ndSS) comprising reverse transcriptase; RNaseH; 2ndSS oligo comprising a sequence that hybridizes to the TSO primer binding sequence, BC2 and a PCR handle sequence; dNTPs and 2ndSS reaction buffer, is added to each well. The 2ndSS reaction is then performed (FIG. 14, Step F). The cells comprising 2ndSS product with BC1 and BC2 are then pooled (FIG. 14 step G). The pooled cells are then redistributed to a third set of wells in a fresh multi-well plate or nanowell chip (FIG. 14 Step H).

Two forward PCR primers each comprising one of a pair of partial third round well-specific barcodes (either BC3a or BC3b) are then added to each new well together with a reverse PCR primer that hybridizes to the PCR handle sequence from the RT oligo and PCR reagents containing DNA polymerase, dNTPs and PCR buffer. The two forward primers are added such that one provides a partial third round barcode sequence BC3a and the other provides a partial third round barcode sequence BC3b. Thus, in combination they define a unique third round barcode. The first forward PCR primer comprises a sequence that can hybridize to the PCR handle provided by the second strand synthesis primer from the second round of barcoding, BC3a, and the Illumina RP2 sequence. The second forward PCR primer comprises the Illumina RP2 sequence, BC3b, and the P7 sequence. Thus, as shown in FIG. 14, Step I, together these primers enable a “step-out” PCR reaction that ultimately generates a PCR product comprising sequence derived from both primers in combination.

After completion of this third barcoding step with PCR, the barcoded full-length cDNA can be converted to final 5′DE and/or TCR or other gene-specific libraries using a process similar to that described in either Example 6 (using Nextera (Illumina, Inc., San Diego CA) for 5′ DE) or Example 7 (two rounds PCR for TCR-specific library generation). The steps for this and the structure of the final libraries generated are shown in FIG. 15. FIG. 15 Panel A shows the steps for generating a 5′DE library using tagmentation (e.g., with Nextera, Illumina, Inc., San Diego CA). In other embodiments, combined fragmentation and ligation of hairpin adapters can be used as described in the SMART-Seq Library Prep Kit (Cat. No. 634764, Takara Bio USA, Inc. San Jose CA). FIG. 15 Panel B shows the steps for generating a TCR library following a third round of barcoding.

### Example 9. Analysis of Template Switching Oligos (TSOs) with Different Indices Using Purified RNA

The performance of TSOs with different unique 8-nt indices was tested in a reverse-transcription (RT) reaction. 10 ng purified K562 RNA was mixed with 4 ul RT buffer (250 mM Tris, 375 mM KCl, 30 mM MgCl₂), 1 ul random hexamer, 1 ul nuclease free water, and fragmented by heating at 85 C for 6 min and then immediately cooled down on ice. RT Mastermix containing 4.5 ul TSO buffer, 0.5 ul RNase Inhibitor, 1 ul reverse transcriptase (200 u/ul), 1 ul TSO with 8-nt first identifier (i.e., first index) (50 uM), and 6 ul RNase free water was added to the fragmented RNA. Each well of the 96-well plate contained a TSO with a unique first identifier. The RT reaction was carried out at 25 C 10 min, 42 C 90 min and then 72 C 5 min. After the RT reaction, PCR Mastermix containing 25 ul 2× CB buffer, 1 ul DNA polymerase, 1 ul 5′ PCR Primer and 1 ul 3′ PCR Primer was added to amplify the RT products with the following PCR program: 94 C 1 min; 10 cycles of 98 C 15 s, 55 C 15 s, 68 C 30 s; 68 C 2 min; hold at 4 C. PCR products were purified by magnetic beads and the concentration of PCR product was measured by Qubit.

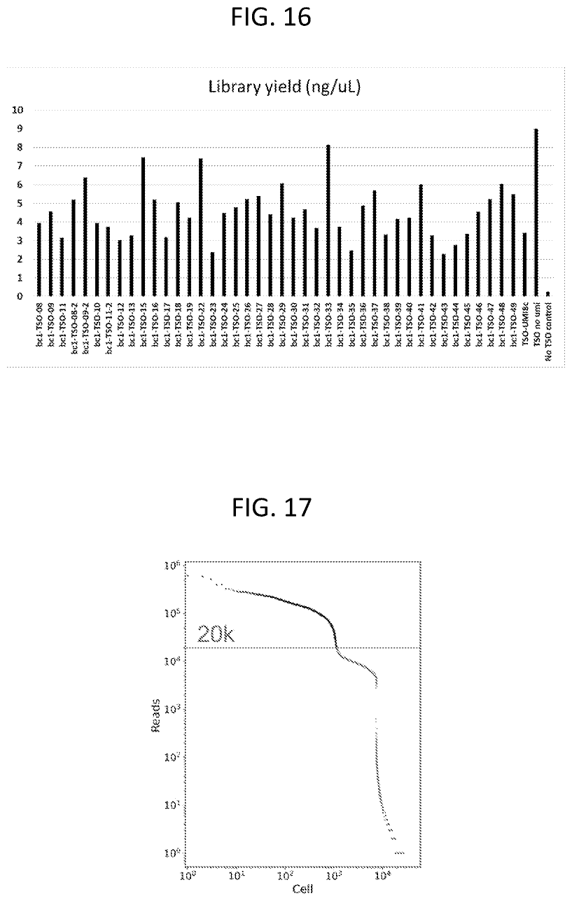

As shown in FIG. 16, all tested TSOs with an 8-nt first identifier generated good library yield. Without TSO, the RT reaction had an extremely low yield. This result demonstrates that adding the first identifier with TSO during the RT step was successful using purified RNA.

The first identifier length can vary from 6-12 nucleotides (nt). Mg²⁺ concentration in the RT buffer can vary from 2-12 mM. The random primer length can vary from 6-15 nt. TSO final concentration in the RT reaction can vary from 0.5-5 uM. Random primer final concentration in the RT reaction can vary from 0.5-5 uM. RNA fragmentation temperature can vary from 65 C-95 C and the incubation time can vary from 1-30 min. RT reaction can be at a constant temperature incubation and can also be temperature gradient with or without thermocycling.

### Example 10. Analysis of Combinatorial Indexing at the Single Cell

Cells or nuclei were fixed in an appropriate fixation solution (e.g., 1-4% paraformaldehyde, Glyoxal, DSP, DST, methanol, etc.). The fixed cells or nuclei were aliquoted to the wells of a multi-well device (e.g., a 96- or 384-well plate, a nano well ICELL8 chip, etc.). Cellular RNAs were fragmented, and reverse transcription Mastermix containing cell permeabilization reagent (e.g., 0.01-0.5% digitonin, saponin, Tween20, Triton X-100, NP40, etc.) was then added to the heat-treated cells. The first identifier was added during reverse-transcription (RT) reaction in situ by use of template switching oligos (TSOs), each carrying a unique first identifier. Then the cells, now containing cDNA with the first identifiers, were pooled and then split again into multiple partitions (e.g., a 96- or 384-well plate, or a 5184 nano well ICELL8 chip (Takara Bio USA, Inc. San Jose CA)), such that each second partition contained multiple cells carrying a different first identifier. PCR Mastermix with primers carrying unique second identifiers was then added into each partition, and PCR reaction was carried out to incorporate the second identifiers into the final library DNA. If desired, cells can go through extra rounds of pooling-splitting and more identifiers could be added by amplification or ligation. The multiple rounds of adding identifier steps were done either manually or by automation, such as with a robotic liquid handler. The final library DNAs from each individual cell have unique combinations of identifiers. rRNA was then depleted from the library, and, after cleaning up and quantification, the library was sequenced on the sequencer (e.g., Miseq, NextSeq, Novaseq, etc., all manufactured by Illumina Inc., San Diego CA)

In this example, cells (K562 (human):3T3 (mouse) at 1:1 ratio) were fixed by 1% paraformaldehyde and aliquoted across the wells of a 96-well plate such that each well contained about 2000 cells per well. RNA fragmentation was carried out by mixing 5 ul of fixed cells with 4 ul RT buffer (250 mM Tris, 375 mM KCl, 30 mM MgCl₂) and 1 ul 12 uM random hexamer, and then heating the cells at 85 C for 6 min. The cells were then immediately cooled down on ice. An RT mix containing 4.5 ul TSO buffer, 1 ul Digitonin (0.2%), 0.5 ul RNase Inhibitor, 1 ul reverse transcriptase (200 u/ul), and 1 ul RNase free water was prepared and added to each well of the plate. 1 ul TSO (50 uM) with a unique first identifier was then added to each well of the 96-well plate, and the RT reaction carried out by incubating the pate at 25 C for 10 min then 42 C for 90 min. After the RT reaction was complete, cells from all wells of the 96-well plate were pooled together and washed once with PBS, leaving 30 ul liquid to resuspend the cells, which were then dispensed across the nano wells of the ICELL8 chip using the ICELL8 instrument (Takara Bio USA, Inc (San Jose CA). Then the i5, i7 indices, used as second identifiers in this experiment, and PCR mix (SeqAmp DNA polymerase and 2× CB buffer, both supplied by Takara Bio USA, Inc. San Jose CA) were dispensed into the ICELL8 chip to mix with the cells. PCR was performed on-chip with the following program: 94 C 1 min; 10 cycles of 100 C 15 s, 49.3 C 5 s, 54.5 C 10 s, 72.2 C 9 s, 67.9 C 31 s; 67.9 C 2 min, hold at 4 C. After the PCR reaction, the library was pooled and cleaned up by magnetic beads. The beads were eluted with ZapR mix (Takara Bio USA, Inc. San Jose CA) containing 2.2 ul 10× ZapR buffer, 1.5 ul scZapR, 1.5 ul heated probe and 16.8 ul Nuclease free water, and the ZapR reaction was carried out at 37 C for 1 h and 72 C for 10 min to remove rRNA from the library. After ZapR was completed, a second PCR reaction was performed to amplify the library by adding 80 ul PCR mix (2 ul SeqAmp DNA polymerase, 2 ul PCR2 Primers, 50 ul 2× CB buffer and 26 ul nuclease-free water) to each tube containing 20 ul ZapR products under the following program: 94 C 1 min; 5 cycles of 98 C 15 s, 55 C 15 s, 68 C 30 s; hold at 4 C. After the second PCR reaction, the products were purified by magnetic beads and quantified by Qubit, BioAnalyzer and qPCR. Based on the quantification, library DNA was diluted to 4 nM. 5 ul 4 nM library was mixed with 5 ul freshly made 0.2N NaOH and incubated at room temperature for 5 min. Then 5 ul of 200 mM Tris-HCl pH7 was added followed by 985 ul HT buffer (Illumina Inc. San Diego CA). The result was a 20 pM denatured library, which was then diluted to 1.5 pM as follows: Denatured library solution (97 μl) and Prechilled HT1 (1203 μl) and loaded to the Nextseq cartridge (Illumina Inc. San Diego CA) for sequencing.

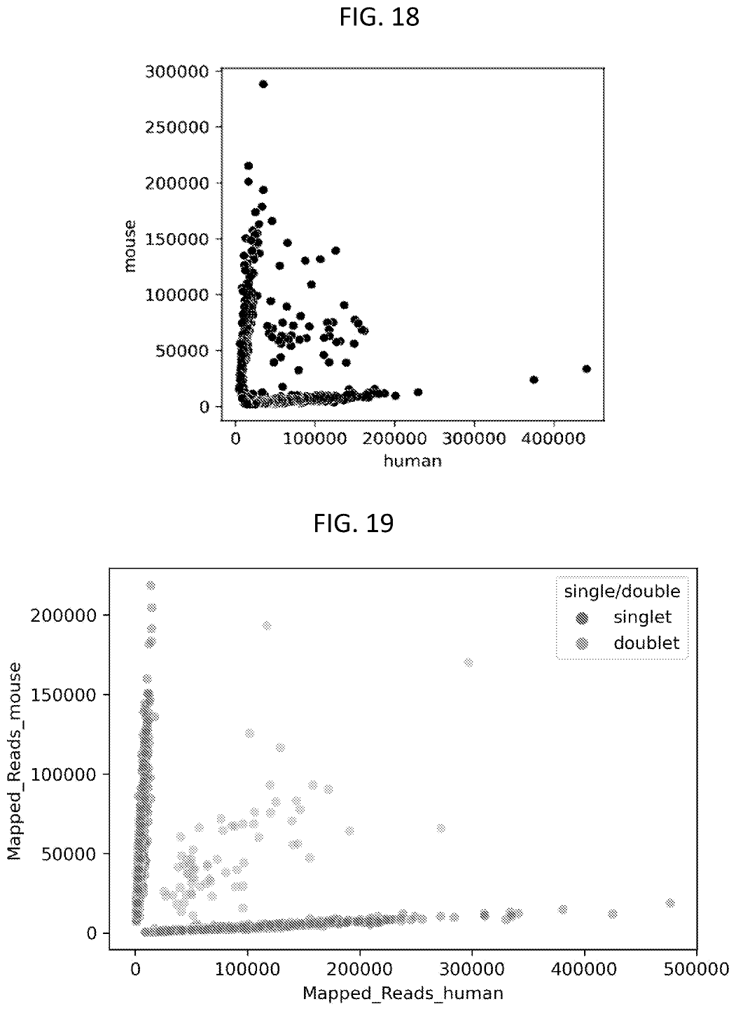

As shown in Table 2, 93.4% of the sequencing reads were successfully barcoded and only 6.6% of the total reads were undetermined. The demultiplexed reads were used for making a “knee plot” to determine the number of cells passing QC that could be identified. As shown in FIG. 17, 1133 cells were successfully barcoded and passed the QC threshold (20,000 reads/cell). This demonstrates that combinatorial indexing was achieved by adding a first identifier using template switching with TSO and by adding a second identifier using PCR. In this case, the second identifier was of the form 2a+2b—ie i5 and i7, which each define either a row or column of the ICELL nano well chip, but combined are unique for a specific well. Combined the incorporated first and second identifiers are unique to each individual single cell from the original pool of fixed cells. The Cogent NGS Analysis Pipeline (Takara Bio USA, Inc. San Jose CA) was used to analyze the sequencing reads and map them to both human and mouse genomes. The mapping results were used to make an “L-Plot”, which was used to determine the percentage of cells captured individually as well as the presence of cell doublets. As shown in FIG. 18, the x-axis of the L-plot shows the reads mapped to the human genome and the y-axis shows reads mapped to the mouse genome. The human and mouse cells were well separated on the L-Plot. The doublet ratio was calculated to be 9.8% which was close to the expected ratio of 10.5%, which was expected based on the number of indices used and the total number of cells assayed. This Demonstrates that there was minimal cell-cell crosstalk during the combinatorial indexing workflow and thus that this method can be used to individually identify single cells. As shown in Table 3, the sequencing data showed good mapping metrics overall with a good Exon and Intron ratio and a low Intergenic/Mitochondrial/Ribosomal ratio. 5896 genes were detected at an average sequencing depth of 148,000 reads/cell. These data demonstrate that the invention as practiced according to this example embodiment is able to analyze single cell total RNA-seq at high-throughput with good performance.

TABLE 2
Demultiplexing results of the SCI-seq experiment

| | Reads count | Ratio |
|---|---|---|
| Barcoded | 168,138,362 | 93.4% |
| Undetermined | 11,807,012 | 6.6% |

TABLE 3
Mapping metrics of the human cells by SCI-seq

| | Mapping metrics |
|---|---|
| Total Exon Reads | 40.0% |
| Total Intron Reads | 38.5% |
| Intergenic Reads | 10.1% |
| Mitochondrial Reads | 2.7% |
| Ribosomal Reads | 5.6% |
| No. of genes | 5896 |
| Seq depth (reads/cell) | 148k |

### Example 11. Analysis of Combinatorial Indexing at the Single Cell Level with High Concentration of PFA and Digitonin

In this example, 4 million cells (K562:3T3 at 1:1 ratio) were centrifuged at 300 g for 3 min. The pellet was resuspended in 1 ml of 4% PFA and incubated on ice for 15 min for cell fixation. Cells were then pelleted by centrifugation at 500 g for 5 min. The cell pellet was resuspended in 1 ml of 3 mM Glycine (pH7.5) and incubated on ice for 5 min to quench the fixation process. Cells were pelleted again by centrifugation at 500 g for 5 min to remove the Glycine solution, and were then resuspended in PBS containing 1% second diluent and 1% RNase inhibitor. 9 ul of the fixed cells was mixed with 4 ul of RT buffer (250 mM Tris, 375 mM KCl, 30 mM MgCl₂) and incubated at 85 C for 6 min for RNA fragmentation. Then, 1 ul of 1.4% digitonin was added to the heated cells, and the mixture incubated at room temperature for 5 min for cell permeabilization. The final digitonin concentration was 0.1%. The RT mix (4.5 ul scTSO mix, 0.5 ul RNase Inhibitor, 1 ul reverse transcriptase (200 u/ul), 1 ul 12 uM random primer) was added to each well of a 96-well plate containing permeabilized cells, and the plate was incubated at 42 C 90 min for the RT reaction. After RT, cells from all wells of the 96-well plate were pooled together and washed once with PBS with 0.04% BSA, leaving 25 ul liquid to resuspend the cells, which were then dispensed into the nano wells of the ICELL8 chip using an ICELL8 instrument (Takara Bio USA, Inc. San Jose CA). Then the i5, i7 indices and PCR mix (SeqAmp DNA polymerase and 2× CB buffer—both from Takara Bio USA, Inc. San Jose CA) were dispensed into the ICELL8 chip to mix with the cells. PCR was performed on-chip with the following program: 72.1 C 3 min; 98.2 C 18 s; 96.5 C 42 s; 10 cycles of 100 C 10 s, 54.4 C 5 s, 59.6 C 10 s, 72.2 C 9 s, 67.9 C 1 min 51 s; hold at 4 C. After the PCR reaction, the library was pooled and cleaned up using magnetic beads. The beads were eluted with ZapR mix (Takara Bio USA, Inc. San Jose CA) containing 2.2 ul 10× ZapR buffer, 1.5 ul scZapR, 1.5 ul heated probe mix and 16.8 ul Nuclease free water. The ZapR reaction (Takara Bio USA Inc. San Jose CA) was then carried out at 37 C for 1 h followed by an incubation at 72 C for 10 min to remove rRNA from the library. After the ZapR reaction was completed, a second PCR reaction was performed to amplify the library by adding 80 ul PCR mix (2 ul SeqAmp DNA polymerase, 2 ul PCR2 Primers, 50 ul 2× CB buffer and 26 ul nuclease-free water) to each tube containing 20 ul ZapR products under the following program: 94 C 1 min; 5 cycles of 98 C 15 s, 55 C 15 s, 68 C 30 s; hold at 4 C. After the PCR reaction was complete, the products were purified by magnetic beads and quantified by Qubit, BioAnalyzer and qPCR. Based on the quantification, library DNA was diluted to 4 nM. 5 ul 4 nM library was mixed with 5 ul freshly made 0.2N NaOH and incubated at room temperature for 5 min. Then 5 ul of 200 mM Tris-HCl pH7 was added followed by 985 ul HT buffer (Illumina Inc. San Diego CA). The result was a 20 pM denatured library, which was then diluted to 1.7 pM and loaded onto a Nextseq cartridge Illumina Inc., San Diego CA) for sequencing.

After sequencing, the Cogent NGS analysis Pipeline (Takara Bio USA, Inc. San Jose CA) was used to analyze the sequencing reads, which were mapped to both the human and mouse genomes. The mapping results were used to make an L-Plot. As shown in FIG. 19, the x-axis showed the reads mapping to the human genome and the y-axis the reads mapping to the mouse genome. The result showed that the human and mouse cells were well separated on the L-Plot. The doublet ratio was 5.8%, which was close to the calculated expected ratio of 5%. This proved that there was minimal cell-cell crosstalk during the combinatorial indexing workflow with 4% PFA and 0.1% digitonin.

### Example 12. Testing Different Cell Fixation Solutions

In this example, K562 cells were used to test different fixation solutions. 2 million cells were washed first with PBS and then split into 5 tubes labeled 1-5. Cells in each tube were pelleted by centrifugation at 200 g for 5 min. In each tube (1-5), the cell pellet was fully resuspended in 0.5 mL of one of either 4% PFA, 1% PFA, 0.5% PFA, 0.25% PFA, or PBS. The cells were then incubated on ice for 10 minutes. 25 ul of 2% digitonin was then added to the cells in each tube for an incubation of 3 min on ice to permeabilize cells. Following permeabilization, 2 mL of quenching solution (1 M Tris-CI pH8, 1% RNase inhibitor, 1% BSA) was added to tubes 1-4 to quench the cell fixation. 2 mL PBS solution (PBS, 1% RNase inhibitor, 1% BSA) was added to tube 5 as a control. The cells were then pelleted by centrifugation at 200 g for 10 minutes at 4 C and resuspended in 200 ul of Resuspension solution (PBS, 1% RNase inhibitor, 1% second diluent, Takara Bio USA Inc. San Jose CA). 10 ul cells in each tube were mixed with 10 ul Trypan blue and checked under a microscope. As shown in Table 4, a wide range of PFA concentrations (0.25%-4%) showed very good cell recovery after fixation and cell permeabilization without forming big cell clumps. The control cells without cell fixation but with digitonin treatment formed obvious big cell clumps with very few single cells. This indicated that a wide range of PFA concentrations can be used for cell fixation for single cell studies.

TABLE 4
Cell fixation condition tests

| | | | | Cell | |
|---|---|---|---|---|---|
| Sample | Cell | Cell | | recovery | Cell cluster |
| ID | fixation | number/ml | | rate | after fixation |
| 1 | 4% PFA | 1.3 | million | 76.5% | Single cells |
| 2 | 1% PFA | 1.4 | million | 82.4% | Single cells |
| 3 | 0.5% PFA | 1.6 | million | 94.1% | Single cells |
| 4 | 0.25% PFA | 1.5 | million | 88.2% | Single cells |

| | | | | |
|---|---|---|---|---|
| 5 | PBS control | 3,000 | 0.2% | Big clumps |

### Example 13. Test of Different Cell Permeabilization Conditions with Titration of Digitonin

In this example, K562 cells were used to test different permeabilization conditions. 2 million cells were washed first with PBS and then pelleted by centrifugation at 200 g for 5 min. The cell pellet was fully resuspended in 0.5 mL of 1% PFA for fixation. Cells were then incubated on ice for 10 minutes. Then 2 mL of quenching solution (1M Tris-CI pH8, 1% RNase inhibitor, 1% BSA) was added to quench the cell fixation. Cells were then pelleted by centrifugation at 200 g for 10 minutes at 4 C and resuspended in 200 ul of Resuspension solution (PBS, 1% RNase inhibitor, 1% second diluent, Takara Bio USA Inc. San Jose CA). Cells were then counted and diluted in Resuspension solution at a concentration of 200, 000 cells/mL. 10 ul cells in each tube were mixed with 10 ul Trypan blue as well as 2 ul of digitonin at two different concentrations and checked under a microscope. As shown in Table 5, cells without digitonin (Sample ID 3) were mostly not stained blue by Trypan blue, indicating that the cell membrane fixed by 1% PFA was not permeable when no digitonin was added. When digitonin (0.1% or 0.01%) was added, cells were all stained blue by Trypan blue (Sample IDs 1 and 2), indicating that digitonin with a wide range of concentrations made the cell membranes permeable.

TABLE 5
Cell permeabilization condition tests

| Sample | | Digitonin | |
|---|---|---|---|
| ID | Fixation | final conc. | Result |
| 1 | 1% PFA | 0.1000% | All Blue |
| 2 | 1% PFA | 0.0100% | All Blue |
| 3 | 1% PFA | NA | Majority not blue |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of preparing a plurality of cell-source identifiable collections of nucleic acids derived from an initial plurality of cellular sources, the method comprising:
(a) providing a first set of cellular source sub-portions, each sub-portion comprising multiple cellular sources of the initial plurality of cellular sources;
(b) generating first identifier tagged nucleic acids in the multiple cellular sources of each sub-portion of the first set using a template switching mediated reaction employing a template switch oligonucleotide comprising a first identifier, wherein the first identifier of the template switch oligonucleotides employed in the different sub-portions of the first set is the same within a given sub-portion but differs between the different sub-portions;
(c) pooling the cellular sources of the sub-portions produced in step b) to produce a first pool of cellular sources comprising first identifier tagged nucleic acids;
(d) apportioning the first pool of cellular sources into a second set of sub-portions each comprising multiple cellular sources comprising first identifier tagged nucleic acids; and
(e) producing cell-source identifiable nucleic acids from the multiple cellular sources in each sub-portion of the second set that include both a first identifier and a second identifier, wherein the second identifiers of each sub-portion of the second set are the same within a given sub-portion but different between different sub-portions;
thereby preparing a plurality of cell-source identifiable collections of nucleic acids from the initial plurality of cellular sources, wherein nucleic acids of each cell-source identifiable collection of nucleic acids comprise a unique combination of first and second identifiers that identifies the cellular source of the nucleic acids.

2. The method according to claim 1, wherein the cellular sources are cells.

3. (canceled)

4. The method according to claim 1, wherein the cellular sources are permeabilized.

5. (canceled)

6. The method according to claim 1, wherein the template switch oligonucleotides further comprise a unique molecular identifier.

7-12. (canceled)

13. The method according to claim 1, wherein the second identifier comprises first and second sub-identifiers.

14. The method according to claim 1, wherein the second identifier is incorporated in a second strand synthesis reaction.

15. The method according to claim 1, wherein the cell source identifiable nucleic acids are produced using an amplification mediated reaction.

16. The method according to claim 1, wherein the cell source identifiable nucleic acids are produced using a ligation mediated reaction.

17. The method according to claim 1, wherein the cell source identifiable nucleic acids are produced using a tagmentation mediated reaction.

18. The method according to claim 1, wherein the method further comprises lysing the multiple cellular sources of the second set of sub-portions.

19. The method according to claim 1, wherein the method further comprises at least one additional pooling/apportioning step to produce nucleic acids that incorporate at least one further identifier.

20. The method according to claim 1, wherein the method further comprises sequencing the plurality of cell-source identifiable collections of nucleic acids.

21. The method according to claim 20, wherein the sequencing comprises next generation sequencing.

22. The method according to claim 1, wherein the method further comprises assigning a cellular source to a cell-source identifiable collection of nucleic acids based on at least the first and second identifier of the nucleic acids of the collection.

23. The method according to claim 1, wherein the cellular sources are immune cellular sources.

24-41. (canceled)

42. A method of preparing a plurality of cell-source identifiable collections of nucleic acids derived from an initial plurality of cellular sources, the method comprising:
(a) providing a first set of cellular source sub-portions, each sub-portion comprising multiple cellular sources of the initial plurality of cellular sources;
(b) generating first identifier tagged nucleic acids in the multiple cellular sources of each sub-portion of the first set using a template switching mediated reaction employing a template switch oligonucleotide comprising a first identifier, wherein the first identifier of the template switch oligonucleotides employed in the different sub-portions of the first set is the same within a given sub-portion but differs between the different sub-portions;
(c) pooling the cellular sources of the sub-portions produced in step (b) to produce a first pool of cellular sources comprising first identifier tagged nucleic acids;
(d) apportioning the first pool of cellular sources into a second set of sub-portions each comprising multiple cellular sources comprising the first identifier tagged nucleic acids;
(e) generating second identifier tagged nucleic acids in the multiple cellular sources of each sub-portion of the second set, wherein the second identifier in the different sub-portions of the second set is the same within a given sub-portion but differs between each of the different sub-portions;
(f) pooling the cellular sources of the sub-portions produced in step (e) to produce a second pool of cellular sources comprising first and second identifier tagged nucleic acids;
(g) apportioning the second pool of cellular sources into a third set of sub-portions each comprising multiple cellular sources comprising the first and second identifier tagged nucleic acids;
(h) producing cell-source identifiable nucleic acids from the multiple cellular sources in each sub-portion of the third set of sub-portions that include a first identifier, a second identifier and a third identifier, wherein the third identifiers of each sub-portion of the third set are the same within a given sub-portion but different between different sub-portions;
thereby preparing a plurality of cell-source identifiable collections of nucleic acids from the initial plurality of cellular sources, wherein nucleic acids of each cell-source identifiable collection of nucleic acids comprise a unique combination of first, second and third identifiers that identifies the cellular source of the nucleic acids.

43. The method according to claim 42, wherein step (e) is performed using a second strand synthesis reaction to add the second identifier.

44. The method according to claim 42, wherein steps (g) and (h) are repeated 1 or more times.

45. The method according to claim 1, wherein the first set of sub-portions comprises at least 2 sub-portions.

46. The method according to claim 1, wherein the second set of sub-portions comprises from at least 2 sub-portions.


SEQUENCE LISTING (22 sequences)

SEQ ID NO: 1 - DNA, 20 bp, synthetic construct
  source 1..20: mol_type = other DNA
aatgatacggcgaccaccga

SEQ ID NO: 2 - DNA, 24 bp, synthetic construct
  source 1..24: mol_type = other DNA
caagcagaagacggcatacgagat

SEQ ID NO: 3 - DNA, 33 bp, synthetic construct
  source 1..33: mol_type = other DNA
acactctttccctacacgacgctcttccgatct

SEQ ID NO: 4 - DNA, 34 bp, synthetic construct
  source 1..34: mol_type = other DNA
gtgactggagttcagacgtgtgctcttccgatct

SEQ ID NO: 5 - DNA, 30 bp, synthetic construct
  source 1..30: mol_type = other DNA
ccatctcatccctgcgtgtctccgactcag

SEQ ID NO: 6 - DNA, 23 bp, synthetic construct
  source 1..23: mol_type = other DNA
cctctctatgggcagtcggtgat

SEQ ID NO: 7 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 8 - DNA, 429 bp, Homo sapiens
  source 1..429: mol_type = genomic DNA
ccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 9 - protein, 138 aa, Mus musculus
PYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 10 - protein, 138 aa, Mus musculus
PNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 11 - DNA, 414 bp, Mus musculus
  source 1..414: mol_type = genomic DNA
ccatacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 12 - DNA, 414 bp, Mus musculus
  source 1..414: mol_type = genomic DNA
ccaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagt

SEQ ID NO: 13 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 14 - DNA, 531 bp, Homo sapiens
  source 1..531: mol_type = genomic DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 15 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 16 - DNA, 537 bp, Homo sapiens
  source 1..537: mol_type = genomic DNA
gacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 17 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - DNA, 522 bp, Mus musculus
  source 1..522: mol_type = genomic DNA
gaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 19 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 20 - DNA, 522 bp, Mus musculus
  source 1..522: mol_type = genomic DNA
gaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 21 - protein, 16 aa, synthetic construct
CASSFSTCSANYGYTF

SEQ ID NO: 22 - protein, 12 aa, synthetic construct
CASSLGTDTQYF